(19) United States
(12) Patent Application Publication
(10) Pub. No.: US 2013/0099208 A1
(43) Pub. Date: Apr. 25, 2013

(54) CONDENSED-CYCLIC COMPOUND, METHOD FOR PREPARING THE CONDENSED-CYCLIC COMPOUND AND ORGANIC LIGHT-EMITTING DEVICE INCLUDING THE CONDENSED-CYCLIC COMPOUND

(71) Applicants: Sun-Young Lee, Yongin-City (KR); Yoon-Hyun Kwak, Yongin-City (KR); Bum-Woo Park, Yongin-City (KR); Se-Jin Cho, Yongin-City (KR)

(72) Inventors: Sun-Young Lee, Yongin-City (KR); Yoon-Hyun Kwak, Yongin-City (KR); Bum-Woo Park, Yongin-City (KR); Se-Jin Cho, Yongin-City (KR)

(73) Assignee: SAMSUNG MOBILE DISPLAY CO., LTD., Yongin-City (KR)

(21) Appl. No.: 13/454,678

(22) Filed: Apr. 24, 2012

(30) Foreign Application Priority Data
Oct. 19, 2011 (KR) 10-2011-0107051

(51) Int. Cl. H01L 51/54; C07D 405/10; C07D 405/14; C07D 409/14; C07D 407/10; C07D 307/77; C07D 409/10; C07D 333/50

(52) U.S. Cl. 257/40; 548/440; 546/284.1; 549/41; 544/216; 548/454; 546/281.1; 549/456; 257/E51.024

(57) ABSTRACT

A condensed-cyclic compound represented by Formula 1 below, a method for preparing the condensed-cyclic compound, and an organic light-emitting device including the condensed-cyclic compound.

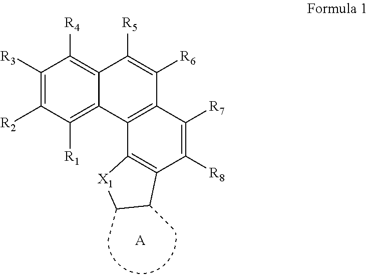

wherein R₁ through R₈, a ring A, and X₁ are defined as in the specification.

### CLAIM OF PRIORITY

This application makes reference to, incorporates the same herein, and claims all benefits accruing under 35 U.S.C. §119 from an application for CONDENSED-CYCLIC COMPOUND, METHOD FOR PREPARING THE CONDENSED-CYCLIC COMPOUND AND ORGANIC LIGHT-EMITTING DEVICE INCLUDING THE CONDENSED-CYCLIC COMPOUND earlier filed in the Korean Intellectual Property Office on 19 Oct. 2011 and there duly assigned Serial No. 10-2011-0107051.

### BACKGROUND OF THE INVENTION

1. Field of the Invention

The present invention relates to a condensed-cyclic compound represented by Formula 1, a method for preparing the condensed-cyclic compound and an organic light-emitting device including the condensed-cyclic compound.

2. Description of the Related Art

Organic light-emitting diodes (OLEDs), which are self-emitting device, have advantages such as a wide viewing angle, excellent contrast, quick response, high brightness, and excellent driving voltage. The OLEDs can provide multicolored images.

A general OLED has a structure including a substrate, an anode, a hole transport layer (HTL), an emission layer (EML), an electron transport layer (ETL), and a cathode which are sequentially stacked on the substrate. In this regard, the HTL, the EML, and the ETL are organic layers formed of organic compounds.

An operating principle of an OLED having the above-described structure is as follows.

When a voltage is applied between the anode and the cathode, holes injected from the anode move to the EML via the HTL, and electrons injected from the cathode move to the EML via the ETL. The holes and electrons recombine in the EML to generate excitons. When the excitons drop from an excited state to a ground state, light is emitted.

### SUMMARY OF THE INVENTION

The present invention provides a condensed-cyclic compound having a novel structure and a method for preparing the same.

The present invention also provides an organic light-emitting device including the condensed-cyclic compound.

According to an aspect of the present invention, there is provided a condensed-cyclic compound represented by Formula 1 below:

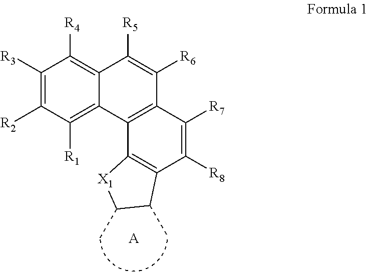

Wherein X₁ may be N(R₁₀), S, or O; a ring A may be a substituted or unsubstituted aromatic ring; R₁ through R₈ and R₁₀ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —Si(R₂₁)(R₂₂)(R₂₃), or —N(R₂₄)(R₂₅); and R₂₁ through R₂₅ may be each independently a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₂-C₆₀ aryloxy group, a substituted or unsubstituted C₅-C₆₀ arylthio group, or a substituted or unsubstituted C₂-C₆₀ heteroaryl group.

According to another aspect of the present invention, there is provided a method of preparing a condensed-cyclic compound represented by Formula 1 above, the method performed according to Reaction Scheme 1 below and comprising: reacting Intermediate c-(1) below with

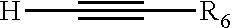

to obtain Intermediate c; reacting the Intermediate c with Intermediate d′ or d″ below to obtain Intermediate e; and cyclizing the Intermediate e to obtain the condensed-cyclic compound of Formula 1:

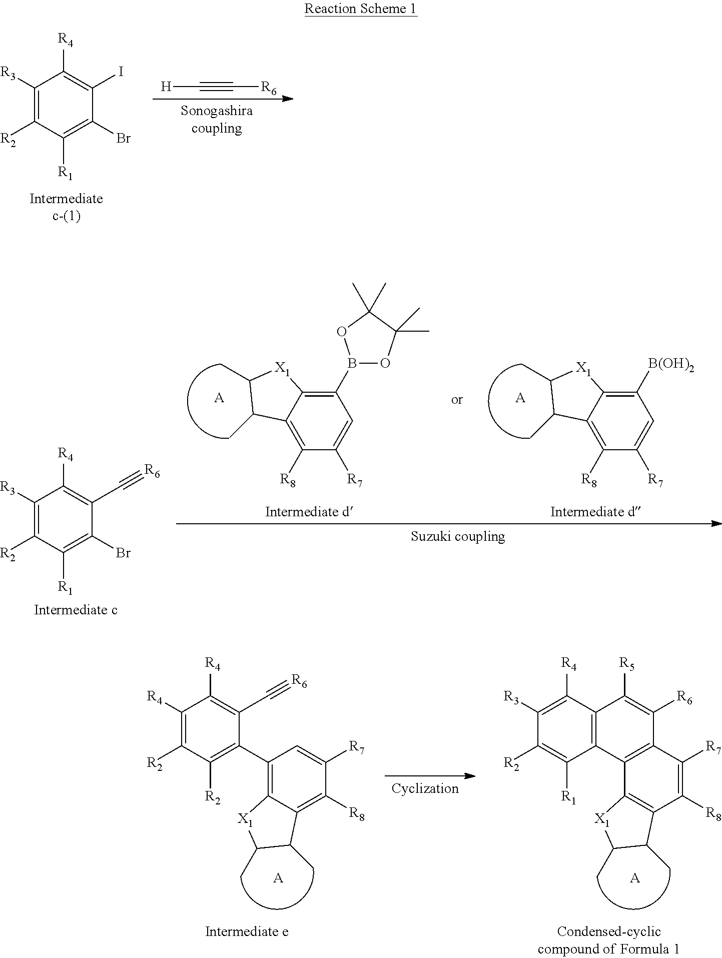

According to another aspect of the present invention, there is provided an organic light-emitting device comprising a first electrode; a second electrode facing the first electrode; and a first layer interposed between the first electrode and the second electrode, wherein the first layer comprises at least one of the condensed-cyclic compounds represented by Formula 1 described above.

### BRIEF DESCRIPTION OF THE DRAWINGS

A more complete appreciation of the present invention, and many of the attendant advantages thereof, will be readily apparent as the present invention becomes better understood by reference to the following detailed description when considered in conjunction with the accompanying drawings, in which like reference symbols indicate the same or similar components, wherein:

FIG. 1 is a schematic diagram illustrating an organic light-emitting diode (OLED) according to an embodiment of the present invention.

### DETAILED DESCRIPTION OF THE INVENTION

Hereinafter, the present invention will be described more fully with reference to the accompanying drawing.

As used herein, the term “and/or” includes any and all combinations of one or more of the associated listed items. Expressions such as “at least one of” when preceding a list of elements, modify the entire list of elements and do not modify the individual elements of the list.

According to an embodiment of the present invention, there is provided a condensed-cyclic compound represented by Formula 1 below:

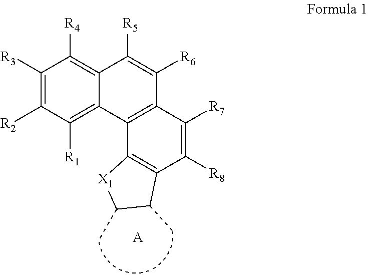

Wherein X₁ may be N(R₁₀), sulfur (S), or oxygen (O). For example, X₁ may be, but is not limited to, S or O.

In Formula 1 above, a ring A may be a substituted or unsubstituted aromatic ring. For example, the ring A may be a substituted or unsubstituted C₅-C₆₀ aromatic ring. The ring A may be a substituted or unsubstituted benzene ring or a substituted or unsubstituted naphthalene ring.

In Formula 1 above, R₁ through R₈ and R₁₀ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted heteroaryl group, —Si(R₂₁)(R₂₂) (R₂₃), or —N(R₂₄)(R₂₅).

For example, R₁₀ may be hydrogen, deuterium, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₁-C₆₀ heteroaryl group.

In this regard, R₂₁ through R₂₅ may be each independently a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

For example, the condensed-cyclic compound of Formula 1 may be represented by Formula 1A, 1B, 1C or 1D:

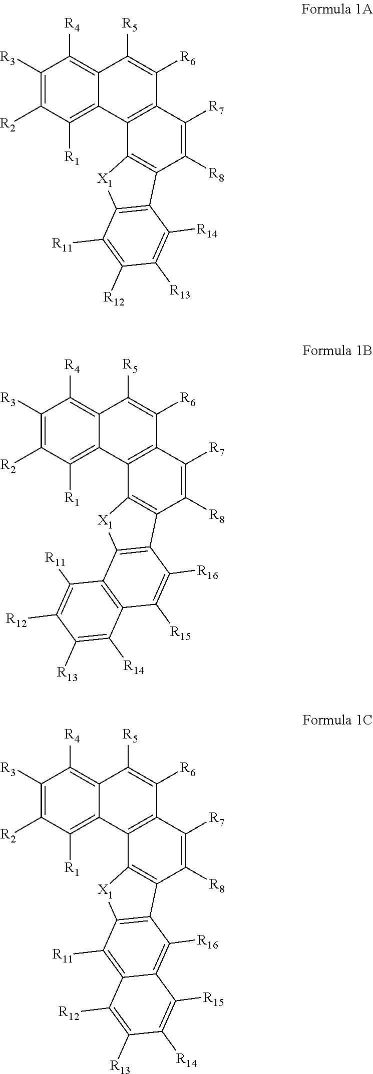

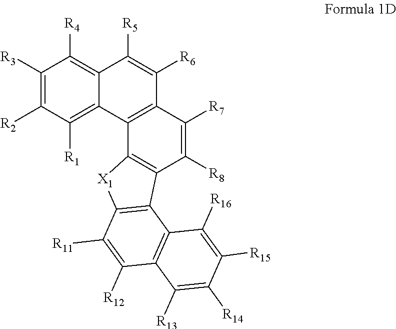

Wherein X₁ and R₁ through R₈ and R₁₀ may be the same as defined herein above and R₁₁ through R₁₆ may be the same as defined herein above with respect to R₁ group.

For example, R₁ through R₈ and R₁₀ through R₁₆ may be each independently, but are not limited to, hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₂₀ alkyl group, a substituted or unsubstituted C₂-C₂₀ alkenyl group, a substituted or unsubstituted C₂-C₆₀ alkynyl group, a substituted or unsubstituted C₁-C₂₀ alkoxy group, —N(R₂₄)(R₂₅), a substituted or unsubstituted phenyl group, a substituted or unsubstituted pentalenyl group, a substituted or unsubstituted indenyl group, a substituted or unsubstituted naphthyl group, a substituted or unsubstituted azulenyl group, a substituted or unsubstituted heptalenyl group, a substituted or unsubstituted indacenyl group, a substituted or unsubstituted acenaphthyl group, a substituted or unsubstituted fluorenyl group, a substituted or unsubstituted phenalenyl group, a substituted or unsubstituted phenanthrenyl group, a substituted or unsubstituted anthryl group, a substituted or unsubstituted fluoranthenyl group, a substituted or unsubstituted triphenylenyl group, a substituted or unsubstituted pyrenyl group, a substituted or unsubstituted chrysenyl group, a substituted or unsubstituted naphthacenyl group, a substituted or unsubstituted picenyl group, a substituted or unsubstituted perylenyl group, a substituted or unsubstituted pentaphenyl group, a substituted or unsubstituted hexacenyl group, a substituted or unsubstituted pyrrolyl group, a substituted or unsubstituted pyrazolyl group, a substituted or unsubstituted imidazolyl group, a substituted or unsubstituted imidazolinyl group, a substituted or unsubstituted imidazopyridinyl group, a substituted or unsubstituted imidazopyrimidinyl group, a substituted or unsubstituted pyridinyl group, a substituted or unsubstituted pyrazinyl group, a substituted or unsubstituted pyrimidinyl group, a substituted or unsubstituted indolyl group, a substituted or unsubstituted purinyl group, a substituted or unsubstituted quinolinyl group, a substituted or unsubstituted phthalazinyl group, a substituted or unsubstituted indolizinyl group, a substituted or unsubstituted naphthyridinyl group, a substituted or unsubstituted quinazolinyl group, a substituted or unsubstituted cinnolinyl group, a substituted or unsubstituted indazolyl group, a substituted or unsubstituted carbazolyl group, a substituted or unsubstituted phenazinyl group, a substituted or unsubstituted phenanthridinyl group, a substituted or unsubstituted pyranyl group, a substituted or unsubstituted chromenyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted isothiazolyl group, a substituted or unsubstituted benzoimidazolyl group, a substituted or unsubstituted isoxazolyl group, a substituted or unsubstituted dibenzothiophenyl group, a substituted or unsubstituted dibenzofuranyl group, a substituted or unsubstituted triazinyl group, or a substituted or unsubstituted oxadiazolyl group. In this regard, R₂₄ and R₂₅ may be each independently a substituted or unsubstituted C₁-C₂₀ alkyl group, a substituted or unsubstituted alkenyl group, a substituted or unsubstituted C₅-C₂₀ aryl group, or a substituted or unsubstituted C₂-C₂₀ heteroaryl group.

For example, R₁ through R₈ and R₁₀ through R₁₆ may be each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₁₀ alkyl group, a substituted or unsubstituted C₁-C₁₀ alkoxy group, —N(R₂₄)(R₂₅) (wherein R₂₄ and R₂₅ may be each independently one selected from a phenyl group; a naphthyl group; an anthryl group; and a phenyl group, a naphthyl group and an anthryl group that is substituted with at least one of deuterium, a cyano group, a halogen atom, CH₂F, CHF₂, and CF₃), and Formulae 2A through 2P below:

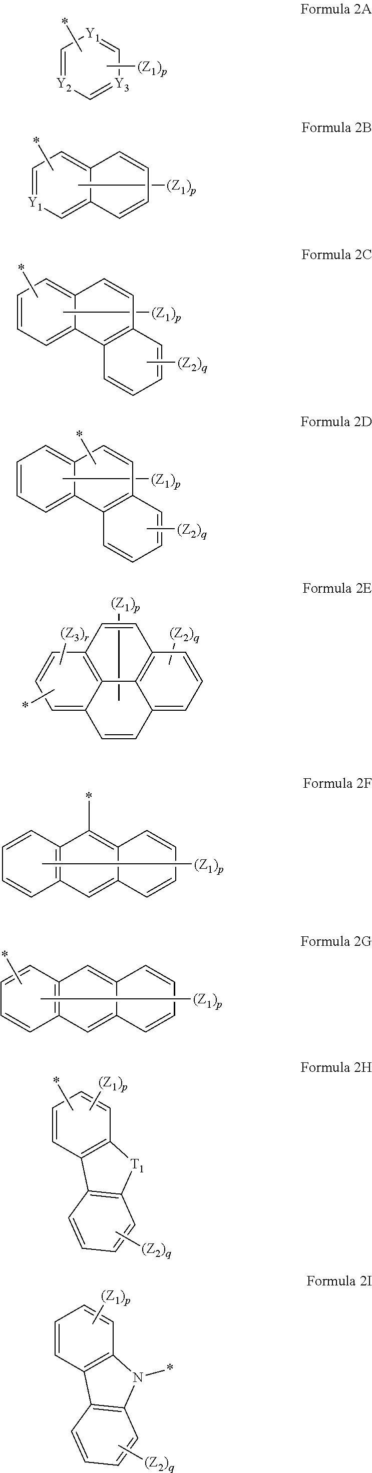

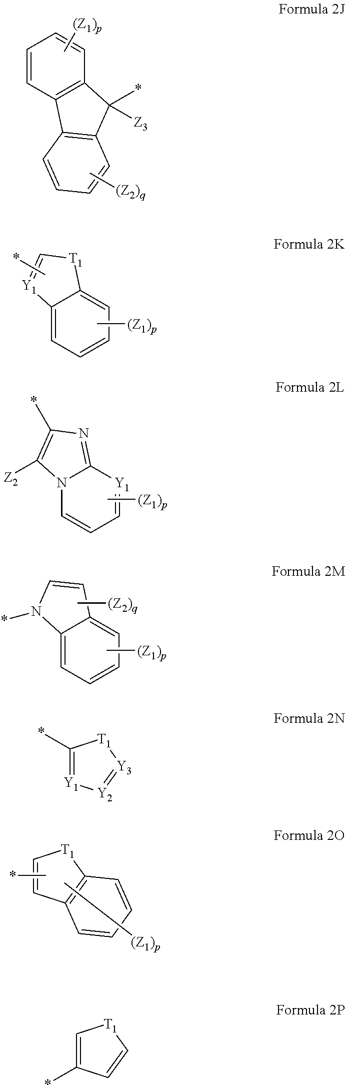

Wherein Y₁ through Y₃ may be each independently ═N— or ═C(Z₁₁)—.

In Formulae 2A through 2P above, T₁ may be —S—, —O—, —N(Z₁₂)—, or —C(Z₁₃)(Z₁₄)—.

In Formulae 2A through 2P above, Z₁ through Z₃ and Z₁₁ through Z₁₄ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₆₀ alkyl group; a C₂-C₆₀ alkenyl group; a C₂-C₆₀ alkynyl group; a C₁-C₆₀ alkoxy group; a C₃-C₆₀ cycloalkyl group; a C₅-C₆₀ aryl group; a C₅-C₆₀ aryloxy group; a C₅-C₆₀ arylthio group; a C₂-C₆₀ heteroaryl group; a C₁-C₆₀ alkyl group, a C₂-C₆₀ alkenyl group, a C₂-C₆₀ alkynyl group, a C₁-C₆₀ alkoxy group, a C₃-C₆₀ cycloalkyl group, a C₅-C₆₀ aryl group, a C₅-C₆₀ aryloxy group, a C₅-C₆₀ arylthio group, and a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group; —N(Q₁)(Q₂); and —Si(Q₃)(Q₄)(Q₅). In this regard, Q₁ through Q₅ may be each independently one of a C₃-C₆₀ cycloalkyl group; a C₅-C₆₀ aryl group; a C₅-C₆₀ aryloxy group; a C₅-C₆₀ arylthio group; a C₂-C₆₀ heteroaryl group; and a C₁-C₆₀ alkyl group, a C₂-C₆₀ alkenyl group, a C₂-C₆₀ alkynyl group, a C₁-C₆₀ alkoxy group, a C₃-C₆₀ cycloalkyl group, a C₅-C₆₀ aryl group, a C₅-C₆₀ aryloxy group, a C₅-C₆₀ arylthio group, and a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₁-C₆₀ alkynyl group, and a C₁-C₆₀ alkoxy group.

For example, Z₁ through Z₃ and Z₁₁ through Z₁₄ may be each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a methyl group; an ethyl group; a propyl group; a butyl group; a pentyl group; a methoxy group; an ethoxy group; a propoxy group; a butoxy group; a pentoxy group; a methyl group, an ethyl group, a propyl group, a butyl group, a pentyl group, a methoxy group, an ethoxy group, a propoxy group, a butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; a benzoimidazolyl group; a phenyl-benzoimidazolyl group; a carbazolyl group, an imidazolyl group, an imidazolinyl group, an imidazopyridinyl group, an imidazopyrimidinyl group, a pyridinyl group, a pyrimidinyl group, and a triazinyl group, a quinolinyl group, a benzoimidazolyl group, and a phenyl-benzoimidazolyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group; and —N(Q₁)(Q₂). In this regard, Q₁ and Q₂ may be each independently one of a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof; a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group.

In Formulae 2A through 2O above, p may be an integer of 1 to 9, q may be an integer of 1 to 4, and r may be an integer of 1 to 3. If p is 2 or more, at least two Z₁ groups may be identical to or different from each other. If q is 2 or more, at least two Z₂ groups may be identical to or different from each other. If r is 2 or more, at least two Z₃ may be identical to or different from each other.

R₁ through R₈ and R₁₀ through R₁₆ may be each independently, but are not limited to, hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group (e.g., methyl, ethyl, propyl, butyl, pentyl, hexyl, and the like), a C₁-C₁₀ alkoxy group (e.g., methoxy, ethoxy, propoxy, butoxy, pentoxy, and the like), a substituted or unsubstituted phenyl group, a substituted or unsubstituted naphthyl group, a substituted or unsubstituted fluorenyl group, a substituted or unsubstituted phenanthrenyl group, a substituted or unsubstituted anthryl group, a substituted or unsubstituted pyrenyl group, a substituted or unsubstituted pyridinyl group, a substituted or unsubstituted indolyl group, a substituted or unsubstituted carbazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted dibenzothiophenyl group, a substituted or unsubstituted dibenzofuranyl group, or a substituted or unsubstituted triazinyl group.

In Formula 1 above, R₁ through R₈ and R₁₀ through R₁₆ may be each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group (e.g., methyl, ethyl, propyl, butyl, pentyl, hexyl, and the like), a C₁-C₁₀ alkoxy group (e.g., methoxy, ethoxy, propoxy, butoxy, pentoxy, and the like), and Formulae 3-1 through 3-40 below:

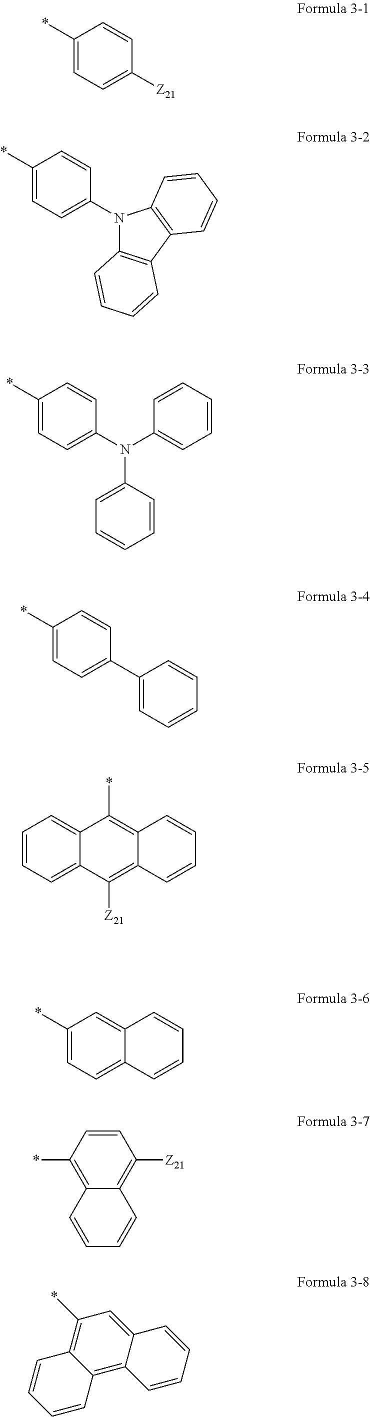

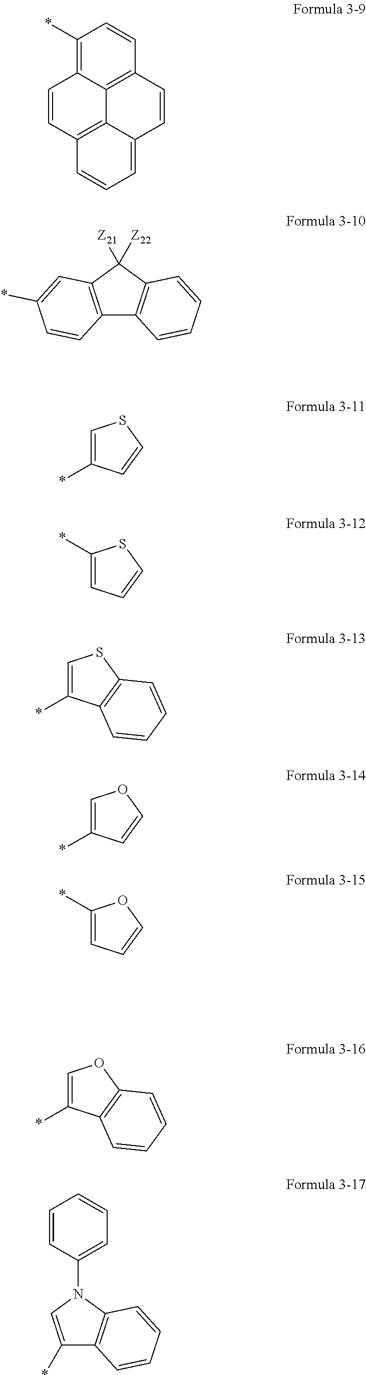

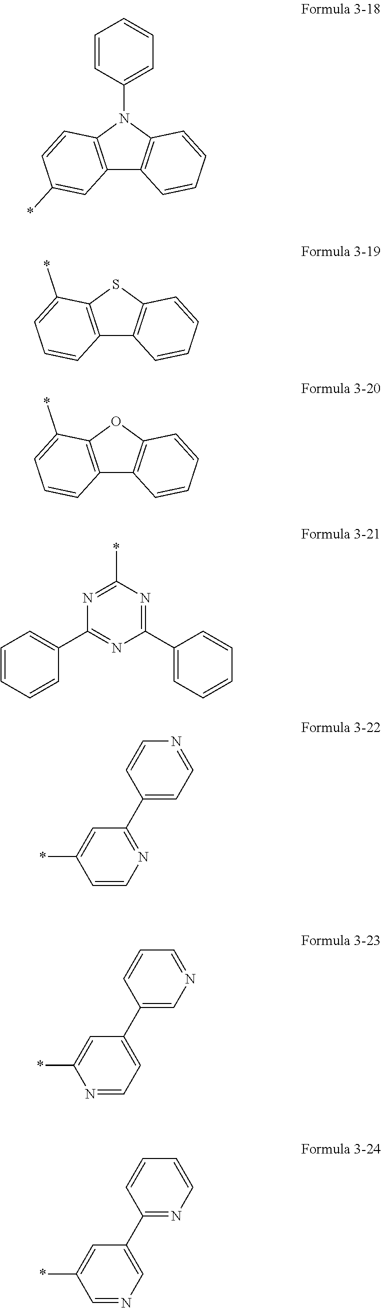

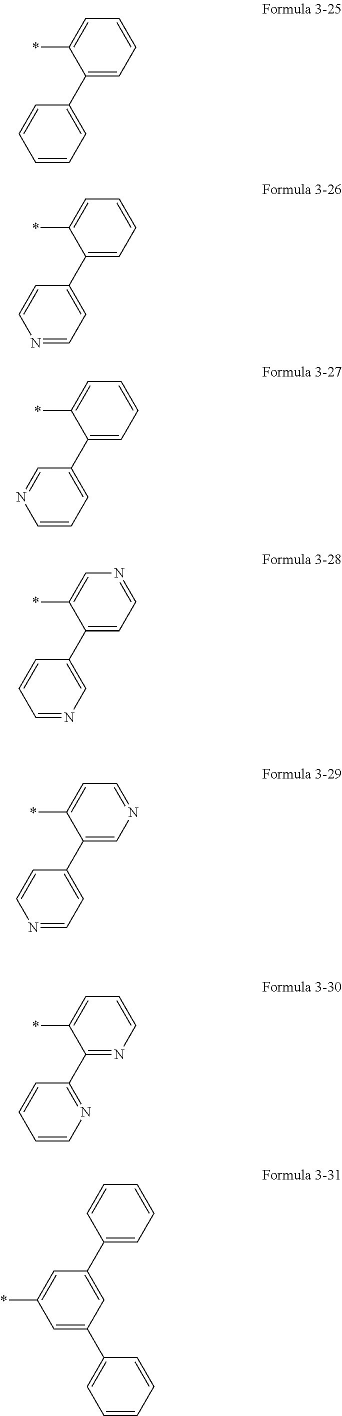

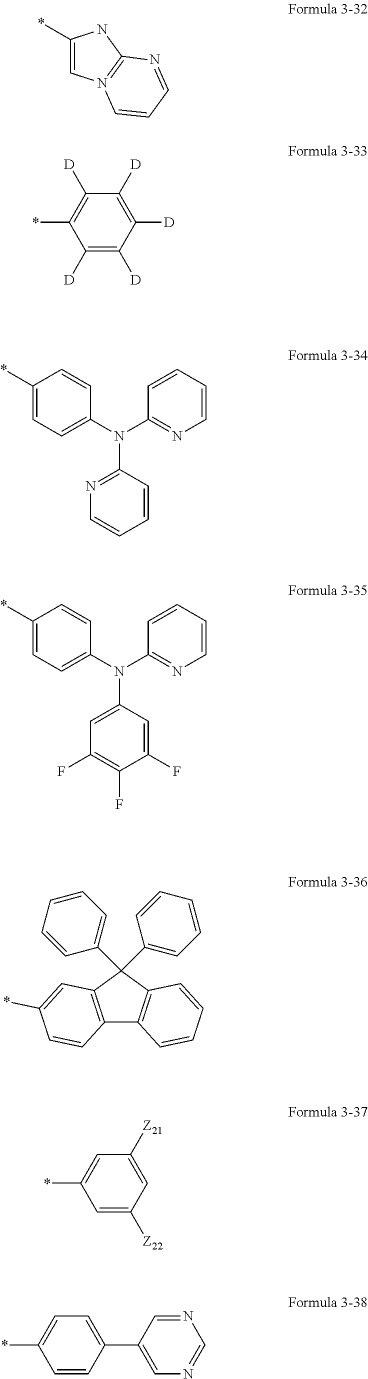

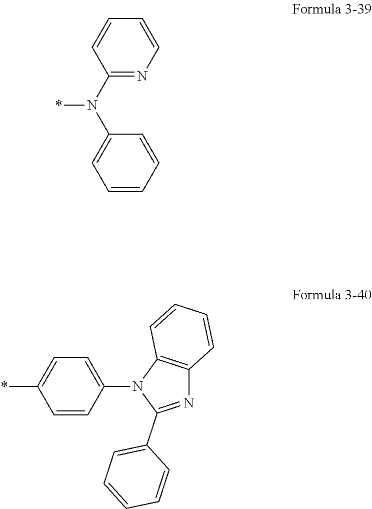 [SMILES: *N(c1ccccc1)c1ccccn1 ; *c1ccc(-n2c(-c3ccccc3)nc3ccccc32)cc1]

In Formulae 3-1 through 3-40 above, Z₂₁ and Z₂₂ may be each independently one selected from hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, a pentoxy group; and a methyl group, an ethyl group, a propyl group, a butyl group, a pentyl group, a methoxy group, an ethoxy group, a propoxy group, a butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof.

In the condensed-cyclic compounds described above, R₇, R₈, and R₁₁ through R₁₆ may be each independently, but are not limited to, hydrogen.

For example, in Formulae 1A through 1D above, R₇, R₈, and R₁₁ through R₁₆ may be each independently hydrogen. In other words, the condensed-cyclic compound of Formula 1 may be represented by one of Formulae 1A-1 through 1D-3, but is not limited thereto:

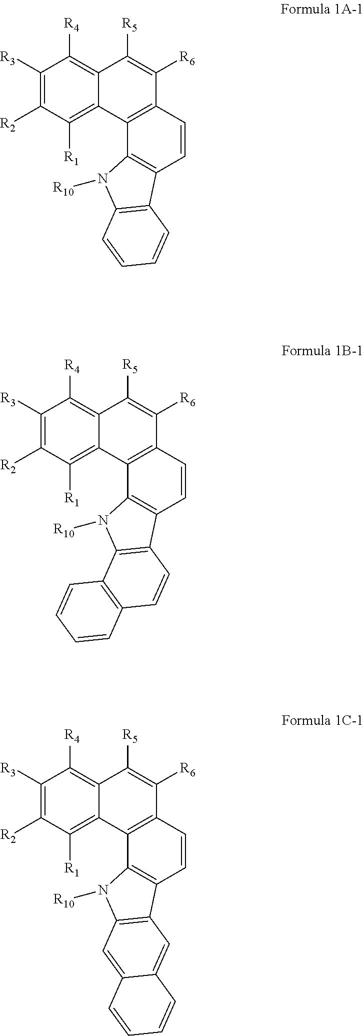

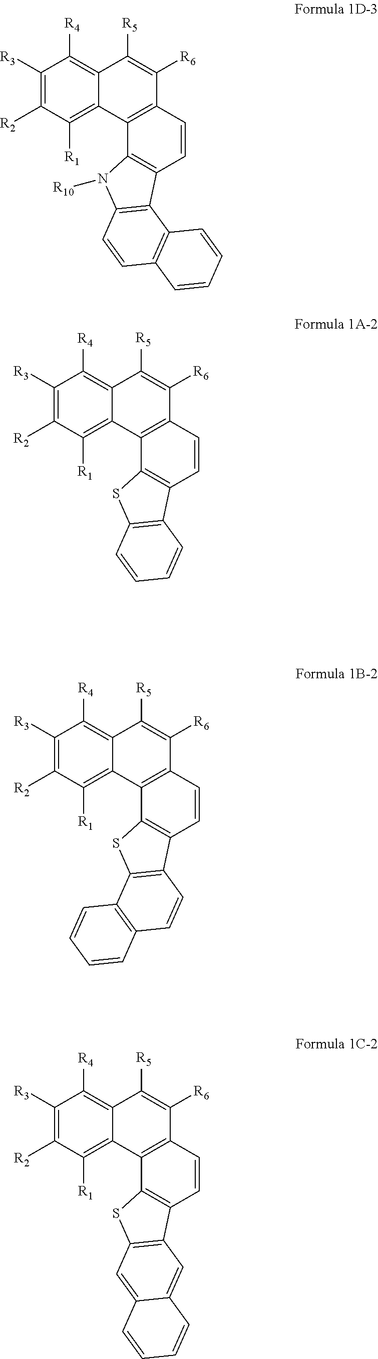

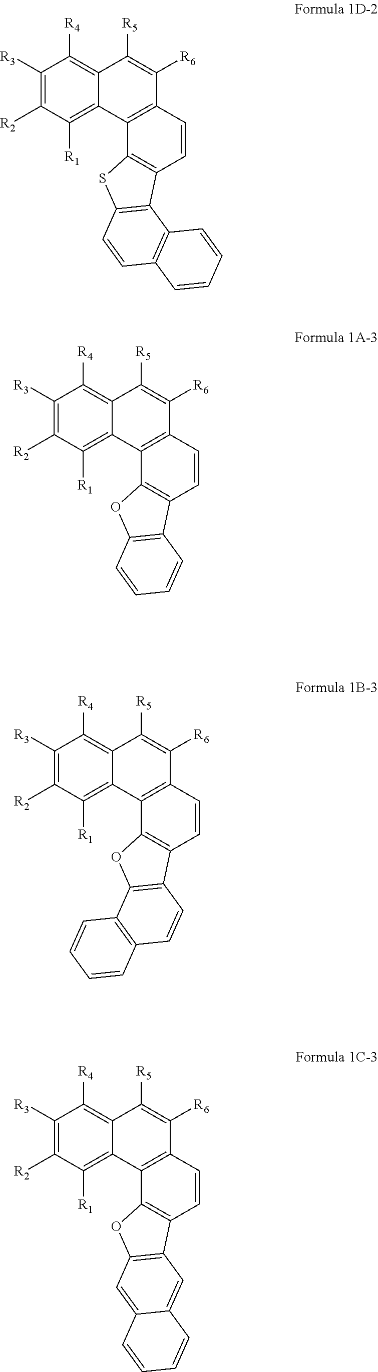

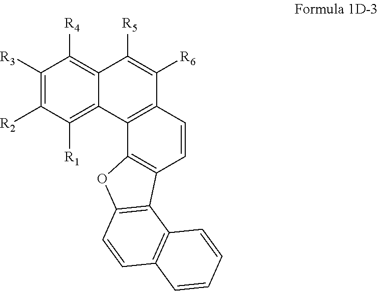

In Formulae 1A-1 through 1D-3 above, R₁ through R₆ and R₁₀ are the same as defined herein.

For example, the condensed-cyclic compound of Formula 1 may be represented by any one of Formulae 1A-2, 1B-2, 1C-2, 1D-2, 1A-3, 1B-3, 1C-3, and 1D-3, but is not limited thereto.

In another embodiment, the condensed-cyclic compound may be represented by Formula 1A below:

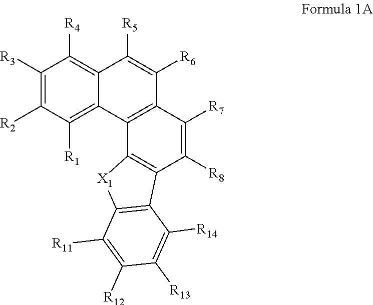

Wherein R₁ through R₈ are each independently hydrogen, deuterium, a halogen atom, a hydroxyl group, a cyano group, a nitro group, an amino group, an amidino group, hydrazine, hydrazone, a carboxyl group or a salt thereof; a sulfonic acid group or a salt thereof, a phosphoric acid or a salt thereof, a substituted or unsubstituted phenyl group, a substituted or unsubstituted naphthyl group, a substituted or unsubstituted fluorenyl group, a substituted or unsubstituted phenanthrenyl group, a substituted or unsubstituted anthryl group, a substituted or unsubstituted pyrenyl group, a substituted or unsubstituted pyridinyl group, a substituted or unsubstituted indolyl group, a substituted or unsubstituted carbazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted dibenzothiophenyl group, a substituted or unsubstituted dibenzofuranyl group, or a substituted or unsubstituted triazinyl group; X₁ is S or O; R₁₁ through R₁₄ may be each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₂₀ alkyl group, a substituted or unsubstituted alkenyl group, a substituted or unsubstituted C₂-C₂₀ alkynyl group, or a substituted or unsubstituted C₁-C₂₀ alkoxy group.

For example, in Formula 1A, R₁ through R₈ may be each independently, but are not limited to, one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof and Formulae 3-1 through 3-40, and R₁₁ through R₁₄ may be each independently, but are not limited to, hydrogen.

The condensed-cyclic compound may be, for example, any one of Compounds 1 through 76 below, but is not limited thereto:

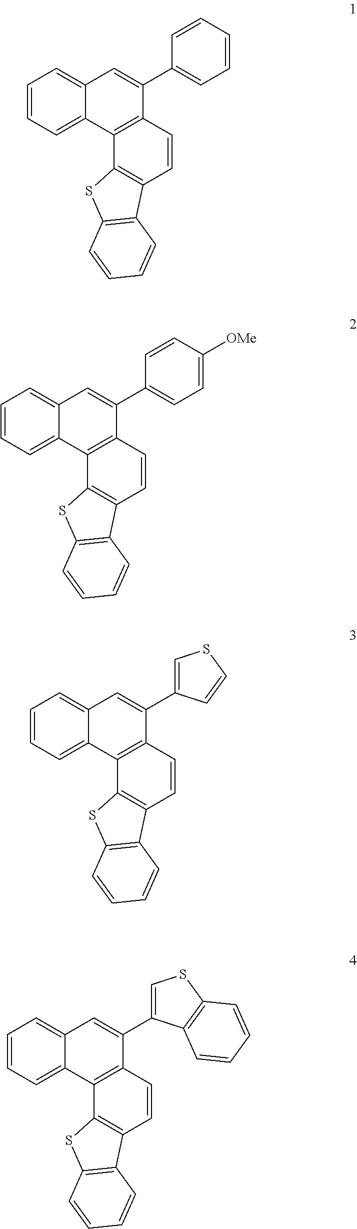 [SMILES: c1ccc(-c2cc3ccccc3c3c2ccc2c4ccccc4sc23)cc1 ; COc1ccc(-c2cc3ccccc3c3c2ccc2c4ccccc4sc23)cc1 ; c1ccc2c(c1)cc(-c1ccsc1)c1ccc3c4ccccc4sc3c12 ; c1ccc2c(c1)cc(-c1csc3ccccc13)c1ccc3c4ccccc4sc3c12]

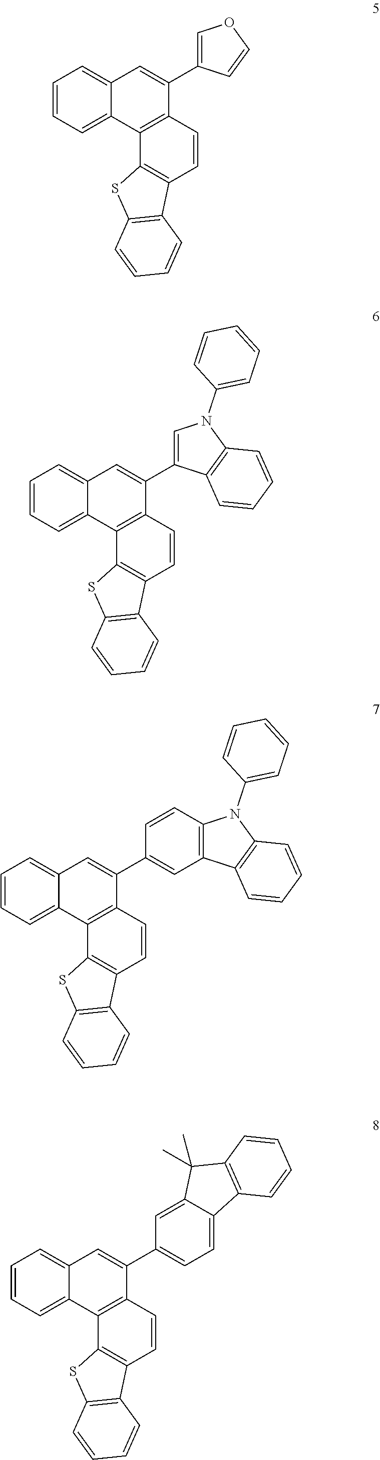 [SMILES: c1ccc2c(c1)cc(-c1ccoc1)c1ccc3c4ccccc4sc3c12 ; c1ccc(-n2cc(-c3cc4ccccc4c4c3ccc3c5ccccc5sc34)c3ccccc32)cc1 ; c1ccc(-n2c3ccccc3c3cc(-c4cc5ccccc5c5c4ccc4c6ccccc6sc45)ccc32)cc1 ; CC1(C)c2ccccc2-c2ccc(-c3cc4ccccc4c4c3ccc3c5ccccc5sc34)cc21]

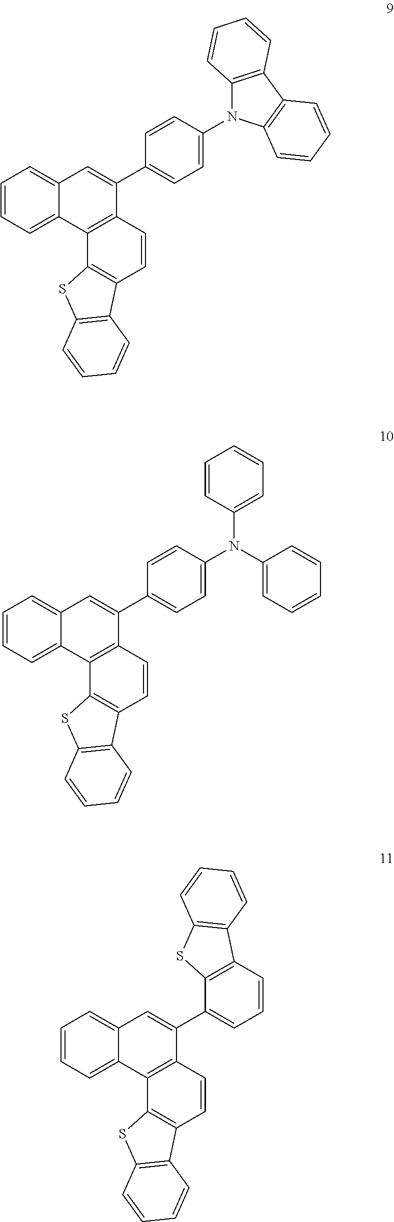 [SMILES: c1ccc2c(c1)cc(-c1ccc(-n3c4ccccc4c4ccccc43)cc1)c1ccc3c4ccccc4sc3c12 ; c1ccc(N(c2ccccc2)c2ccc(-c3cc4ccccc4c4c3ccc3c5ccccc5sc34)cc2)cc1 ; c1ccc2c(c1)cc(-c1cccc3c1sc1ccccc13)c1ccc3c4ccccc4sc3c12]

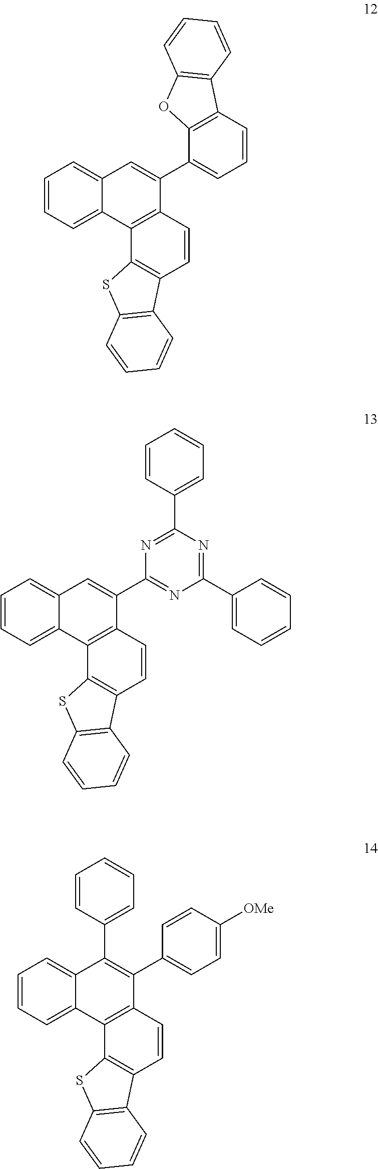 [SMILES: c1ccc2c(c1)cc(-c1cccc3c1oc1ccccc13)c1ccc3c4ccccc4sc3c12 ; c1ccc(-c2nc(-c3ccccc3)nc(-c3cc4ccccc4c4c3ccc3c5ccccc5sc34)n2)cc1 ; COc1ccc(-c2c(-c3ccccc3)c3ccccc3c3c2ccc2c4ccccc4sc23)cc1]

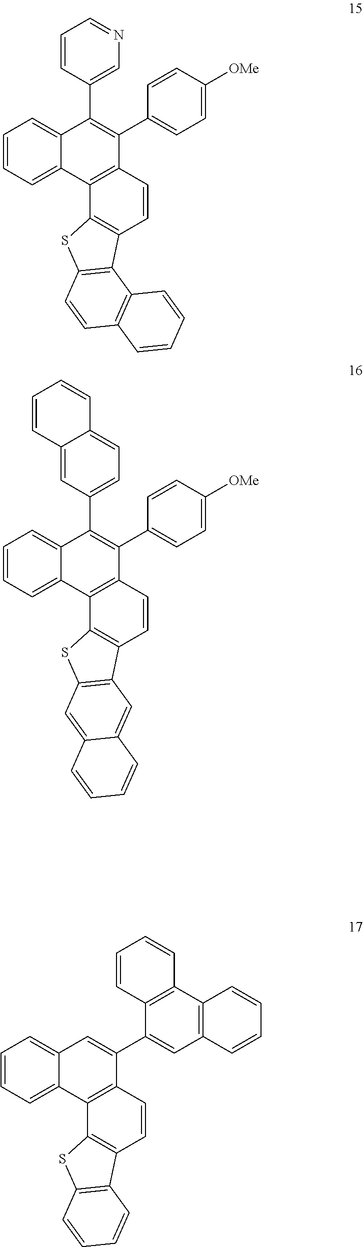 [SMILES: COc1ccc(-c2c(-c3cccnc3)c3ccccc3c3c2ccc2c3sc3ccc4ccccc4c32)cc1 ; COc1ccc(-c2c(-c3ccc4ccccc4c3)c3ccccc3c3c2ccc2c4cc5ccccc5cc4sc23)cc1 ; c1ccc2c(c1)cc(-c1cc3ccccc3c3c1ccc1c4ccccc4sc13)c1ccccc12]

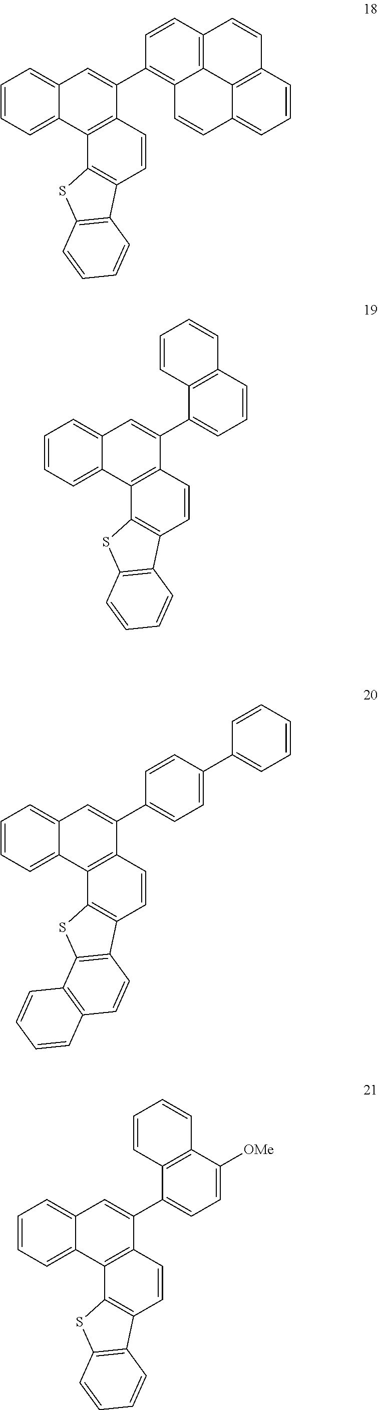 [SMILES: c1ccc2c(c1)cc(-c1ccc3ccc4cccc5ccc1c3c45)c1ccc3c4ccccc4sc3c12 ; c1ccc2c(-c3cc4ccccc4c4c3ccc3c5ccccc5sc34)cccc2c1 ; c1ccc(-c2ccc(-c3cc4ccccc4c4c3ccc3c5ccc6ccccc6c5sc34)cc2)cc1 ; COc1ccc(-c2cc3ccccc3c3c2ccc2c4ccccc4sc23)c2ccccc12]

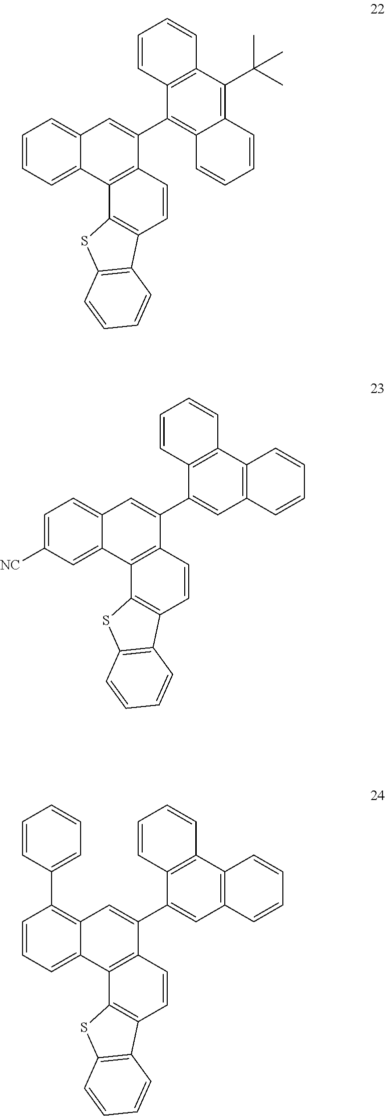 [SMILES: CC(C)(C)c1c2ccccc2c(-c2cc3ccccc3c3c2ccc2c4ccccc4sc23)c2ccccc12 ; N#Cc1ccc2cc(-c3cc4ccccc4c4ccccc34)c3ccc4c5ccccc5sc4c3c2c1 ; c1ccc(-c2cccc3c2cc(-c2cc4ccccc4c4ccccc24)c2ccc4c5ccccc5sc4c23)cc1]

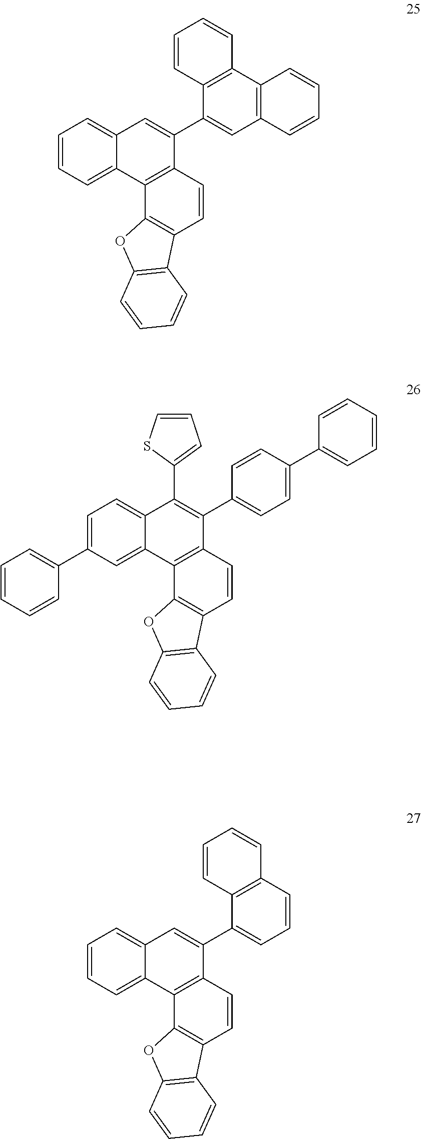 [SMILES: c1ccc2c(c1)cc(-c1cc3ccccc3c3c1ccc1c4ccccc4oc13)c1ccccc12 ; c1ccc(-c2ccc(-c3c(-c4cccs4)c4ccc(-c5ccccc5)cc4c4c3ccc3c5ccccc5oc34)cc2)cc1 ; c1ccc2c(-c3cc4ccccc4c4c3ccc3c5ccccc5oc34)cccc2c1]

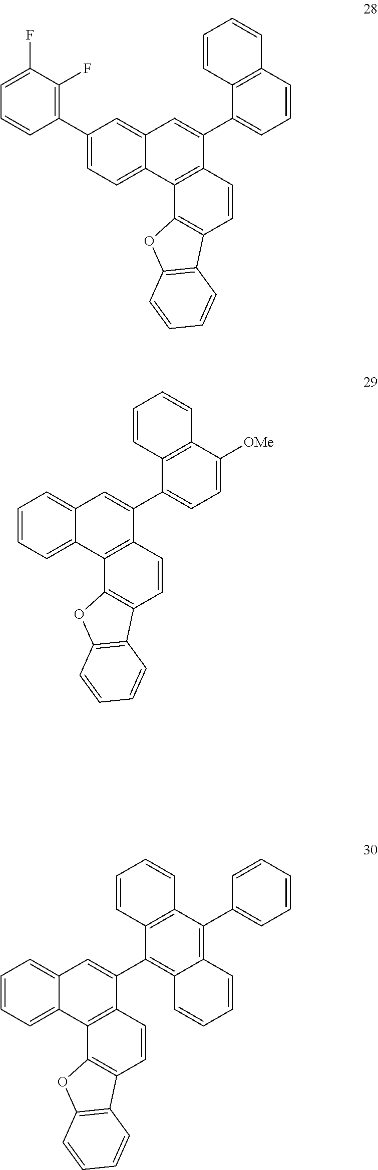 [SMILES: Fc1cccc(-c2ccc3c(c2)cc(-c2cccc4ccccc24)c2ccc4c5ccccc5oc4c23)c1F ; COc1ccc(-c2cc3ccccc3c3c2ccc2c4ccccc4oc23)c2ccccc12 ; c1ccc(-c2c3ccccc3c(-c3cc4ccccc4c4c3ccc3c5ccccc5oc34)c3ccccc23)cc1]

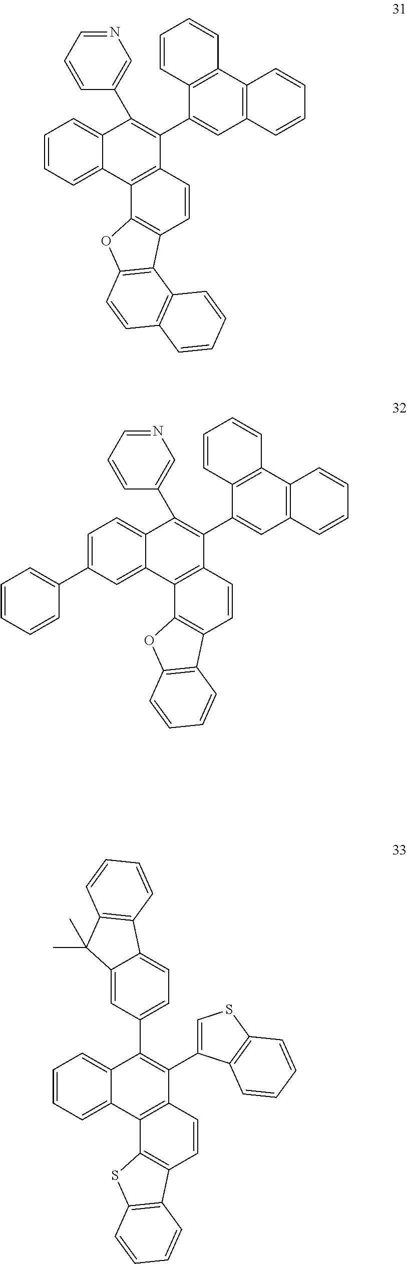 [SMILES: c1cncc(-c2c(-c3cc4ccccc4c4ccccc34)c3ccc4c(oc5ccc6ccccc6c54)c3c3ccccc23)c1 ; c1ccc(-c2ccc3c(-c4cccnc4)c(-c4cc5ccccc5c5ccccc45)c4ccc5c6ccccc6oc5c4c3c2)cc1 ; CC1(C)c2ccccc2-c2ccc(-c3c(-c4csc5ccccc45)c4ccc5c6ccccc6sc5c4c4ccccc34)cc21]

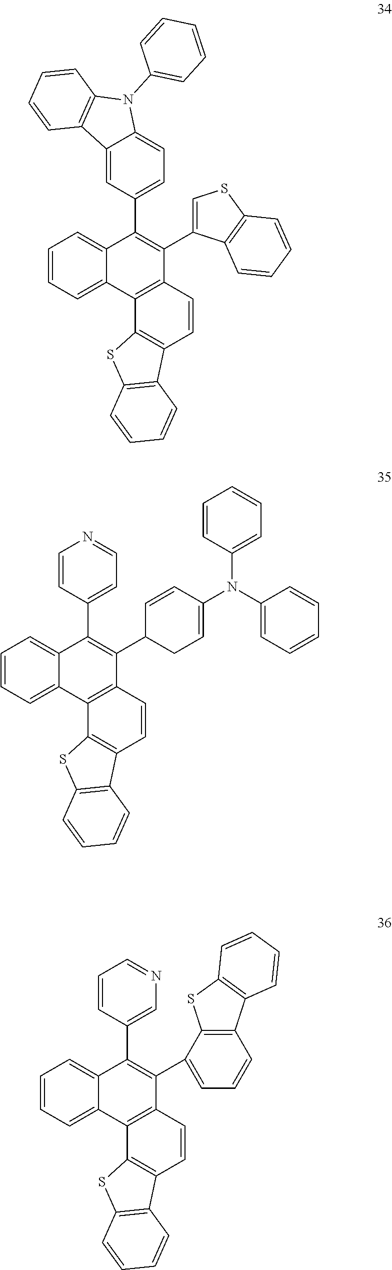 [SMILES: c1ccc(-n2c3ccccc3c3cc(-c4c(-c5csc6ccccc56)c5ccc6c7ccccc7sc6c5c5ccccc45)ccc32)cc1 ; C1=CC(c2c(-c3ccncc3)c3ccccc3c3c2ccc2c4ccccc4sc23)CC=C1N(c1ccccc1)c1ccccc1 ; c1cncc(-c2c(-c3cccc4c3sc3ccccc34)c3ccc4c5ccccc5sc4c3c3ccccc23)c1]

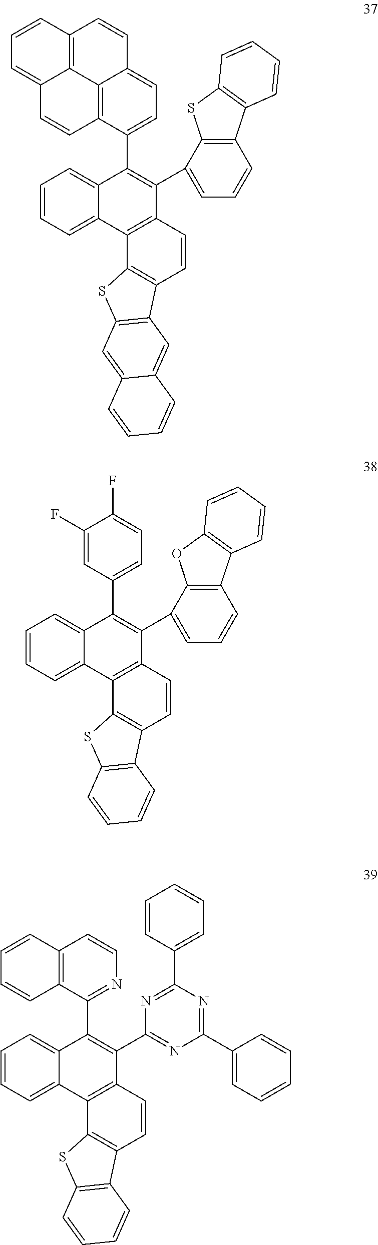 [SMILES: c1ccc2cc3c(cc2c1)sc1c3ccc2c(-c3cccc4c3sc3ccccc34)c(-c3ccc4ccc5cccc6ccc3c4c56)c3ccccc3c21 ; Fc1ccc(-c2c(-c3cccc4c3oc3ccccc34)c3ccc4c5ccccc5sc4c3c3ccccc23)cc1F ; c1ccc(-c2nc(-c3ccccc3)nc(-c3c(-c4nccc5ccccc45)c4ccccc4c4c3ccc3c5ccccc5sc34)n2)cc1]

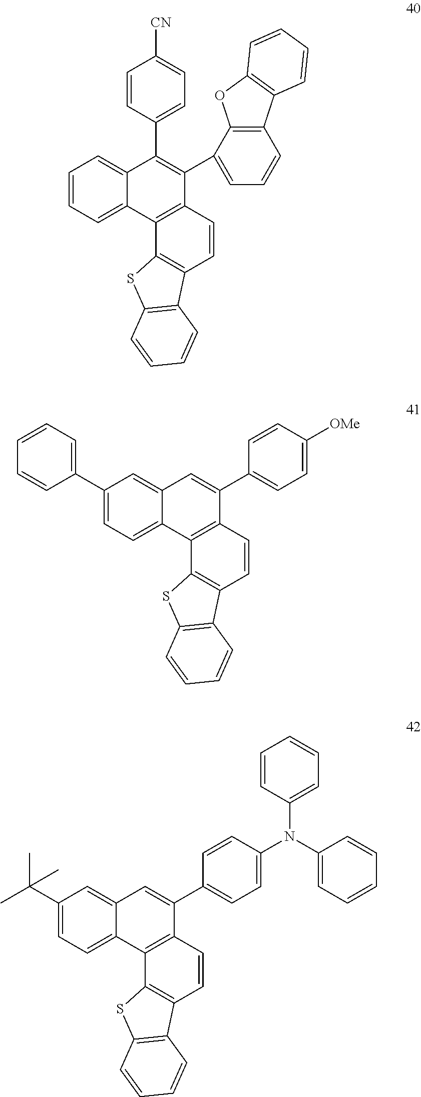 [SMILES: N#Cc1ccc(-c2c(-c3cccc4c3oc3ccccc34)c3ccc4c5ccccc5sc4c3c3ccccc23)cc1 ; COc1ccc(-c2cc3cc(-c4ccccc4)ccc3c3c2ccc2c4ccccc4sc23)cc1 ; CC(C)(C)c1ccc2c(c1)cc(-c1ccc(N(c3ccccc3)c3ccccc3)cc1)c1ccc3c4ccccc4sc3c12]

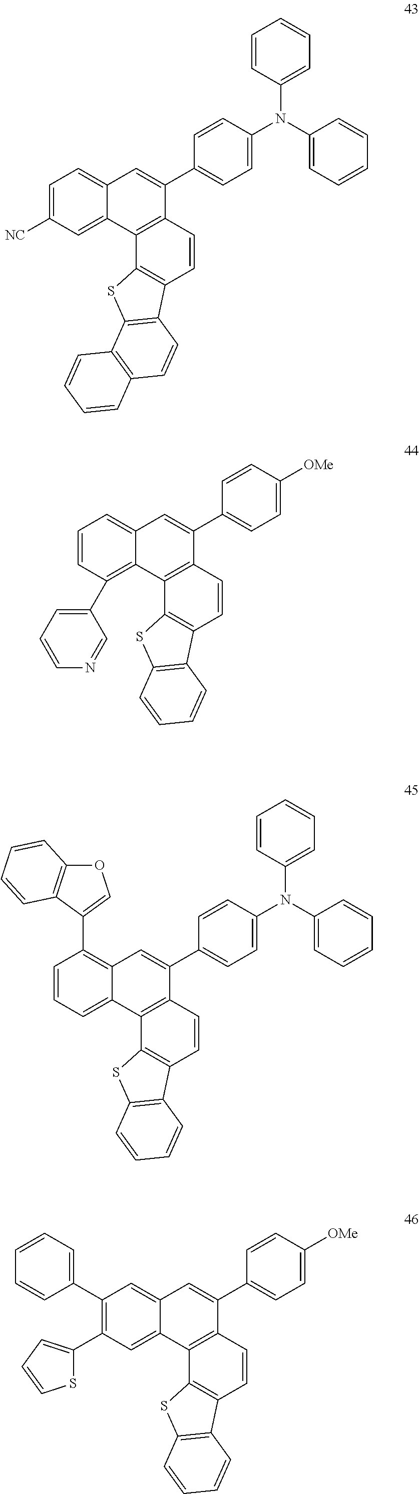 [SMILES: N#Cc1ccc2cc(-c3ccc(N(c4ccccc4)c4ccccc4)cc3)c3ccc4c5ccc6ccccc6c5sc4c3c2c1 ; COc1ccc(-c2cc3cccc(-c4cccnc4)c3c3c2ccc2c4ccccc4sc23)cc1 ; c1ccc(N(c2ccccc2)c2ccc(-c3cc4c(-c5coc6ccccc56)cccc4c4c3ccc3c5ccccc5sc34)cc2)cc1 ; COc1ccc(-c2cc3cc(-c4ccccc4)c(-c4cccs4)cc3c3c2ccc2c4ccccc4sc23)cc1]

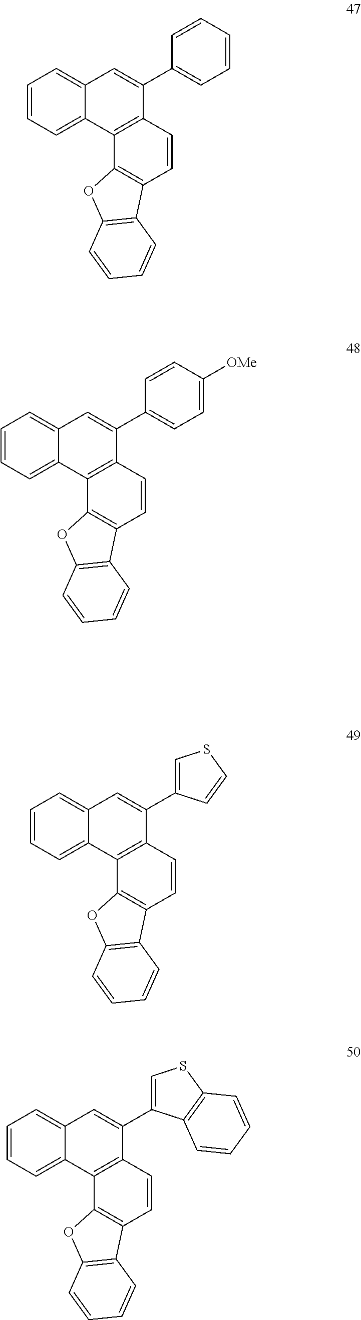 [SMILES: c1ccc(-c2cc3ccccc3c3c2ccc2c4ccccc4oc23)cc1 ; COc1ccc(-c2cc3ccccc3c3c2ccc2c4ccccc4oc23)cc1 ; c1ccc2c(c1)cc(-c1ccsc1)c1ccc3c4ccccc4oc3c12 ; c1ccc2c(c1)cc(-c1csc3ccccc13)c1ccc3c4ccccc4oc3c12]

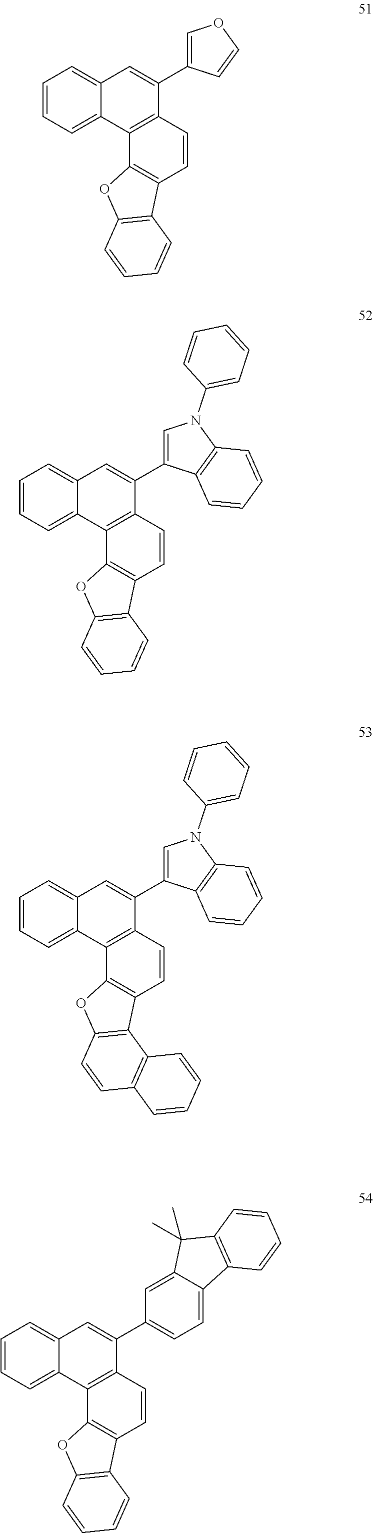 [SMILES: c1ccc2c(c1)cc(-c1ccoc1)c1ccc3c4ccccc4oc3c12 ; c1ccc(-n2cc(-c3cc4ccccc4c4c3ccc3c5ccccc5oc34)c3ccccc32)cc1 ; c1ccc(-n2cc(-c3cc4ccccc4c4c3ccc3c4oc4ccc5ccccc5c43)c3ccccc32)cc1 ; CC1(C)c2ccccc2-c2ccc(-c3cc4ccccc4c4c3ccc3c5ccccc5oc34)cc21]

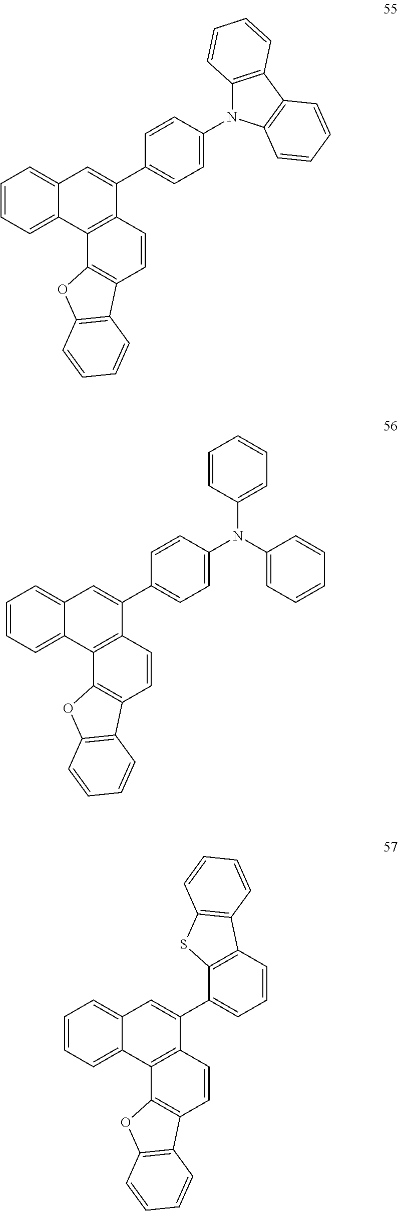 [SMILES: c1ccc2c(c1)cc(-c1ccc(-n3c4ccccc4c4ccccc43)cc1)c1ccc3c4ccccc4oc3c12 ; c1ccc(N(c2ccccc2)c2ccc(-c3cc4ccccc4c4c3ccc3c5ccccc5oc34)cc2)cc1 ; c1ccc2c(c1)cc(-c1cccc3c1sc1ccccc13)c1ccc3c4ccccc4oc3c12]

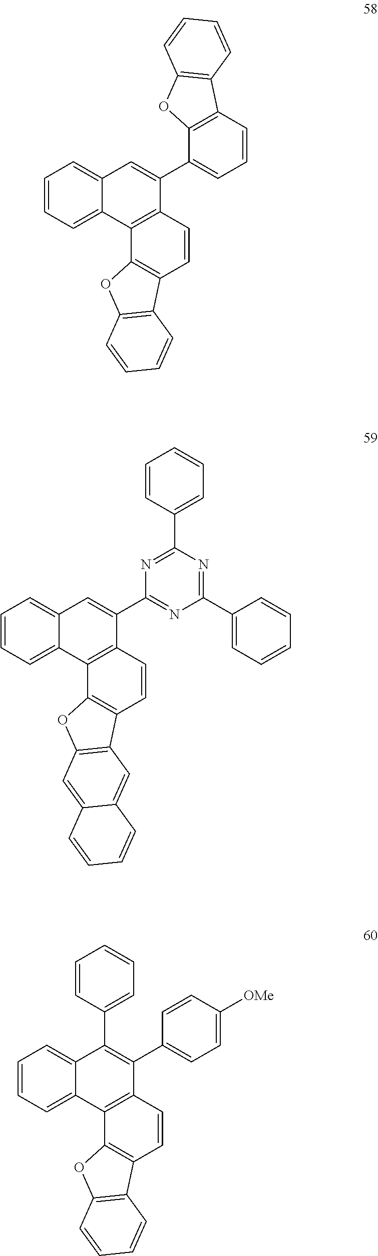 [SMILES: c1ccc2c(c1)cc(-c1cccc3c1oc1ccccc13)c1ccc3c4ccccc4oc3c12 ; c1ccc(-c2nc(-c3ccccc3)nc(-c3cc4ccccc4c4c3ccc3c5cc6ccccc6cc5oc34)n2)cc1 ; COc1ccc(-c2c(-c3ccccc3)c3ccccc3c3c2ccc2c4ccccc4oc23)cc1]

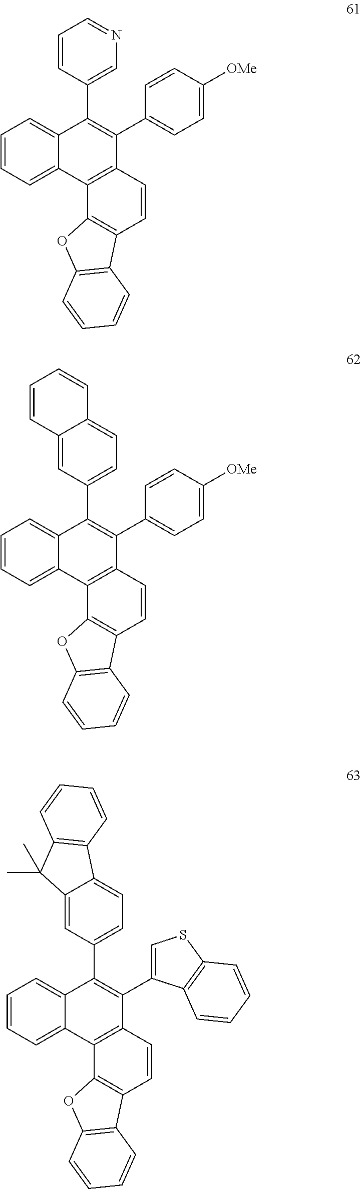 [SMILES: COc1ccc(-c2c(-c3cccnc3)c3ccccc3c3c2ccc2c4ccccc4oc23)cc1 ; COc1ccc(-c2c(-c3ccc4ccccc4c3)c3ccccc3c3c2ccc2c4ccccc4oc23)cc1 ; CC1(C)c2ccccc2-c2ccc(-c3c(-c4csc5ccccc45)c4ccc5c6ccccc6oc5c4c4ccccc34)cc21]

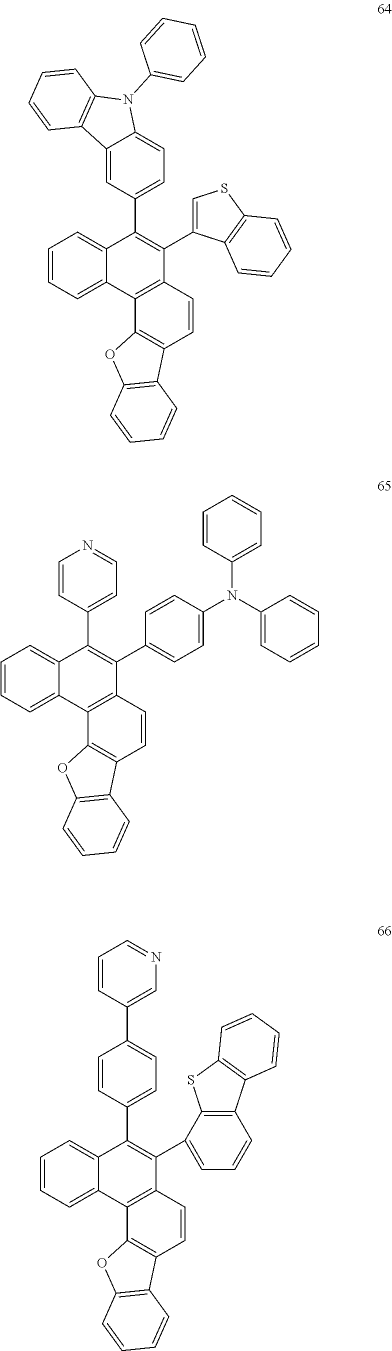 [SMILES: c1ccc(-n2c3ccccc3c3cc(-c4c(-c5csc6ccccc56)c5ccc6c7ccccc7oc6c5c5ccccc45)ccc32)cc1 ; c1ccc(N(c2ccccc2)c2ccc(-c3c(-c4ccncc4)c4ccccc4c4c3ccc3c5ccccc5oc34)cc2)cc1 ; c1cncc(-c2ccc(-c3c(-c4cccc5c4sc4ccccc45)c4ccc5c6ccccc6oc5c4c4ccccc34)cc2)c1]

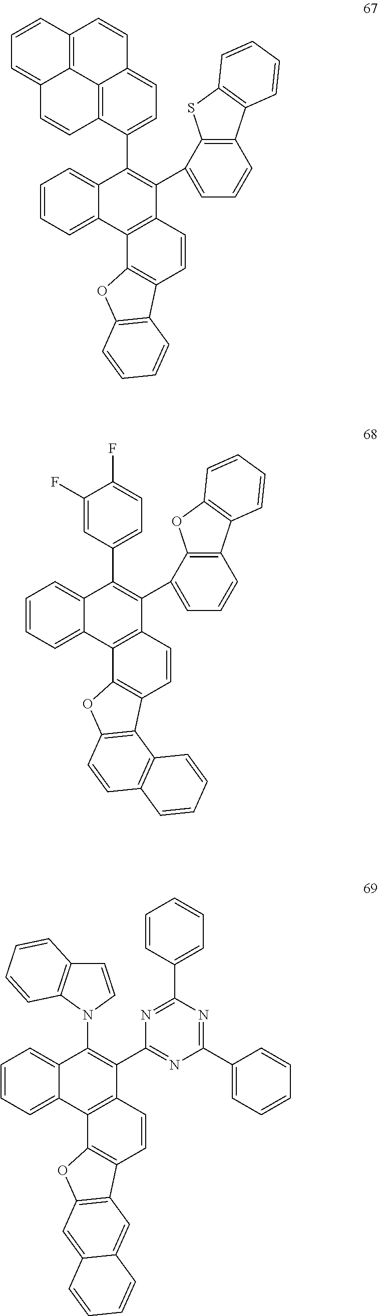 [SMILES: c1cc2ccc3ccc(-c4c(-c5cccc6c5sc5ccccc56)c5ccc6c7ccccc7oc6c5c5ccccc45)c4ccc(c1)c2c34 ; Fc1ccc(-c2c(-c3cccc4c3oc3ccccc34)c3ccc4c(oc5ccc6ccccc6c54)c3c3ccccc23)cc1F ; c1ccc(-c2nc(-c3ccccc3)nc(-c3c(-n4ccc5ccccc54)c4ccccc4c4c3ccc3c5cc6ccccc6cc5oc34)n2)cc1]

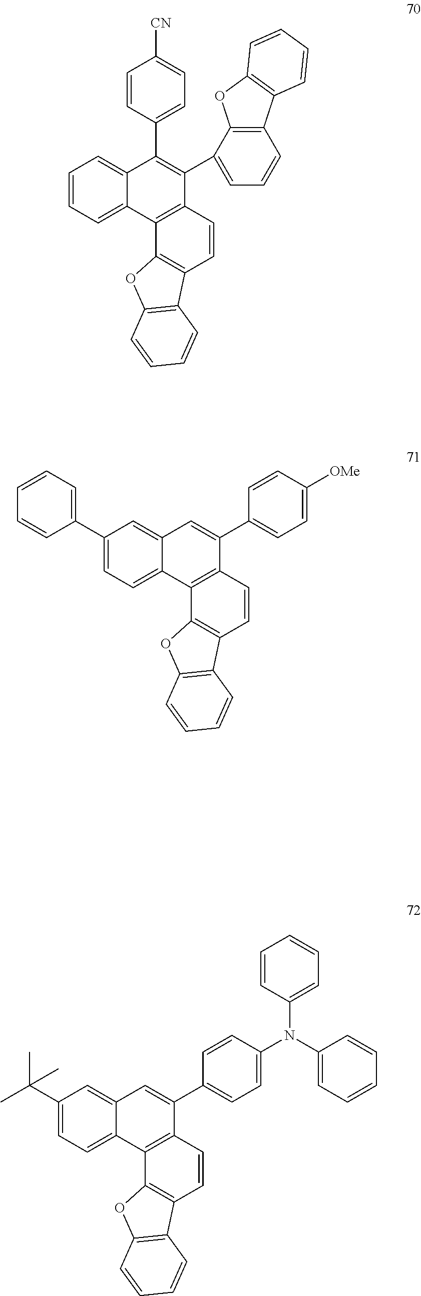 [SMILES: N#Cc1ccc(-c2c(-c3cccc4c3oc3ccccc34)c3ccc4c5ccccc5oc4c3c3ccccc23)cc1 ; COc1ccc(-c2cc3cc(-c4ccccc4)ccc3c3c2ccc2c4ccccc4oc23)cc1 ; CC(C)(C)c1ccc2c(c1)cc(-c1ccc(N(c3ccccc3)c3ccccc3)cc1)c1ccc3c4ccccc4oc3c12]

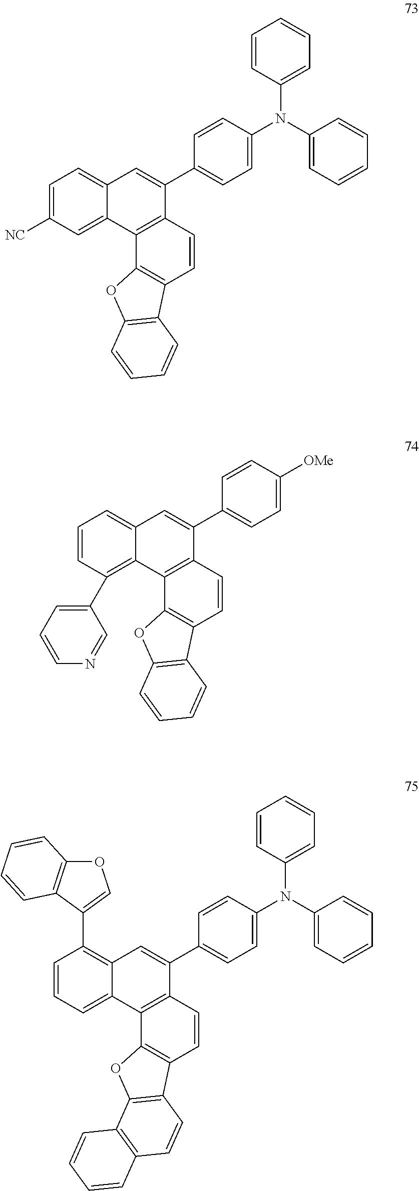 [SMILES: N#Cc1ccc2cc(-c3ccc(N(c4ccccc4)c4ccccc4)cc3)c3ccc4c5ccccc5oc4c3c2c1 ; COc1ccc(-c2cc3cccc(-c4cccnc4)c3c3c2ccc2c4ccccc4oc23)cc1 ; c1ccc(N(c2ccccc2)c2ccc(-c3cc4c(-c5coc6ccccc56)cccc4c4c3ccc3c5ccc6ccccc6c5oc34)cc2)cc1]

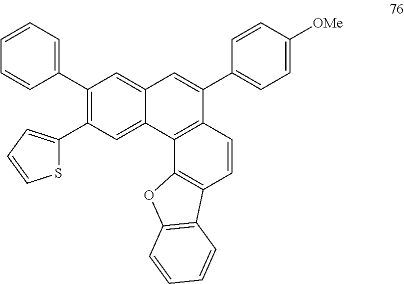 [SMILES: COc1ccc(-c2cc3cc(-c4ccccc4)c(-c4cccs4)cc3c3c2ccc2c4ccccc4oc23)cc1]

The condensed-cyclic compound of Formula 1 as described above may have a structure in which a hetero ring is introduced into molecules and may have high glass transition temperature and/or a high melting point. Thus, when an organic light-emitting diode (OLED) including the condensed-cyclic compound of Formula 1 between a pair of electrodes (anode and cathode) is operated, the OLED may have high heat resistance to Joule's heat generated between organic layers positioned between the pair of electrodes or between one of the organic layers and one of the electrodes.

Examples of the unsubstituted C₁-C₆₀ alkyl group (or C₁-C₆₀ alkyl group) may include methyl, ethyl, propyl, isobutyl, sec-butyl, pentyl, iso-amyl, hexyl, and the like. The substituted C₁-C₆₀ alkyl group may be a group in which at least one hydrogen of the unsubstituted C₁-C₆₀ alkyl group is substituted with deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₆₀ alkyl group; a C₂-C₆₀ alkenyl group; a C₂-C₆₀ alkynyl group; a C₁-C₆₀ alkoxy group; a C₃-C₆₀ cycloalkyl group; a C₅-C₆₀ aryl group; a C₅-C₆₀ aryloxy group; a C₅-C₆₀ arylthio group; a C₁-C₆₀ heteroaryl group; a C₁-C₆₀ alkyl group, a C₂-C₆₀ alkenyl group, a C₂-C₆₀ alkynyl group, a C₁-C₆₀ alkoxy group, a C₃-C₆₀ cycloalkyl group, a C₅-C₆₀ aryl group, a C₅-C₆₀ aryloxy group, a C₅-C₆₀ arylthio group, or a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group; —N(Q₁)(Q₂); or —Si(Q₃)(Q₄)(Q₅) (Q₁ through Q₅ may be each independently a C₃-C₆₀ cycloalkyl group; a C₅-C₆₀ aryl group; a C₅-C₆₀ aryloxy group; a C₅-C₆₀ arylthio group; a C₂-C₆₀ heteroaryl group; and a C₁-C₆₀ alkyl group, a C₂-C₆₀ alkenyl group, a C₂-C₆₀ alkynyl group, and a C₁-C₆₀ alkoxy group, a C₃-C₆₀ cycloalkyl group, a C₅-C₆₀ aryl group, a C₅-C₆₀ aryloxy group, a C₅-C₆₀ arylthio group, or a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group.

The unsubstituted C₁-C₆₀ alkoxy group (or C₁-C₆₀ alkoxy group) may have a formula of —OA (in this regard, A may be the unsubstituted C₁-C₆₀ alkyl group as described above) and examples thereof may include methoxy, ethoxy, isopropyloxy, and the like. At least one hydrogen atom of the unsubstituted C₁-C₆₀ alkoxy group may be substituted with the same substituent as in the substituted C₁-C₆₀ alkyl group described above.

The unsubstituted C₂-C₆₀ alkenyl group (or C₂-C₆₀ alkenyl group) may be interpreted to contain at least one carbon-carbon double bond in the center or at a terminal of the unsubstituted C₂-C₆₀ alkyl group. Examples of the unsubstituted C₂-C₆₀ alkenyl group may include ethenyl, propenyl, butenyl, and the like. At least one hydrogen atom of the unsubstituted C₂-C₆₀ alkenyl group may be substituted with the substituents described with reference to the substituted C₁-C₆₀ alkyl group described above.

The unsubstituted C₂-C₆₀ alkynyl group (or C₂-C₆₀ alkynyl group) may be interpreted to contain at least one carbon-carbon triple bond in the center or at a terminal of the C₂-C₆₀ alkyl group defined above. Examples of the unsubstituted C₂-C₆₀ alkynyl group may include ethynyl, propynyl, and the like. At least one hydrogen atom of the unsubstituted C₂-C₆₀ alkynyl group may be substituted with the substituents described with reference to the substituted C₁-C₆₀ alkyl group described above.

The unsubstituted C₅-C₆₀ aryl group indicates a monovalent group having an aromatic carbocyclic system that has 5 to 60 carbon atoms and at least one aromatic ring and the unsubstituted C₅-C₆₀ arylene group indicates a divalent group having an aromatic carbocyclic system that has 5 to 60 carbon atoms and at least one aromatic ring. If the C₅-C₆₀ aryl group and the C₅-C₆₀ arylene group each independently have two or more aromatic rings, the rings may be fused with each other. At least one hydrogen atom of each of the unsubstituted C₅-C₆₀ aryl group and the unsubstituted C₅-C₆₀ arylene group may be substituted with the substituents described with reference to the C₁-C₆₀ alkyl group.

Examples of the substituted or unsubstituted C₅-C₆₀ aryl group may include, but are not limited to, a phenyl group, a C₁-C₁₀ alkylphenyl group (e.g., an ethylphenyl group), a C₁-C₁₀ alkylbiphenyl group (e.g., an ethylbiphenyl group), a halophenyl group (e.g., an o-, m- and p-fluorophenyl group, and a dichlorophenyl group), a dicyanophenyl group, a trifluoromethoxyphenyl group, an o-, m-, and p-tolyl group, an o-, m- and p-cumenyl group, a mesityl group, a phenoxyphenyl group, an (α,α-dimethylbenzene)phenyl group, a (N,N′-dimethyl)aminophenyl group, a (N,N′-diphenyl)aminophenyl group, a pentalenyl group, an indenyl group, a naphthyl group, a halonaphthyl group (e.g., a fluoronaphthyl group), a C₁-C₁₀ alkylnaphthyl group (e.g., a methylnaphthyl group), a C₁-C₁₀ alkoxynaphthyl group (e.g., a methoxynaphthyl group), an anthracenyl group, an azulenyl group, a heptalenyl group, an acenaphthylenyl group, a phenalenyl group, a fluorenyl group, an anthraquinolyl group, a methylanthryl group, a phenanthryl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl, a pyranthrenyl group, and an ovalenyl group. Examples of the substituted C₅-C₆₀ aryl group may be easily understood with reference to the examples of the unsubstituted C₅-C₆₀ aryl group described above and the substituents of the substituted C₁-C₆₀ alkyl group. Examples of the substituted or unsubstituted C₅-C₆₀ arylene group may be easily understood with reference to the substituted or unsubstituted C₅-C₆₀ aryl group described above.

The unsubstituted heteroaryl group indicates a monovalent group having at least one aromatic ring system including carbon rings and at least one hetero atom selected from the group consisting of N, O, P, and S, and the unsubstituted heteroarylene group indicates a divalent group having at least one aromatic ring system including carbon rings and at least one hetero atom selected from the group consisting of N, O, P, and S. In this regard, if the C₂-C₆₀ heteroaryl group and the C₂-C₆₀ heteroarylene group each independently have two or more aromatic rings, the rings may be fused with each other. At least one hydrogen atom of each of the unsubstituted C₂-C₆₀ heteroaryl group and the unsubstituted C₂-C₆₀ heteroarylene group may be substituted with the same substituents as in the C₁-C₆₀ alkyl group described above.

Examples of the unsubstituted C₂-C₆₀ heteroaryl group may include, but are not limited to, a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group, an isoquinolinyl group, a benzoimidazolyl group, an imidazopyridinyl group, and an imidazopyrimidinyl group. Examples of the unsubstituted C₂-C₆₀ heteroarylene group may be easily understood with reference to the examples of the substituted or unsubstituted C₂-C₆₀ arylene group.

The substituted or unsubstituted C₅-C₆₀ aryloxy group may have a formula of —OA₂, wherein A₂ may be the substituted or unsubstituted C₅-C₆₀ aryl group as described above, and the substituted or unsubstituted C₅-C₆₀ arylthio group may have a formula of —OA₃, wherein A₃ may be the substituted or unsubstituted C₅-C₆₀ aryl group described above.

For example, the condensed-cyclic compound of Formula 1 may be synthesized according to Reaction Scheme 1 below:

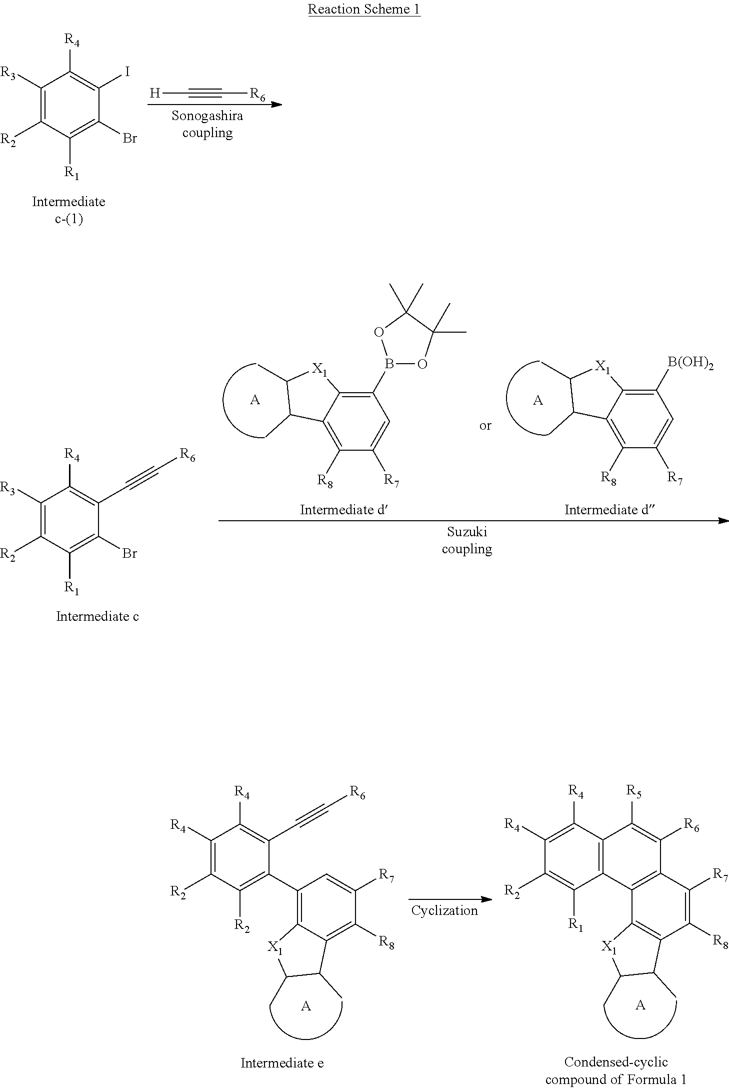

In Reaction Scheme 1 above, X₁, R₁ through R₈, R₁₀, and a ring A are the same as defined above and a detailed description of Formula 1 is already provided above.

According to another embodiment of the present invention, there is provided a method of preparing the condensed-cyclic compound of Formula 1, the method may include reacting Intermediate c-(1) with

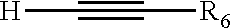

to obtain Intermediate c; reacting the Intermediate c with Intermediate d′ or d″ to obtain Intermediate e; and cyclizing the Intermediate e to obtain the condensed-cyclic compound of Formula 1.

The reacting of the Intermediate c-(1) with

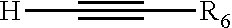

to obtain Intermediate c may be performed by Sonogashira coupling and it may be easily understood by one of ordinary skill in the art with reference to Examples, which will be described later.

The reacting of the Intermediate c with Intermediate d′ or d″ to obtain Intermediate e may be performed by Suzuki coupling and it may be easily understood by one of ordinary skill in the art with reference to Examples, which will be described later.

The cyclizing of the Intermediate e to obtain the condensed-cyclic compound of Formula 1 may be easily understood by one of ordinary skill in the art with reference to Examples, which will be described later.

The

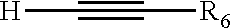

may be synthesized according to Reaction Scheme 1′ below:

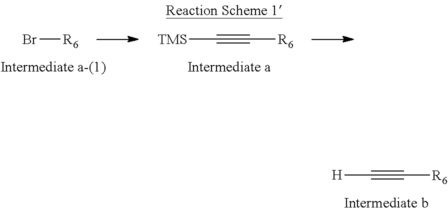

In Reaction Scheme 1′ above, TMS denotes trimethylsilyl.

Reaction Scheme 1′ may be easily understood by one of ordinary skill in the art with reference to Examples, which will be described later.

The condensed-cyclic compound of Formula 1 may be used between a pair of electrodes of an OLED. For example, the condensed-cyclic compound of Formula 1 may be used as a light-emitting material, an electron transporting material, and/or an electron injection material, but is not limited thereto.

According to another embodiment of the present invention, there is provided an OLED including a first electrode, a second electrode facing the first electrode, and a first layer interposed between the first electrode and the second electrode, wherein the first layer includes the condensed-cyclic compound of Formula 1 described above.

The first layer may include at least one of the condensed-cyclic compounds of Formula 1.

The expression “the first layer may include at least one of the condensed-cyclic compounds of Formula 1” as used herein means that the first layer includes one of the condensed-cyclic compounds represented by Formula 1 above or at least two different compounds selected from the condensed-cyclic compounds represented by Formula 1 above.

When the first layer includes at least two different compounds selected from the condensed-cyclic compounds represented by Formula 1 above, the at least two different compounds may be included in a single layer in a mixed form (e.g., at least two condensed-cyclic compounds of Formula 1 may be included in an emission layer (EML)) or may be each independently included in different layers (e.g., one of the at least two condensed-cyclic compounds of Formula 1 is included in an EML and another thereof is included in an electron transport layer (ETL)).

For example, an OLED manufactured according to Example 1, which will be described later, includes only Compound 10 (acting as a green phosphorescent host) as the condensed-cyclic compound of Formula 1. On the other hand, an OLED manufactured according to Example 7, which will be described later, includes Compound 7 (acting as a red phosphorescent host) and Compound 36 (acting as an electron transporting material) as the condensed-cyclic compound of Formula 1. In this regard, Compound 7 is included in an EML and Compound 36 is included in an ETL.

The first layer may include at least one of a hole injection layer (HIL), a hole transport layer (HTL), a functional layer having hole injection and hole transport abilities, an electron blocking layer (EBL), an EML, a hole blocking layer (HBL), an ETL, an electron injection layer (EIL), and a functional layer having electron transport and electron injection abilities.

The term “first layer” used herein refers to a single layer or multiple layers interposed between the first electrode and the second electrode.

For example, the first layer includes an EML including the condensed-cyclic compound of Formula 1. In other words, the condensed-cyclic compound of Formula 1 may be used as a light-emitting material. In this regard, the EML may further include a phosphorescent dopant and the condensed-cyclic compound of Formula 1 included in the EML may serve as a phosphorescent host. The phosphorescent dopant may be iridium (Ir), platinum (Pt), osmium (Os), rhenium (Re), titanium (Ti), zirconium (Zr), hafnium (Hf), or an organic metal complex including at least two of these materials, but is not limited thereto.

The EML may be a red, green or blue EML. For example, the EML may be a green EML. In this regard, the condensed-cyclic compound of Formula 1 is used as a green phosphorescent host and/or a red phosphorescent host, whereby an OLED including the condensed-cyclic compound of Formula 1 may have high efficiency, brightness and color purity and long lifetime.

In addition, the first layer may include an ETL including the condensed-cyclic compound of Formula 1 (refer to Example 5 below). In this regard, the ETL may further include a metal-containing compound.

Also, the first layer may include both the EML and the ETL, wherein each of the EML and the ETL may include the condensed-cyclic compound of Formula 1. In this regard, the condensed-cyclic compound of Formula 1 included in the EML may be different from the condensed-cyclic compound of Formula 1 included in the ETL (refer to Example 7).

In addition, the first layer may include at least one of the HIL, the HTL, and the functional layer having hole injection and hole transport abilities. In this regard, the at least one of the HIL, the HTL, and the functional layer having hole injection and hole transport abilities may further include a charge-generating material, in addition to a general hole injection material, a general hole transporting material, and a general material having hole injection and hole transport abilities.

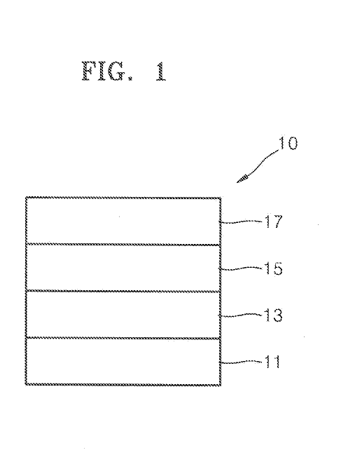

FIG. 1 is a schematic cross-sectional view of an OLED 10 according to an embodiment of the present invention. Hereinafter, structure and manufacturing method of an OLED will be described in more detail with reference to FIG. 1.

The OLED 10 includes a substrate 11, and a first electrode 13, a first layer 15, and a second electrode 17 that are sequentially formed on the substrate 11.

The substrate 11 may be a substrate used in a general OLED, and may be a glass substrate or a transparent plastic substrate having excellent mechanical strength, thermal stability, transparency, surface smoothness, ease of handling, and waterproofness.

The first electrode 13 may be formed by applying a first electrode material on the substrate 11 by deposition or sputtering. When the first electrode 13 is an anode, the first electrode material may be selected from materials having a high work function so as to facilitate hole injection. The first electrode 13 may be a reflective electrode or a transparent electrode. Examples of the first electrode material may include indium-tin oxide (ITO), Indium-zinc-oxide (IZO), tin oxide (SnO₂), and zinc oxide (ZnO). Also, when magnesium (Mg), aluminum (Al), aluminum-lithium (Al—Li), calcium (Ca), magnesium-indium (Mg—In), or magnesium-silver (Mg—Ag) is used as the first electrode material, the first electrode 13 may be formed as a reflective electrode.

The first electrode 13 may be formed as a single layer or have a multi-layered structure having at least two layers. For example, the first electrode 13 may have a three-layered structure, e.g., ITO/Ag/ITO, but is not limited thereto.

The first layer 15 is formed on the first electrode 13.

The first layer 15 may include a HIL, a HTL, an EML, a HBL, an ETL, and an EIL.

The HIL may be formed on the first electrode 13 by using various methods such as vacuum deposition, spin coating, casting, or LB deposition.

When the HIL is formed by vacuum deposition, the deposition conditions may vary according to a compound used as a material for forming the HIL, a structure of a desired HIL, and thermal characteristics. For example, the deposition condition may be, but is not limited to, a deposition temperature of about 100 to about 500° C., a degree of vacuum of about 10⁻⁸ to about 10⁻³ torr, and a deposition speed of about 0.01 to about 100 Å/sec.

When the HIL is formed by spin coating, the coating condition may vary according to a compound used as a material for forming the HIL, a structure of a desired HIL, and thermal characteristics. For example, the coating condition may be, but is not limited to, a coating speed of about 2,000 to about 5,000 rpm and a heat treatment temperature for removing a solvent after coating of about 80 to about 200° C.

The material for forming the HIL may be a known hole injection material. Examples of the known hole injection material may include, but are not limited to, N,N′-diphenyl-N,N′-bis-[4-(phenyl-m-tolyl-amino)-phenyl]-biphenyl-4,4′-diamine (DNTPD), a phthalocyanine compound such as copper phthalocyanine, 4,4′,4″-tris(3-methylphenylphenylamino)triphenylamine (m-MTDATA), N,N′-di(1-naphthyl)-N,N′-diphenylbenzidine (NPB), TDATA, 2-TNATA, polyaniline/dodecylbenzenesulfonic acid (PANI/DBSA), poly(3,4-ethylenedioxythiophene)/poly(4-styrenesulfonate) (PEDOT/PSS), polyaniline/camphor sulfonicacid (PANI/CSA), and polyaniline/poly(4-styrenesulfonate) (PANI/PSS).

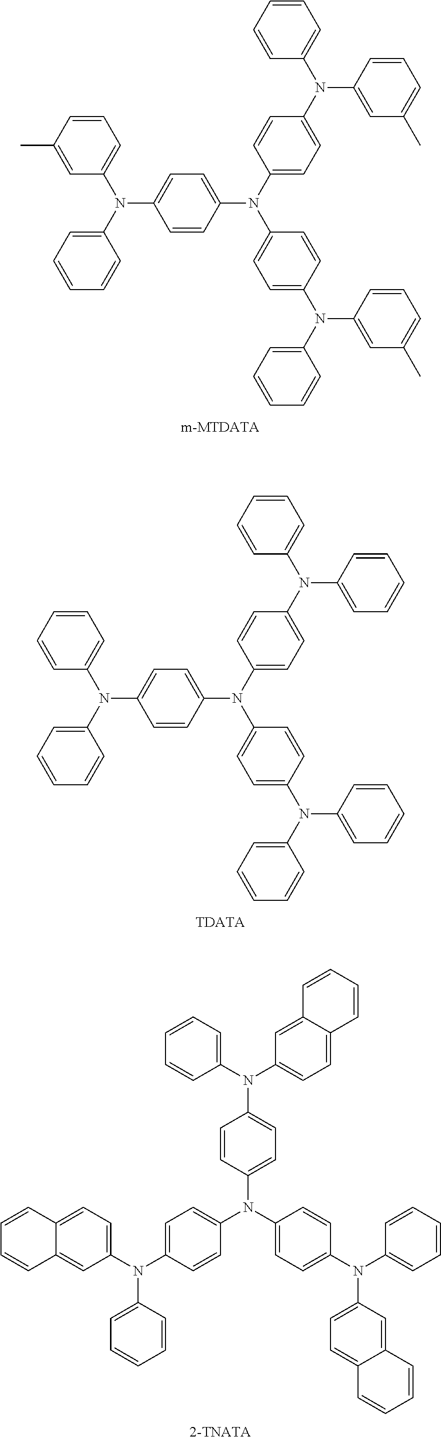

The thickness of the HIL may be in the range of about 100 Å to about 10,000 Å. In some embodiments, the thickness of the HIL may be in the range of about 100 Å to about 1,000 Å. When the thickness of the HIL is within these ranges, satisfactory hole injection properties may be obtained without a substantial increase in driving voltage.

Next, an HTL may be formed on the HIL by using various methods such as vacuum deposition, spin coating, casting, or LB deposition. When the HTL is formed by vacuum deposition or spin coating, the deposition and coating conditions may vary according to a used compound. However, in general, the deposition and coating conditions may be almost the same as the condition for forming the HIL.

A material for forming the HTL may be a known hole transporting material. Examples of the known hole transporting material may include, but are not limited to, carbazole derivatives such as N-phenylcarbazole and polyvinylcarbazole, N,N′-bis(3-methylphenyl)-N,N′-diphenyl-[1,1-biphenyl]-4,4′-diamine (TPD), 4,4′,4″-tris(N-carbazolyl)triphenylamine (TCTA), and N,N′-di(1-naphthyl)-N,N′-diphenylbenzidine (NPB).

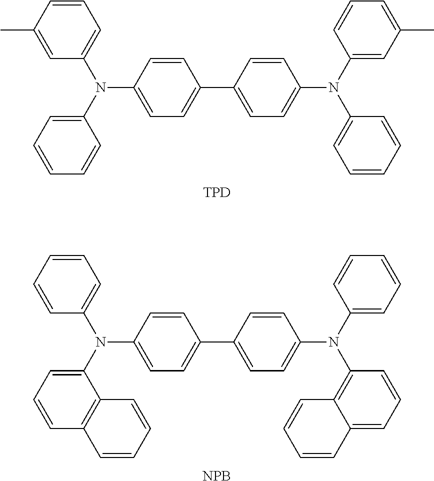

The thickness of the HTL may be in the range of about 50 Å to about 2,000 Å. In some embodiments, the thickness of the HTL may be in the range of about 100 Å to about 1,500 Å. When the thickness of the HTL is within these ranges, satisfactory hole transport properties may be obtained without a substantial increase in driving voltage.

In addition, the functional layer having hole injection and hole transport abilities may be formed instead of the HIL and the HTL. A material for forming the functional layer having hole injection and hole transport abilities may be selected from known materials.

At least one of the HIL, the HTL, and the functional layer having hole injection and hole transport abilities may further include a charge-generating material so as to increase the conductivity of the layers, in addition to the known hole injection material, the known hole transport material and/or the material for forming the functional layer having hole injection and hole transport abilities.

The charge-generating material may be, for example, a p-dopant. Examples of the p-dopant may include, but are not limited to, quinone derivatives such as tetra-cyanoquinodimethane (TCNQ) and 2,3,5,6-tetrafluoro-tetracyano-1,4-benzoquinodimethane (F4TCNQ); metal oxides such as an tungsten oxide and a molybdenum oxide; and cyano-containing compounds such as Compound 200 below and the like.

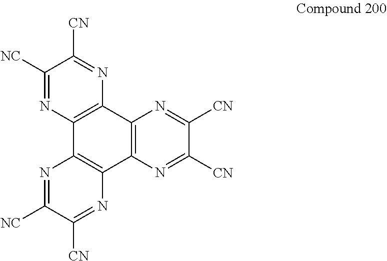 [SMILES: N#Cc1nc2c3nc(C#N)c(C#N)nc3c3nc(C#N)c(C#N)nc3c2nc1C#N]

When the HIL, the HTL or the functional layer having hole injection and hole transport abilities further include the charge-generating material, the charge-generating material may be homogeneously or inhomogeneously dispersed in these layers.

An EML may be formed on the HTL or the functional layer having hole injection and hole transport abilities by vacuum deposition, spin coating, casting, or LB deposition. When the EML is formed by vacuum deposition or spin coating, the deposition and coating conditions may vary according to a used compound. However, in general, the condition may be almost the same as the condition for forming the HIL.

A material for forming the EML may be at least one of the condensed-cyclic compounds of Formula 1 and a known light-emitting material (host and/or dopant). For example, the EML may include, as a host, the condensed-cyclic compound of Formula 1 and a known host (i.e., co-host). In addition, the EML may include the condensed-cyclic compound of Formula 1 and a known phosphorescent dopant. In this regard, the condensed-cyclic compound of Formula 1 may act as a phosphorescent host.

Examples of the known host may include, but are not limited to, Tris(8-hydroxyquinolinato)aluminium (Alq3), 4,4′-N,N′-dicabazole-biphenyl (CBP), poly(n-vinylcabazole) (PVK), 9,10-di(naphthalene-2-yl)anthracene (ADN), TCTA, 1,3,5-tris(N-phenylbenzimidazole-2-yl)benzene (TPBI), 3-tert-butyl-9,10-di(naphth-2-yl) anthracene (TBADN), E3, and distyrylarylene (DSA).

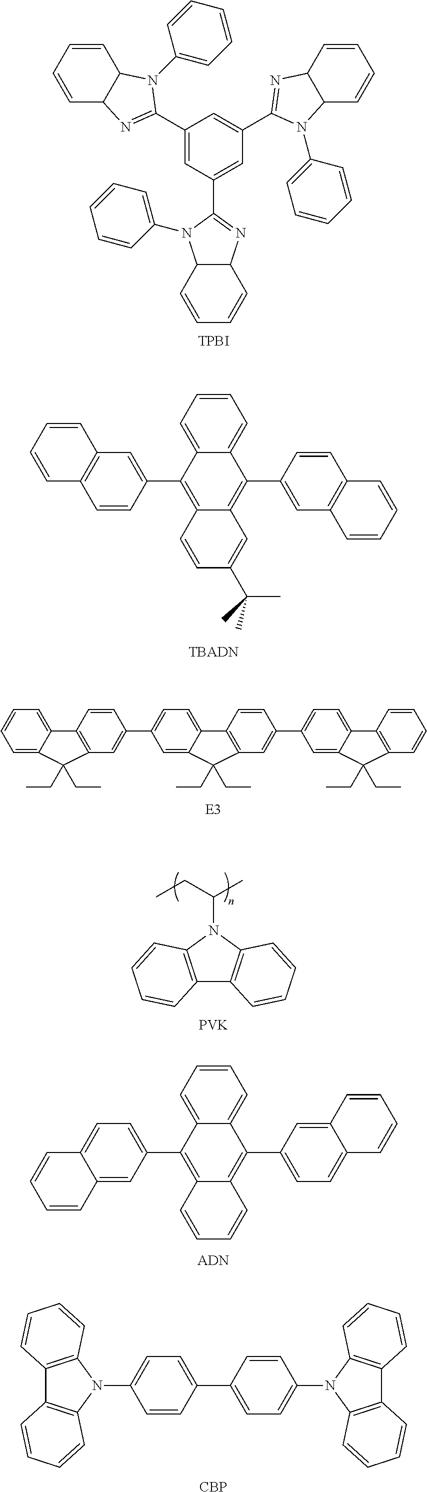

The dopant may be at least one of a fluorescent dopant and a phosphorescent dopant.

Examples of known red dopants may include, but are not limited to, PtOEP, Ir(piq)₃, and Btp₂Ir(acac).

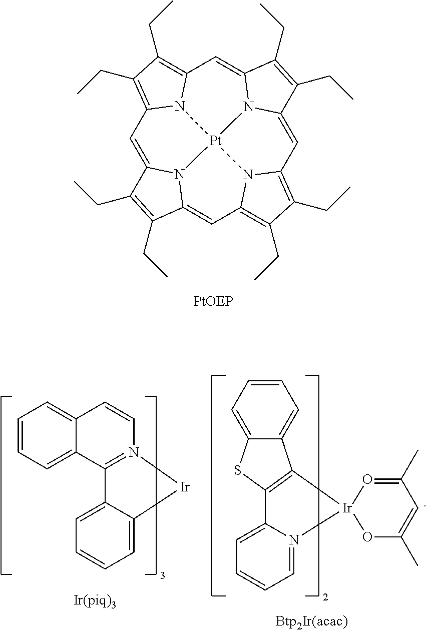

Examples of known green dopants may include, but are not limited to, Ir(ppy)₃ (ppy=phenylpyridine), Ir(ppy)₂(acac), Ir(mpyp)₃, and C545T.

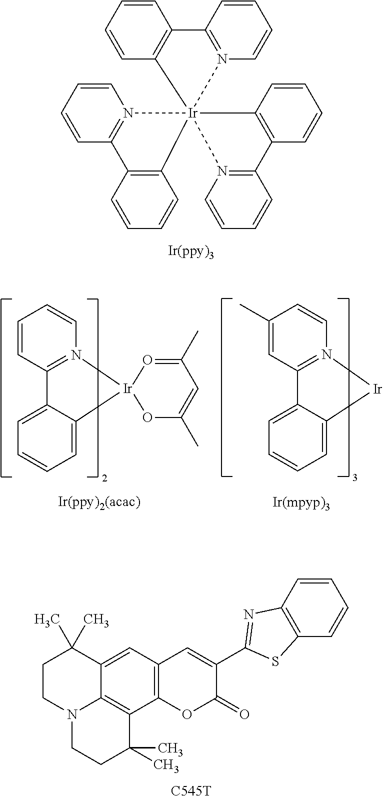

Examples of known blue dopants may include, but are not limited to, F₂Irpic, (F₂ppy)₂Ir(tmd), Ir(dfppz)₃, ter-fluorene, 4,4′-bis(4-diphenylaminostyryl)biphenyl (DPAVBi), 2,5,8,11-tetra-tert-butylperylene (TBPe), and DPVBi.

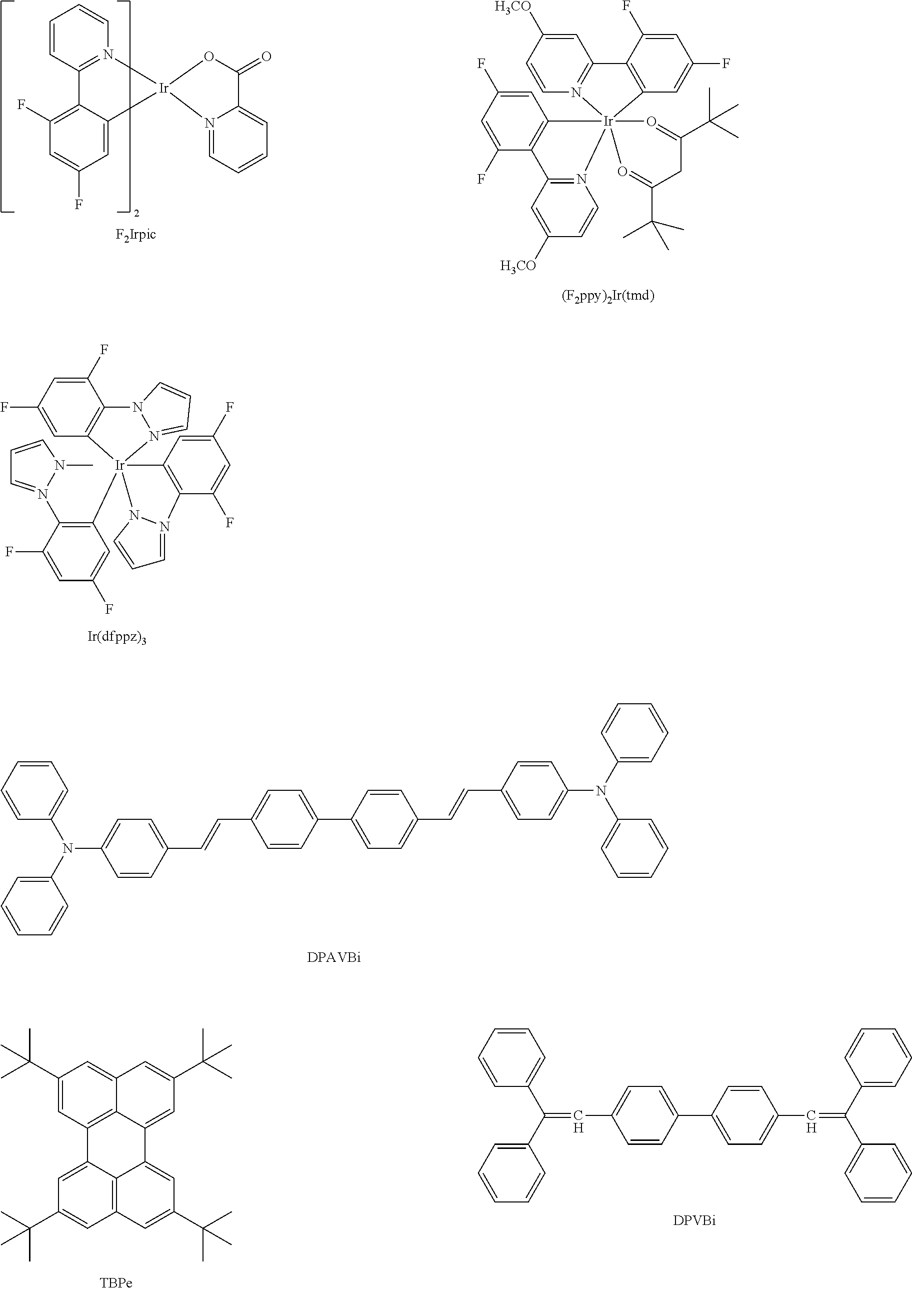

The amount of the dopant in the EML may be generally in the range of about 0.01 to about 15 parts by weight based on 100 parts by weight of the host, but is not limited thereto.

The thickness of the EML may be in the range of about 100 Å to about 1,000 Å. In some embodiments, the thickness of the EML may be in the range of about 200 Å to about 600 Å. When the thickness of the EML is within these ranges, excellent luminescent properties may be obtained without a substantial increase in driving voltage.

When the phosphorescent dopant is included in the EML, a HBL may be formed between the ETL and the EML by vacuum deposition, spin coating, casting or LB deposition so as to prevent triplet excitons or holes from being diffused to the ETL. When the HBL is formed by vacuum deposition or spin coating, the conditions thereof may vary according to a used compound. However, in general, the deposition and coating conditions may be almost the same as the condition for forming the HIL. The HBL may include a known hole blocking material. Examples of the known hole blocking material may include an oxadiazole derivative, a triazole derivative, and a phenanthroline derivative. For example, BCP may be used as a hole blocking material.

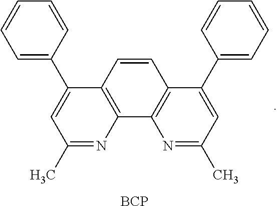 [SMILES: Cc1cc(-c2ccccc2)c2ccc3c(-c4ccccc4)cc(C)nc3c2n1 ; "BCP"]

The thickness of the HBL may be in the range of about 20 Å to about 1,000 Å. In some embodiments, the thickness of the HBL may be in the range of about 30 Å to about 300 Å. When the thickness of the HBL is within these ranges, excellent hole blocking properties may be obtained without a substantial increase in driving voltage.

Next, the ETL may be formed using various methods such as vacuum deposition, spin coating, or casting. When the ETL is formed by vacuum deposition or spin coating, the deposition and coating conditions may vary according to a used compound. However, in general, the deposition and coating conditions may be almost the same as the condition for forming the HIL.

A material for forming the ETL may be a known electron transporting material or the condensed-cyclic compound of Formula 1 that stably transports electrons injected from a cathode. Examples of the known electron transporting material may include, but are not limited to, a quinoline derivative such as tris(8-quinolinolate)aluminum (Alq₃), TAZ, Balq, beryllium bis(benzoquinolin-10-olate (Bebq₂), ADN, and known materials such as Compound 201 and Compound 202 below.

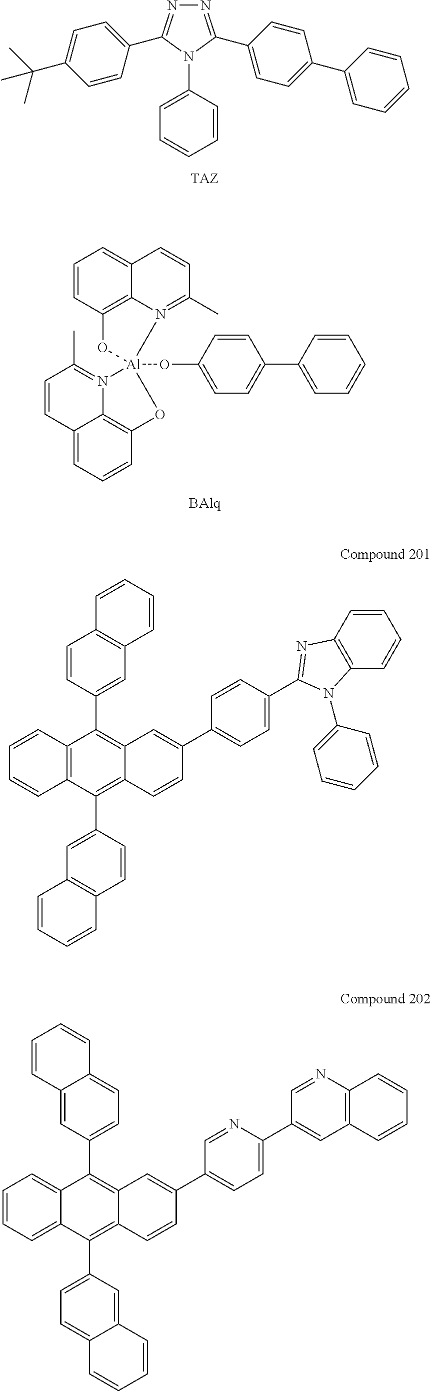

The thickness of the ETL may be in the range of about 100 Å to about 1,000 Å. In some embodiments, the thickness of the ETL may be in the range of about 150 Å to about 500 Å. When the thickness of the ETL is within these ranges, satisfactory electron transport properties may be obtained without a substantial increase in driving voltage.

In addition, the ETL may further include a metal-containing material, in addition to a known electron transporting organic compound or the condensed-cyclic compound of Formula 1.

The metal-containing material may include a Li-complex. Examples of the Li-complex may include lithium quinolate (LiQ) and Compound 203 below:

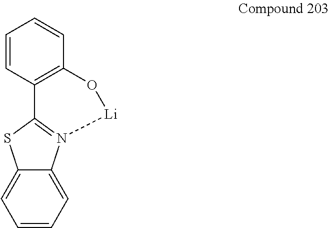

Also, the EIL, which facilitates electron injection from a cathode, may be formed on the ETL, and a material for forming the EIL is not particularly limited.

The material for forming the EIL may include a known material for forming an EIL, such as LiF, NaCl, CsF, Li₂O, or BaO. The deposition condition of the EIL may vary according a used compound. However, in general, the condition may be almost the same as the condition for forming the HIL.

The thickness of the EIL may be in the range of about 1 Å to about 100 Å. In some embodiments, the thickness of the EIL may be in the range of about 3 Å to about 90 Å. When the thickness of the EIL is within these ranges, satisfactory electron injection properties may be obtained without a substantial increase in driving voltage.

The second electrode 17 is formed on the first layer 15. The second electrode 17 may be a cathode, which is an electron injection electrode. In this regard, a metal for forming the second electrode 17 may include a metal having low work function, such as metal, an alloy, an electric conducting compound, and a mixture thereof. In particular, the second electrode 17 may be formed as a thin film by using lithium (Li), magnesium (Mg), aluminum (Al), aluminum-lithium (Al—Li), calcium (Ca), magnesium-indium (Mg—In), or magnesium-silver (Mg—Ag), thus being transparent. In order to obtain a top-emission type organic light-emitting diode, the second electrode 17 may be formed as a transparent electrode by using ITO or IZO.

An OLED according to an embodiment of the present invention will now be described in great detail with reference to the following Examples. These Examples are for illustrative purposes only and are not intended to limit the scope of the invention.

### EXAMPLES

### Synthesis Example 1

### Synthesis of Compound 7

Compound 7 was synthesized according to Reaction Scheme 2 below:

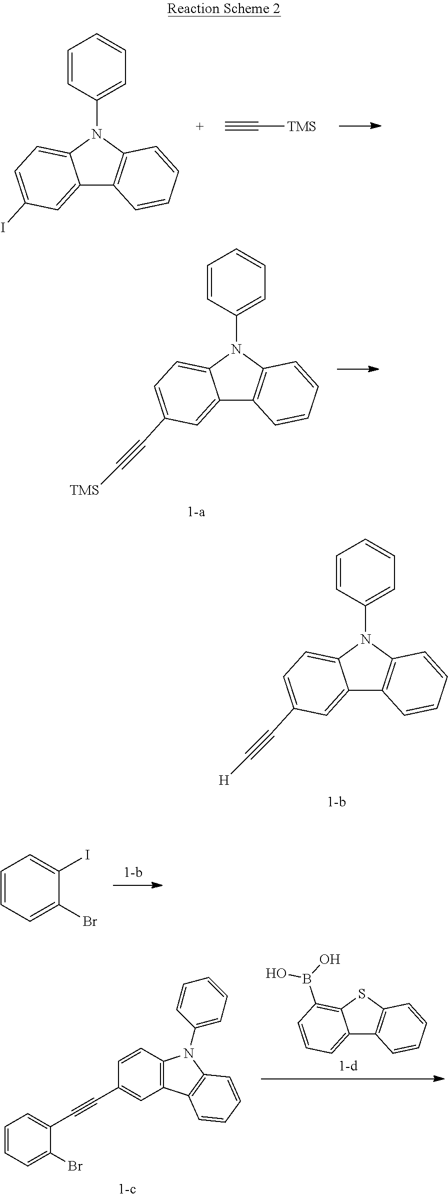

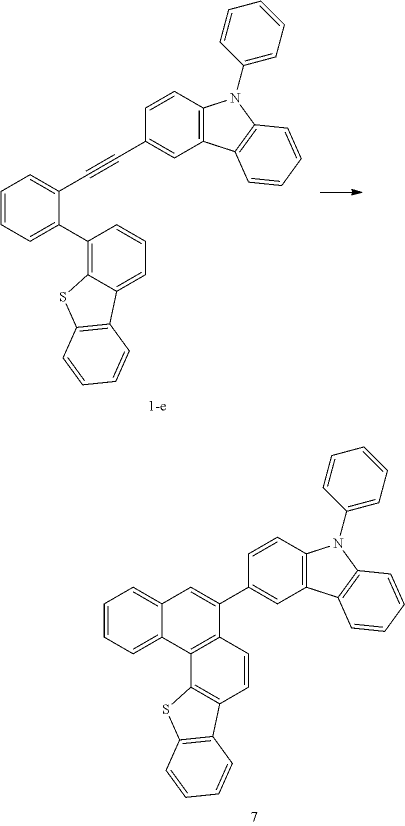 [SMILES: C(#Cc1ccccc1-c1cccc2c1sc1ccccc12)c1ccc2c(c1)c1ccccc1n2-c1ccccc1 ; c1ccc(-n2c3ccccc3c3cc(-c4cc5ccccc5c5c4ccc4c6ccccc6sc45)ccc32)cc1]

### Synthesis of Intermediate 1-a

2.0 g of 3-iodo-9-phenyl-9H-carbazole, 2.8 g (0.04 eq) of tetrakis(triphenylphosphin)palladium (Pd(PPh₃)₄), and 914.0 mg (0.08 eq) of CuI were added to a reactor and the reactor is maintained in vacuum and then in a N₂ atmosphere. Subsequently, 200 ml of tetrahydrofuran (THF) was added to the mixture and the resulting mixture was stirred. Then, 10 ml (1.2 eq) of triethylamine and 10.0 g (1.2 eq) of TMS-acetylene were slowly dropped to the stirred mixture and the resultant mixture was then stirred at room temperature for 2 hours in a N₂ atmosphere. Thereafter, a solvent was removed therefrom by using a rotary evaporator and the reaction product was extracted twice with 200 ml of diethylether (Et₂O) and 150 ml of water. The obtained organic layer was dried with magnesium sulfate and a solvent was evaporated therefrom to obtain a crude product. The crude product was purified with silicagel column chromatography to obtain 20 g of Intermediate 1-a (yield 99%). The obtained compound was confirmed by liquid chromatography-mass spectrometry (LC-MS).

C₂₃H₂₁N₁Si₁: M+339.14

### Synthesis of Intermediate 1-b

4.2 g of Intermediate 1-a was dissolved in 50 ml of THF, 30 ml (3.0 eq) of tetrabutylammonium fluoride (1.0 M in THF) was added to the mixture, and the resulting mixture was then stirred for 30 minutes. Subsequently, 50 ml of water was added to the obtained reaction solution and the reaction solution was extracted three times with 50 ml of ethylether. The obtained organic layer was dried with magnesium sulfate and a solvent was evaporated therefrom to obtain a crude product. The crude product was purified with silicagel column chromatography to obtain 3.5 g of Intermediate 1-b (yield 95%). The obtained compound was confirmed by LC-MS.

C₂₀H₁₃N₁: M+267.10

### Synthesis of Intermediate 1-c

4.4 g (1.2 eq) of 2-bromoiodobenzene, 600 mg (0.04 eq) of Pd(PPh₃)₄, and 200 mg (0.08 eq) of CuI were added to a reactor and the reactor is maintained in vacuum and then in a N₂ atmosphere. Subsequently, 50 ml of THF was added to the mixture and the resulting mixture was stirred. Then, 2.2 ml (1.2 eq) of triethylamine and 3.5 g (1.0 eq) of Intermediate 1-b were slowly dropped to the stirred mixture and the resultant mixture was then stirred at room temperature for 2 hours in a N₂ atmosphere. Thereafter, a solvent was removed therefrom by using a rotary evaporator, 50 ml of water was added to the reaction product, and the reaction product was extracted three times with 50 ml of diethylether (Et₂O). The obtained organic layer was dried with magnesium sulfate and a solvent was evaporated therefrom to obtain a crude product. The crude product was purified with silicagel column chromatography to obtain 3.0 g of Intermediate 1-c (yield 55%). The obtained compound was confirmed by LC-MS.

C₂₆H₁₆Br₁N₁: M+421.05

### Synthesis of Intermediate 1-e

3.0 g of Intermediate 1-c, 1.94 g (1.2 eq) of Intermediate 1-d, 410 mg (0.05 eq) of Pd(PPh₃)₄, and 4.9 g (5.0 eq) of K₂CO₃ were mixed with 50 ml of THF and 15 ml of distilled water, the temperature of the mixed solution was raised to 120° C., and the mixed solution was then stirred by reflux for 24 hours. Subsequently, 1.2 g (0.74 eq) of Intermediate 1-d was added thereto and the resultant mixture was stirred at 120° C. The obtained reaction product was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 3.0 g of Intermediate 1-e (yield 80%). The obtained compound was confirmed by LC-MS.

C₃₃H₂₃N₁S₁: M+525.16

### Synthesis of Compound 7

3.0 g of Intermediate 1-e was mixed with 50 ml of methylene chloride (MC), 8.0 ml (20.0 eq) of trifluoroacetic acid was slowly dropped to the mixture, and the mixture was stirred at room temperature for 1 hour. After the reaction was terminated, the resultant solution was extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 2.7 g of Compound 7 (yield 90%). The obtained compound was confirmed by LC-MS and nuclear magnetic resonance (NMR).

C₃₃H₂₃N₁S₁: M+525.16

1H NMR (CDCl₃, 400 MHz) δ (ppm) 8.93 (d, 1H), 8.12 (m, 2H), 7.93-7.77 (m, 7H), 7.55-7.30 (m, 11H), 7.08-7.00 (m, 2H)

### Synthesis Example 2

### Synthesis of Compound 66

Compound 66 was synthesized according to Reaction Scheme 3 below:

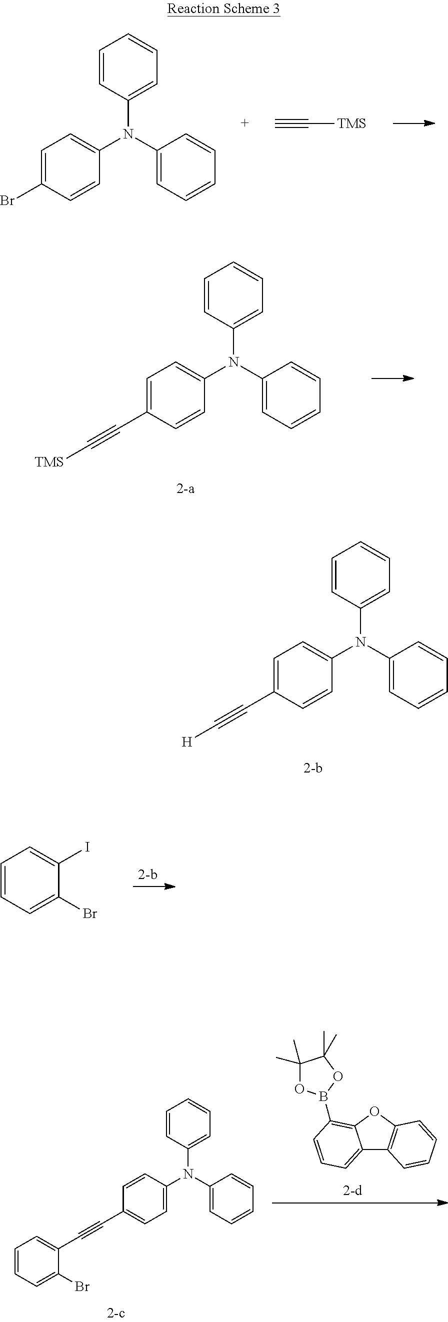

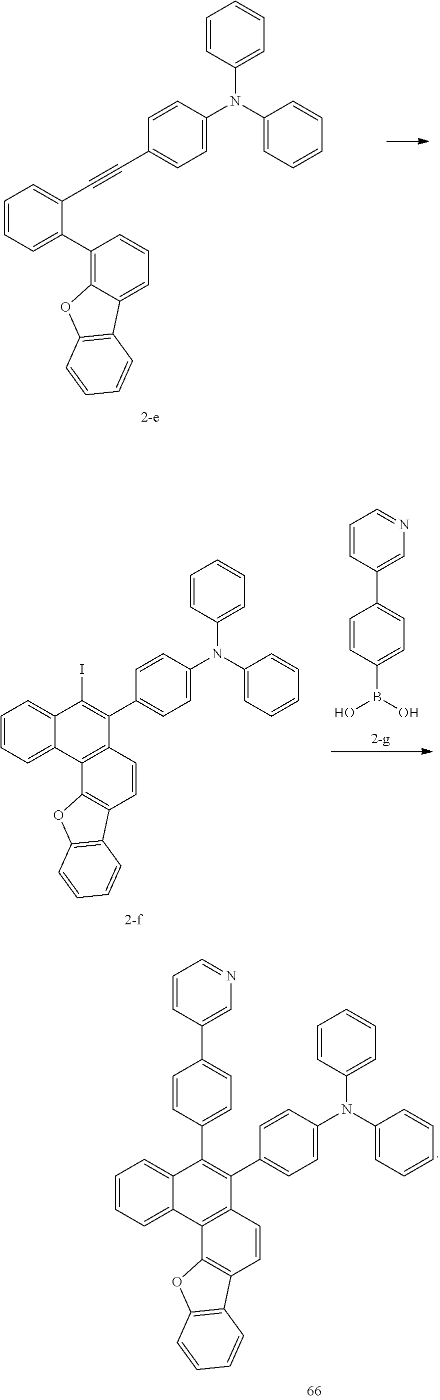 [SMILES: C(#Cc1ccccc1-c1cccc2c1oc1ccccc12)c1ccc(N(c2ccccc2)c2ccccc2)cc1 ; Ic1c(-c2ccc(N(c3ccccc3)c3ccccc3)cc2)c2ccc3c4ccccc4oc3c2c2ccccc12 ; OB(O)c1ccc(-c2cccnc2)cc1 ; c1ccc(N(c2ccccc2)c2ccc(-c3c(-c4ccc(-c5cccnc5)cc4)c4ccccc4c4c3ccc3c5ccccc5oc34)cc2)cc1]

### Synthesis of Intermediate 2-a

10.0 g of (4-bromo-phenyl)-diphenyl-amine, 0.60 mg (0.04 eq) of bis(chloro(triphenylphosphine))palladium (PdCl₂(PPh₃)₂), and 470 mg (0.08 eq) of CuI were added to a reactor and the reactor is maintained in vacuum and then in a N₂ atmosphere. Subsequently, 100 ml of THF was added to the mixture and the resulting mixture was stirred. Then, 13.0 ml (3.0 eq) of triethylamine and 5.2 ml (1.2 eq) of TMS-acetylene were slowly dropped to the stirred mixture and the resultant mixture was then stirred at room temperature for 2 hours in a N₂ atmosphere. Thereafter, a solvent was removed therefrom by using a rotary evaporator and the reaction solution was extracted three times with 100 ml of diethylether and 100 ml of water. The obtained organic layer was dried with magnesium sulfate and a solvent was evaporated therefrom to obtain a crude product. The crude product was purified with silicagel column chromatography to obtain 8.8 g of Intermediate 2-a (yield 84%). The obtained compound was confirmed by LC-MS.

C₂₃H₂₃N₁Si₁: M+341.16

### Synthesis of Intermediate 2-b

8.0 g of Intermediate 2-a was mixed with 100 ml of THF, 60 ml of tetrabutylammonium fluoride (1.0 M in THF) was added to the mixture, and the resulting mixture was then stirred for 30 minutes. Subsequently, 100 ml of water was added to the obtained reaction solution and the reaction solution was extracted three times with 100 ml of ethylether. The obtained organic layer was dried with magnesium sulfate and a solvent was evaporated therefrom to obtain a crude product. The crude product was purified with silicagel column chromatography to obtain 5.6 g of Intermediate 2-b (yield 91%). The obtained compound was confirmed by LC-MS.

C₂₀H₁₅N₁: M+269.12

### Synthesis of Intermediate 2-c

7.1 g (1.2 eq) of 2-bromoiodobenzene, 960 mg (0.04 eq) of Pd(PPh₃)₄, and 320 mg (0.08 eq) of CuI were added to a reactor and the reactor is maintained in vacuum and then in a N₂ atmosphere. Subsequently, 100 ml of THF was added to the mixture and the resulting mixture was stirred. Then, 3.0 ml (1.2 eq) of triethylamine and 5.6 g of Intermediate 2-b were slowly dropped to the stirred mixture and the resultant mixture was then stirred at room temperature for 2 hours in a N₂ atmosphere. Thereafter, a solvent was removed therefrom by using a rotary evaporator, 100 ml of water was added to the reaction solution, and the reaction solution was extracted three times with 100 ml of ethylether. The obtained organic layer was dried with magnesium sulfate and a solvent was evaporated therefrom to obtain a crude product. The crude product was purified with silicagel column chromatography to obtain 5.5 g of Intermediate 2-c (yield 62%). The obtained compound was confirmed by LC-MS.

C₂₆H₁₈Br₁N₁: M+423.06

### Synthesis of Intermediate 2-e

5.0 g of Intermediate 2-c, 5.2 g (1.5 eq) of Intermediate 2-d, 680 mg (0.05 eq) of Pd(PPh₃)₄, and 8.1 g (5.0 eq) of K₂CO₃ were mixed with 100 ml of THF and 30 ml of distilled water, the temperature of the mixed solution was raised to 120° C., and the mixed solution was then stirred by reflux for 24 hours. The obtained reaction solution was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 4.9 g of Intermediate 2-e (yield 82%). The obtained compound was confirmed by LC-MS.

C₃₅H₂₅N₁O₁: M+511.19

### Synthesis of Intermediate 2-f

10.1 g (2.0 eq) of bis(pyridine)iodonium tetrafluoroborate and 80 ml of dichloromethane were mixed together, and 8.3 ml (0.002 eq, d 1.696) of CF₃SO₃H was added to the mixture and the resultant mixture was stirred at −40° C. A mixture of 20 ml of dichloromethane and 4.0 g (1.0 eq) of Intermediate 2-e was added to the reaction solution, the temperature of the reaction solution was raised to 10° C., and the resultant solution was stirred for 2 hours. Thereafter, the temperature of the reaction solution was raised to room temperature and the reaction solution was extracted three times with 100 ml of water and 100 ml of diethylether to obtain an organic layer. The organic layer was dried with magnesium sulfate and a solvent was evaporated therefrom to obtain a crude product. The crude product was purified with silicagel column chromatography to obtain 4.7 g of Intermediate 2-f (yield 95%). The obtained compound was confirmed by LC-MS.

C₃₈H₂₄I₁N₁O₁: M+637.09

### Synthesis of Compound 66

4.0 g of Intermediate 2-f, 2.2 g (1.5 eq) of Intermediate 2-g, 363 mg (0.05 eq) of Pd(PPh₃)₄, and 4.3 g (5.0 eq) of K₂CO₃ were mixed with 100 ml of THF and 30 ml of distilled water, the temperature of the mixed solution was raised to 120° C., and the mixed solution was then stirred by reflux for 24 hours. The obtained reaction solution was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 3.2 g of Compound 66 (yield 76%). The obtained compound was confirmed by LC-MS and NMR.

C₄₉H₃₂N₂O₁: M+664.25

1H NMR (CDCl₃, 400 MHz) δ (ppm) 8.93 (d, 1H), 8.81 (s, 1H), 8.55 (d, 1H), 8.12 (m, 2H), 7.97-7.74 (m, 7H), 7.54-7.13 (m, 13H)

### Synthesis Example 3

### Synthesis of Compound 1

Compound 1 was synthesized in the same manner as in Synthesis Example 1, except that, in the synthesis process of Intermediate 1-a, Intermediate 1-(1) was used instead of 3-iodo-9-phenyl-9H-carbazole. The obtained compound was confirmed by LC-MS and NMR.

C₂₆H₁₆S₁: M+360.10

1H NMR (CDCl₃, 400 MHz) δ (ppm) 8.93 (d, 1H), 8.12 (m, 2H), 7.93-7.78 (m, 6H), 7.48-7.22 (m, 7H)

### Synthesis Example 4

### Synthesis of Compound 2

Compound 2 was synthesized in the same manner as in Synthesis Example 1, except that, in the synthesis process of Intermediate 1-a, Intermediate 1-(2) was used instead of 3-iodo-9-phenyl-9H-carbazole. The obtained compound was confirmed by LC-MS and NMR.

C₂₇H₁₈O₁S₁: M+390.50

1H NMR (CDCl₃, 400 MHz) δ (ppm) 8.93 (d, 1H), 8.12 (m, 2H), 7.93-7.78 (m, 6H), 7.37-7.31 (m, 4H), 6.83 (d, 2H), 3.91 (s, 3H)

### Synthesis Example 5

### Synthesis of Compound 3

Compound 3 was synthesized in the same manner as in Synthesis Example 1, except that, in the synthesis process of Intermediate 1-a, Intermediate 1-(3) was used instead of 3-iodo-9-phenyl-9H-carbazole. The obtained compound was confirmed by LC-MS and NMR.

C₂₄H₁₄S₂: M+366.05

1H NMR (CDCl₃, 400 MHz) δ (ppm) 8.93 (d, 1H), 8.12 (m, 2H), 7.93-7.78 (m, 6H), 7.33-7.20 (m, 4H), 7.01 (d, 1H)

### Synthesis Example 6

### Synthesis of Compound 4

Compound 4 was synthesized in the same manner as in Synthesis Example 1, except that, in the synthesis process of Intermediate 1-a, Intermediate 1-(4) was used instead of 3-iodo-9-phenyl-9H-carbazole. The obtained compound was confirmed by LC-MS and NMR.

C₂₈H₁₆S₂: M+416.07

1H NMR (CDCl₃, 400 MHz) δ (ppm) 8.93 (d, 1H), 8.12 (m, 2H), 7.93-7.78 (m, 8H), 7.4 (s, 1H), 7.33-7.31 (m, 4H)

### Synthesis Example 7

### Synthesis of Compound 5

Compound 5 was synthesized in the same manner as in Synthesis Example 1, except that, in the synthesis process of Intermediate 1-a, Intermediate 1-(5) was used instead of 3-iodo-9-phenyl-9H-carbazole. The obtained compound was confirmed by LC-MS and NMR.

C₂₄H₁₄O₁S₁: M+350.08

1H NMR (CDCl₃, 400 MHz) δ (ppm) 8.93 (d, 1H), 8.12 (m, 2H), 7.93-7.78 (m, 6H), 7.40-7.31 (m, 4H), 6.30 (d, 1H)

### Synthesis Example 8

### Synthesis of Compound 6

Compound 6 was synthesized in the same manner as in Synthesis Example 1, except that, in the synthesis process of Intermediate 1-a, Intermediate 1-(6) was used instead of 3-iodo-9-phenyl-9H-carbazole. The obtained compound was confirmed by LC-MS and NMR.

C₃₄H₂₁N₁S₁: M+475.14

1H NMR (CDCl₃, 400 MHz) δ (ppm) 8.93 (d, 1H), 8.12 (m, 2H), 7.93-7.78 (m, 6H), 7.36-7.17 (m, 12H)

### Synthesis Example 9

### Synthesis of Compound 8

Compound 8 was synthesized in the same manner as in Synthesis Example 1, except that, in the synthesis process of Intermediate 1-a, Intermediate 1-(8) was used instead of 3-iodo-9-phenyl-9H-carbazole. The obtained compound was confirmed by LC-MS and NMR.

C₃₅H₂₄S₁: M+476.16

1H NMR (CDCl₃, 400 MHz) δ (ppm) 8.93 (d, 1H), 8.12 (m, 2H), 7.93-7.77 (m, 9H), 7.60-7.55 (m, 2H), 7.38-7.28 (m, 4H), 1.67 (s, 6H)

### Synthesis Example 10

### Synthesis of Compound 9

Compound 9 was synthesized in the same manner as in Synthesis Example 1, except that, in the synthesis process of Intermediate 1-a, Intermediate 1-(9) was used instead of 3-iodo-9-phenyl-9H-carbazole. The obtained compound was confirmed by LC-MS and NMR.

C₃₈H₂₃N₁S₁: M+525.16

1H NMR (CDCl₃, 400 MHz) δ (ppm) 8.93 (d, 1H), 8.12 (m, 2H), 7.93-7.78 (m, 6H), 7.55-7.30 (m, 10H), 7.08-7.00 (m, 4H)

### Synthesis Example 11

### Synthesis of Compound 10

Compound 10 was synthesized in the same manner as in Synthesis Example 1, except that, in the synthesis process of Intermediate 1-a, (4-bromo-phenyl)-diphenyl-amine was used instead of 3-iodo-9-phenyl-9H-carbazole. The obtained compound was confirmed by LC-MS and NMR.

C₃₈H₂₅N₁S₁: M+527.17

1H NMR (CDCl₃, 400 MHz) δ (ppm) 8.93 (d, 1H), 8.12 (m, 2H), 7.93-7.78 (m, 6H), 7.33-7.01 (m, 8H), 6.62-6.46 (m, 8H)

### Synthesis Example 12

### Synthesis of Compound 11

Compound 11 was synthesized in the same manner as in Synthesis Example 1, except that, in the synthesis process of Intermediate 1-a, Intermediate 1-(11) was used instead of 3-iodo-9-phenyl-9H-carbazole. The obtained compound was confirmed by LC-MS and NMR.

C₃₂H₁₈S₂: M+455.08

1H NMR (CDCl₃, 400 MHz) δ (ppm) 8.93 (d, 1H), 8.12 (m, 2H), 7.93-7.74 (m, 9H), 7.53 (d, 1H), 7.39-7.31 (m, 5H)

### Synthesis Example 13

### Synthesis of Compound 12

Compound 12 was synthesized in the same manner as in Synthesis Example 1, except that, in the synthesis process of Intermediate 1-a, Intermediate 1-(12) was used instead of 3-iodo-9-phenyl-9H-carbazole. The obtained compound was confirmed by LC-MS and NMR.

C₃₂H₁₈O₂: M+450.11

1H NMR (CDCl₃, 400 MHz) δ (ppm) 8.93 (d, 1H), 8.12 (m, 2H), 7.93-7.78 (m, 6H), 7.49-7.13 (m, 9H)

### Synthesis Example 14

### Synthesis of Compound 13

Compound 13 was synthesized in the same manner as in Synthesis Example 1, except that, in the synthesis process of Intermediate 1-a, Intermediate 1-(13) was used instead of 3-iodo-9-phenyl-9H-carbazole. The obtained compound was confirmed by LC-MS and NMR.

C₃₅H₂₁N₃S₁: M+515.15

1H NMR (CDCl₃, 400 MHz) δ (ppm) 8.93 (d, 1H), 8.12 (m, 2H), 7.93-7.78 (m, 6H), 7.48-7.22 (m, 12H)

### Synthesis Example 15

### Synthesis of Compound 14

Compound 14 was synthesized in the same manner as in Synthesis Example 2, except that Intermediate 1-(2) was used instead of (4-bromo-phenyl)-diphenyl-amine in the synthesis process of Intermediate 2-a, Intermediate 1-d was used instead of Intermediate 2-d in the synthesis process of Intermediate 2-e, and Intermediate 4-(14) was used instead of Intermediate 2-g in the synthesis process of Compound 66. The obtained compound was confirmed by LC-MS and NMR.

C₄H₃₀O₂S₂: M+702.17

1H NMR (CDCl₃, 400 MHz) δ (ppm) 8.93 (d, 1H), 8.12 (m, 2H), 7.88-7.78 (m, 5H), 7.48-7.22 (m, 17H), 6.83 (d, 2H), 3.73 (s, 3H)

### Synthesis Example 16

### Synthesis of Compound 15

Compound 15 was synthesized in the same manner as in Synthesis Example 2, except that Intermediate 1-(2) was used instead of (4-bromo-phenyl)-diphenyl-amine in the synthesis process of Intermediate 2-a, Intermediate 3-(15) was used instead of Intermediate 2-d in the synthesis process of Intermediate 2-e, and Intermediate 4-(15) was used instead of Intermediate 2-g in the synthesis process of Compound 66. The obtained compound was confirmed by LC-MS and NMR.

C₃₆H₂₃N₁O₁S₁: M+517.15

1H NMR (CDCl₃, 400 MHz) δ (ppm) 8.93 (d, 1H), 8.81 (s, 1H), 8.55 (d, 1H), 8.12-7.67 (m, 9H), 7.44-7.31 (m, 6H), 6.83 (d, 2H) 3.73 (s, 3H)

### Synthesis Example 17

### Synthesis of Compound 16

Compound 16 was synthesized in the same manner as in Synthesis Example 2, except that Intermediate 1-(2) was used instead of (4-bromo-phenyl)-diphenyl-amine in the synthesis process of Intermediate 2-a, Intermediate 3-(16) was used instead of Intermediate 2-d in the synthesis process of Intermediate 2-e, and intermediate 4-(16) was used instead of Intermediate 2-g in the synthesis process of Compound 66. The obtained compound was confirmed by LC-MS and NMR.

C₄₁H₂₆O₁S₁: M+566.17

1H NMR (CDCl₃, 400 MHz) δ (ppm) 8.93 (d, 1H), 8.12 (m, 2H), 7.89-7.54 (m, 14H), 7.37-7.32 (m, 6H), 3.73 (s, 3H)

### Synthesis Example 18

### Synthesis of Compound 17

Compound 17 was synthesized in the same manner as in Synthesis Example 1, except that, in the synthesis process of Intermediate 1-a, Intermediate 1-(17) was used instead of 3-iodo-9-phenyl-9H-carbazole. The obtained compound was confirmed by LC-MS and NMR.

C₃₄H₂₀S₁: M+460.13

1H NMR (CDCl₃, 400 MHz) δ (ppm) 8.93 (d, 3H), 8.12 (m, 4H), 7.93-7.78 (m, 11H), 7.33-7.31 (m, 2H)

### Synthesis Example 19

### Synthesis of Compound 18

Compound 18 was synthesized in the same manner as in Synthesis Example 1, except that, in the synthesis process of Intermediate 1-a, Intermediate 1-(18) was used instead of 3-iodo-9-phenyl-9H-carbazole. The obtained compound was confirmed by LC-MS and NMR.

C₃₆H₂₀S₁: M+484.13

1H NMR (CDCl₃, 400 MHz) δ (ppm) 8.93 (d, 1H), 8.18-7.71 (m, 17H), 7.33-7.31 (m, 2H)

### Synthesis Example 20

### Synthesis of Compound 19

Compound 19 was synthesized in the same manner as in Synthesis Example 1, except that, in the synthesis process of Intermediate 1-a, Intermediate 1-(19) was used instead of 3-iodo-9-phenyl-9H-carbazole. The obtained compound was confirmed by LC-MS and NMR.

C₃₀H₁₈S₁: M+410.11

1H NMR (CDCl₃, 400 MHz) δ (ppm) 8.93 (d, 2H), 8.12 (m, 4H), 7.93-7.13 (m, 12H)

### Synthesis Example 21

### Synthesis of Compound 20

Compound 20 was synthesized in the same manner as in Synthesis Example 1, except that Intermediate 1-(20) was used instead of 3-iodo-9-phenyl-9H-carbazole in the synthesis process of Intermediate 1-a and Intermediate 3-(20) was used instead of Intermediate 1-d in the synthesis process of Intermediate 1-e. The obtained compound was confirmed by LC-MS and NMR.

C₃₆H₂₂S₁: M+486.14

1H NMR (CDCl₃, 400 MHz) δ (ppm) 8.93 (d, 1H), 8.12 (m, 2H), 7.93-7.22 (m, 19H)

### Synthesis Example 22

### Synthesis of Compound 21

Compound 21 was synthesized in the same manner as in Synthesis Example 1, except that, in the synthesis process of Intermediate 1-a, Intermediate 1-(21) was used instead of 3-iodo-9-phenyl-9H-carbazole. The obtained compound was confirmed by LC-MS and NMR.

C₃₁H₂₀O₁S₁: M+440.12

1H NMR (CDCl₃, 400 MHz) δ (ppm) 8.93 (d, 1H), 8.17-8.12 (m, 3H), 7.93-7.31 (m, 12H), 6.70 (d, 1H), 3.73 (s, 3H)

### Synthesis Example 23

### Synthesis of Compound 22

Compound 22 was synthesized in the same manner as in Synthesis Example 1, except that, in the synthesis process of Intermediate 1-a, Intermediate 1-(22) was used instead of 3-iodo-9-phenyl-9H-carbazole. The obtained compound was confirmed by LC-MS and NMR.

C₃₈H₂₈S₁: M+516.19

1H NMR (CDCl₃, 400 MHz) δ (ppm) 8.93 (d, 1H), 8.12 (m, 2H), 7.96-7.78 (m, 10H), 7.38-7.31 (6H), 1.47 (s, 9H)

### Synthesis Example 24

### Synthesis of Compound 23

Compound 23 was synthesized in the same manner as in Synthesis Example 1, except that Intermediate 1-(17) was used instead of 3-iodo-9-phenyl-9H-carbazole in the synthesis process of Intermediate 1-a and Intermediate 2-(23) was used instead of 2-bromoiodobenzene in the synthesis process of Intermediate 1-c. The obtained compound was confirmed by LC-MS and NMR.

C₃₅H₁₉N₁S₁: M+485.12

1H NMR (CDCl₃, 400 MHz) δ (ppm) 9.18 (s, 1H), 8.93 (d, 2H), 8.31-7.78 (m, 14H), 7.33-7.31 (m, 2H)

### Synthesis Example 25

### Synthesis of Compound 24

Compound 24 was synthesized in the same manner as in Synthesis Example 1, except that Intermediate 1-(17) was used instead of 3-iodo-9-phenyl-9H-carbazole in the synthesis process of Intermediate 1-a and Intermediate 2-(24) was used instead of 2-bromoiodobenzene in the synthesis process of Intermediate 1-c. The obtained compound was confirmed by LC-MS and NMR.

C₄₀H₂₄S₁: M+526.16

1H NMR (CDCl₃, 400 MHz) δ (ppm) 8.93-8.89 (m, 3H), 8.12-7.78 (m, 14H), 7.48-7.22 (m, 7H)

### Synthesis Example 26

### Synthesis of Compound 25

Compound 25 was synthesized in the same manner as in Synthesis Example 1, except that Intermediate 1-(17) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₄H₂₀O₁: M+444.15

1H NMR (CDCl₃, 400 MHz) δ (ppm) 8.93 (d, 3H), 8.12 (m, 4H), 7.93-7.82 (m, 9H), 7.49-7.42 (m, 2H), 7.19-7.13 (m, 2H)

### Synthesis Example 27

### Synthesis of Compound 26

Compound 26 was synthesized in the same manner as in Synthesis Example 2, except that Intermediate 1-(20) was used instead of (4-bromo-phenyl)-diphenyl-amine in the synthesis process of Intermediate 2-a, Intermediate 2-(26) was used instead of 2-bromoiodobenzene in the synthesis process of Intermediate 2-c, and Intermediate 4-(26) was used instead of Intermediate 2-g in the synthesis process of Compound 66. The obtained compound was confirmed by LC-MS and NMR.

C₄₂H₂₆O₁S₁: M+578.17

1H NMR (CDCl₃, 400 MHz) δ (ppm) 9.15 (s, 1H), 8.18-8.04 (m, 3H), 7.82 (d, 1H), 7.54-7.00 (m, 21H)

### Synthesis Example 28

### Synthesis of Compound 27

Compound 27 was synthesized in the same manner as in Synthesis Example 1, except that Intermediate 1-(19) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₀H₁₈O₁: M+394.14

1H NMR (CDCl₃, 400 MHz) δ (ppm) 8.93 (d, 1H), 8.12 (m, 2H), 7.93-7.13 (m, 15H)

### Synthesis Example 29

### Synthesis of Compound 28

Compound 28 was synthesized in the same manner as in Synthesis Example 1, except that Intermediate 1-(19) was used instead of 3-iodo-9-phenyl-9H-carbazole in the synthesis process of Intermediate 1-a, Intermediate 2-(28) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₃₆H₁₀F₂O₁: M+506.15

1H NMR (CDCl₃, 400 MHz) δ (ppm) 8.99 (d, 1H), 8.34 (s, 1H), 8.12-8.10 (m, 2H), 7.93 (s, 1H), 7.82 (d, 1H), 7.67-6.91 (m, 14H)

### Synthesis Example 30

### Synthesis of Compound 29

Compound 29 was synthesized in the same manner as in Synthesis Example 1, except that Intermediate 1-(21) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃H₂₀O₂: M+424.15

1H NMR (CDCl₃, 400 MHz) δ (ppm) 8.93 (d, 1H), 8.17-8.12 (m, 3H), 7.93-7.13 (m, 12H), 6.70 (d, 1H), 3.73 (s, 3H)

### Synthesis Example 31

### Synthesis of Compound 30

Compound 30 was synthesized in the same manner as in Synthesis Example 1, except that Intermediate 1-(30) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₄₀H₂₄O₁: M+520.18

1H NMR (CDCl₃, 400 MHz) δ (ppm) 8.93 (d, 1H), 8.12 (m, 2H), 7.91-7.82 (m, 8H), 7.49-7.13 (m, 13H)

### Synthesis Example 32

### Synthesis of Compound 31

Compound 31 was synthesized in the same manner as in Synthesis Example 2, except that Intermediate 1-(17) was used instead of (4-bromo-phenyl)-diphenyl-amine in the synthesis process of Intermediate 2-a, Intermediate 3-(31) was used instead of Intermediate 2-d in the synthesis process of Intermediate 2-e, and Intermediate 4-(15) was used instead of Intermediate 2-g in the synthesis process of Compound 66. The obtained compound was confirmed by LC-MS and NMR.

C₄₃H₂₅N₁O₁: M+571.19

1H NMR (CDCl₃, 400 MHz) δ (ppm) 8.93 (m, 3H), 8.81 (s, 1H), 8.55 (d, 1H), 8.12-7.67 (m, 15H), 7.42-7.19 (m, 5H)

### Synthesis Example 33

### Synthesis of Compound 32

Compound 32 was synthesized in the same manner as in Synthesis Example 2, except that Intermediate 1-(17) was used instead of (4-bromo-phenyl)-diphenyl-amine in the synthesis process of Intermediate 2-a, Intermediate 2-(26) was used instead of 2-bromoiodobenzene in the synthesis process of Intermediate 2-c, and Intermediate 4-(15) was used instead of Intermediate 2-g in the synthesis process of Compound 66. The obtained compound was confirmed by LC-MS and NMR.

C₄₅H₂₇N₁O₁: M+597.21

1H NMR (CDCl₃, 400 MHz) δ (ppm) 9.15 (s, 1H), 8.93 (d, 2H), 8.81 (s, 1H), 8.55 (d, 1H), 8.18-7.82 (m, 12H), 7.49-7.13 (m, 10H)

### Synthesis Example 34

### Synthesis of Compound 33

Compound 33 was synthesized in the same manner as in Synthesis Example 2, except that Intermediate 1-(4) was used instead of (4-bromo-phenyl)-diphenyl-amine in the synthesis process of Intermediate 2-a, Intermediate 1-d was used instead of Intermediate 2-d in the synthesis process of Intermediate 2-e, and Intermediate 4-(33) was used instead of Intermediate 2-g in the synthesis process of Compound 66. The obtained compound was confirmed by LC-MS and NMR.

C₄₃H₂₈S₂: M+608.16

1H NMR (CDCl₃, 400 MHz) δ (ppm) 8.93 (d, 1H), 8.12 (m, 2H), 7.90-7.28 (m, 19H), 1.67 (s, 6H)

### Synthesis Example 35

### Synthesis of Compound 34

Compound 34 was synthesized in the same manner as in Synthesis Example 2, except that Intermediate 1-(4) was used instead of (4-bromo-phenyl)-diphenyl-amine in the synthesis process of Intermediate 2-a, Intermediate 1-d was used instead of Intermediate 2-d in the synthesis process of Intermediate 2-e, and Intermediate 4-(34) was used instead of Intermediate 2-g in the synthesis process of Compound 66. The obtained compound was confirmed by LC-MS and NMR.

C₄₆H₂₇N₁S₂: M+657.17

1H NMR (CDCl₃, 400 MHz) δ (ppm) 8.93 (d, 1H), 8.12 (m, 2H), 7.90-7.77 (m, 8H), 7.55-7.30 (m, 14H), 7.08-7.00 (m, 2H)

### Synthesis Example 36

### Synthesis of Compound 35

Compound 35 was synthesized in the same manner as in Synthesis Example 2, except that Intermediate 1-d was used instead of Intermediate 2-d in the synthesis process of Intermediate 2-e and Intermediate 4-(15) was used instead of Intermediate 2-g in the synthesis process of Compound 66. The obtained compound was confirmed by LC-MS and NMR.

C₄₃H₂₈N₂S₁: M+640.20

1H NMR (CDCl₃, 400 MHz) δ (ppm) 8.93 (d, 1H), 8.65 (d, 2H), 8.12 (d, 2H), 7.88-7.78 (m, 5H), 7360 (d, 2H), 7.33-7.23 (m, 4H), 7.01 (m, 4H), 6.62-6.46 (m, 8H)

### Synthesis Example 37

### Synthesis of Compound 36

Compound 36 was synthesized in the same manner as in Synthesis Example 2, except that Intermediate 1-(11) was used instead of (4-bromo-phenyl)-diphenyl-amine in the synthesis process of Intermediate 2-a and Intermediate 1-d was used instead of Intermediate 2-d in the synthesis process of Intermediate 2-e. The obtained compound was confirmed by LC-MS and NMR.

C₄₃H₂₅N₁S₂: M+619.14

1H NMR (CDCl₃, 400 MHz) δ (ppm) 8.93 (m, 1H), 8.81 (s, 1H), 8.55 (d, 1H), 8.12-7.31 (m, 22H)

### Synthesis Example 38

### Synthesis of Compound 37

Compound 37 was synthesized in the same manner as in Synthesis Example 2, except that Intermediate 1-(11) was used instead of (4-bromo-phenyl)-diphenyl-amine in the synthesis process of Intermediate 2-a, Intermediate 3-(16) was used instead of Intermediate 2-d and Intermediate 4-(37) was used instead of Intermediate 2-g in the synthesis process of Intermediate 2-e. The obtained compound was confirmed by LC-MS and NMR.

C₅₂H₂₈S₂: M+716.16

1H NMR (CDCl₃, 400 MHz) δ (ppm) 8.93 (m, 1H), 8.12-7.32 (m, 27H)

### Synthesis Example 39

### Synthesis of Compound 38

Compound 38 was synthesized in the same manner as in Synthesis Example 2, except that Intermediate 1-(12) was used instead of (4-bromo-phenyl)-diphenyl-amine in the synthesis process of Intermediate 2-a, Intermediate 1-d was used instead of Intermediate 2-d and Intermediate 4-(38) was used instead of Intermediate 2-g in the synthesis process of Intermediate 2-e. The obtained compound was confirmed by LC-MS and NMR.

C₃₈H₂₀F₂O₁S₁: M+562.12

1H NMR (CDCl₃, 400 MHz) δ (ppm) 8.93 (d, 1H), 8.12 (m, 2H), 7.88-7.78 (m, 5H), 7.49-7.01 (m, 12H)

### Synthesis Example 40

### Synthesis of Compound 39

Compound 39 was synthesized in the same manner as in Synthesis Example 2, except that Intermediate 1-(13) was used instead of (4-bromo-phenyl)-diphenyl-amine in the synthesis process of Intermediate 2-a, Intermediate 1-d was used instead of Intermediate 2-d and Intermediate 4-(39) was used instead of Intermediate 2-g in the synthesis process of Intermediate 2-e. The obtained compound was confirmed by LC-MS and NMR.

C₄₄H₂₆N₄S₁: M+642.19

1H NMR (CDCl₃, 400 MHz) δ (ppm) 8.93 (d, 1H), 8.42 (d, 1H), 8.12 (m, 2H), 88-7.10 (m, 22H)

### Synthesis Example 41

### Synthesis of Compound 40

Compound 40 was synthesized in the same manner as in Synthesis Example 2, except that Intermediate 1-(12) was used instead of (4-bromo-phenyl)-diphenyl-amine in the synthesis process of Intermediate 2-a, Intermediate 1-d was used instead of Intermediate 2-d and Intermediate 4-(40) was used instead of Intermediate 2-g in the synthesis process of Intermediate 2-e. The obtained compound was confirmed by LC-MS and NMR.

C₃₉H₂₁N₁O₁S₁: M+551.13

1H NMR (CDCl₃, 400 MHz) δ (ppm) 8.93 (d, 1H), 8.12 (m, 2H), 7.88-7.13 (m, 18H),

### Synthesis Example 42

### Synthesis of Compound 41

Compound 41 was synthesized in the same manner as in Synthesis Example 1, except that Intermediate 1-(2) was used instead of 3-iodo-9-phenyl-9H-carbazole in the synthesis process of Intermediate 1-a and Intermediate 2-(41) was used instead of 2-bromoiodobenzene in the synthesis process of Intermediate 1-c. The obtained compound was confirmed by LC-MS and NMR.

C₃₂H₂₂O₁S₁: M+466.14

1H NMR (CDCl₃, 400 MHz) δ (ppm) 8.99 (d, 1H), 8.34 (S, 1H), 8.12-8.10 (m, 2H), 7.93-7.78 (m, 4H), 7.48-7.22 (m, 9H), 6.83 (d, 2H), 3.73 (s, 3H)

### Synthesis Example 43

### Synthesis of Compound 42

Compound 42 was synthesized in the same manner as in Synthesis Example 1, except that (4-bromo-phenyl)-diphenyl-amine was used instead of 3-iodo-9-phenyl-9H-carbazole in the synthesis process of Intermediate 1-a and Intermediate 2442) was used instead of 2-bromoiodobenzene in the synthesis process of Intermediate 1-c. The obtained compound was confirmed by LC-MS and NMR.

C₄₂H₃₃N₁S₁: M+583.23

1H NMR (CDCl₃, 400 MHz) δ (ppm) 8.85 (d, 1H), 8.15-8.12 (m, 2H), 7.93-7.78 (m, 5H), 7.32-7.23 (m, 4H), 7.01 (m, 4H), 6.62-6.46 (m, 8H), 1.40 (m, 9H)

### Synthesis Example 44

### Synthesis of Compound 43

Compound 43 was synthesized in the same manner as in Synthesis Example 1, except that (4-bromo-phenyl)-diphenyl-amine was used instead of 3-iodo-9-phenyl-9H-carbazole in the synthesis process of Intermediate 1-a, Intermediate 2-(23) was used instead of 2-bromoiodobenzene in the synthesis process of Intermediate 1-c, and Intermediate 3-(20) was used instead of Intermediate 1-d in the synthesis process of Intermediate 1-e. The obtained compound was confirmed by LC-MS and NMR.

C₄₃H₂₆N₂S₁: M+602.18

1H NMR (CDCl₃, 400 MHz) δ (ppm) 9.18 (d, 1H), 8.30-7.67 (m, 8H), 7.33-7.01 (m, 9H), 6.62-6.46 (m, 8H)

### Synthesis Example 45

### Synthesis of Compound 44

Compound 44 was synthesized in the same manner as in Synthesis Example 1, except that Intermediate 1-(2) was used instead of 3-iodo-9-phenyl-9H-carbazole in the synthesis process of Intermediate 1-a and Intermediate 2-(24) was used instead of 2-bromoiodobenzene in the synthesis process of Intermediate 1-c. The obtained compound was confirmed by LC-MS and NMR.

C₃₂H₂₁N₁O₁S₁: M+467.13

1H NMR (CDCl₃, 400 MHz) δ (ppm) 8.81 (s, 1H), 8.55 (d, 1H), 8.12-7.78 (m, 9H), 7.44-7.31 (m, 5H), 6.83 (d, 2H), 3.73 (s, 3H)

### Synthesis Example 46

### Synthesis of Compound 45

Compound 45 was synthesized in the same manner as in Synthesis Example 1, except that (4-bromo-phenyl)-diphenyl-amine was used instead of 3-iodo-9-phenyl-9H-carbazole in the synthesis process of Intermediate 1-a and Intermediate 2-(45) was used instead of 2-bromoiodobenzene in the synthesis process of Intermediate 1-c. The obtained compound was confirmed by LC-MS and NMR.

C₄₆H₂₉N₁O₁S₁: M+643.20

1H NMR (CDCl₃, 400 MHz) δ (ppm) 8.89 (d, 1H), 8.12-7.78 (m, 7H), 7.50-7.01 (m, 13H), 6.62-6.46 (m, 8H)

### Synthesis Example 47

### Synthesis of Compound 46

Compound 46 was synthesized in the same manner as in Synthesis Example 1, except that Intermediate 1-(2) was used instead of 3-iodo-9-phenyl-9H-carbazole in the synthesis process of Intermediate 1-a and Intermediate 2-(46) was used instead of 2-bromoiodobenzene in the synthesis process of Intermediate 1-c. The obtained compound was confirmed by LC-MS and NMR.

C₃₇H₂₄O₁S₂: M+548.13

1H NMR (CDCl₃, 400 MHz) δ (ppm) 9.21 (s, 1H), 8.40 (s, 1H), 8.12 (d, 1H), 7.93-7.78 (m, 4H), 7.48-7.20 (m, 10H), 7.00-6.83 (m, 4H), 3.73 (s, 3H)

### Synthesis Example 48

### Synthesis of Compound 47

Compound 47 was synthesized in the same manner as in Synthesis Example 1, except that Intermediate 1-(1) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₂₆H₁₆O₁: M+344.12

1H NMR (CDCl₃, 400 MHz) δ (ppm) 8.93 (d, 1H), 8.12 (m, 2H), 7.93-7.82 (m, 4H), 7.49-7.13 (m, 9H)

### Synthesis Example 49

### Synthesis of Compound 48

Compound 48 was synthesized in the same manner as in Synthesis Example 1, except that Intermediate 1-(2) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₂₇H₁₈O₂: M+374.13

1H NMR (CDCl₃, 400 MHz) δ (ppm) 8.93 (d, 1′-1), 8.12 (m, 2H), 7.93-7.82 (m, 4H), 7.49-7.13 (m, 8H), 6.83 (d, 2H), 3.73 (s, 3H)

### Synthesis Example 50

### Synthesis of Compound 49

Compound 49 was synthesized in the same manner as in Synthesis Example 1, except that Intermediate 1-(3) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₂₄H₁₄O₁S₁: M+350.08

1H NMR(CDCl₃, 400 MHz) δ (ppm) 8.93 (d, 1H), 8.12 (m, 2H), 7.93-7.82 (m, 4H), 7.49-7.00 (m, 7H)

### Synthesis Example 51

### Synthesis of Compound 50

Compound 50 was synthesized in the same manner as in Synthesis Example 1, except that Intermediate 1-(4) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₂₃H₁₆O₁S₁: M+400.09

1H NMR (CDCl₃, 400 MHz) δ (ppm) 8.93 (d, 1H), 8.12 (m, 2H), 7.93-7.80 (m, 6H), 7.49-7.13 (m, 7H)

### Synthesis Example 52

### Synthesis of Compound 51

Compound 51 was synthesized in the same manner as in Synthesis Example 1, except that Intermediate 1-(5) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₂₄H₁₄O₂: M+334.10

1H NMR (CDCl₃, 400 MHz) δ (ppm) 8.93 (d, 1H), 8.12 (m, 2H), 7.93-7.82 (m, 4H), 7.49-7.13 (m, 6H), 6.30 (d, 1H)

### Synthesis Example 53

### Synthesis of Compound 52

Compound 52 was synthesized in the same manner as in Synthesis Example 1, except that Intermediate 1-(6) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₄H₂₁N₁O₁: M+459.16

1H NMR (CDCl₃, 400 MHz) δ (ppm) 8.93 (d, 1H), 8.12 (m, 2H), 7.93-7.82 (m, 4H), 7.49-7.13 (m, 14H)

### Synthesis Example 54

### Synthesis of Compound 53

Compound 53 was synthesized in the same manner as in Synthesis Example 1, except that Intermediate 3-(3 I) was used instead of Intermediate 1-d in the synthesis process of Intermediate 1-e. The obtained compound was confirmed by LC-MS and NMR.

C₄₂H₂₅N₁O₁: M+559.19

1H NMR (CDCl₃, 400 MHz) δ (ppm) 8.93 (d, 1H), 8.12 (m, 2H), 7.93-7.00 (m, 22H)

### Synthesis Example 55

### Synthesis of Compound 54

Compound 54 was synthesized in the same manner as in Synthesis Example 1, except that Intermediate 1-(8) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₅H₂₄O₁: M+460.18

1H NMR (CDCl₃, 400 MHz) δ (ppm) 8.93 (d, 1H), 8.12 (m, 2H), 7.93-7.13 (m, 15H), 1.67 (s, 6H)

### Synthesis Example 56

### Synthesis of Compound 55

Compound 55 was synthesized in the same manner as in Synthesis Example 1, except that Intermediate 1-(9) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₈H₂₃N₁O₁: M+509.18

1H NMR (CDCl₃, 400 MHz) δ (ppm) 8.93 (d, 1H), 8.12 (m, 2H), 7.93-7.00 (m, 20H)

### Synthesis Example 57

### Synthesis of Compound 56

Compound 56 was synthesized in the same manner as in Synthesis Example 1, except that (4-bromo-phenyl)-diphenyl-amine was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₈H₂₅N₁O₁: M+511.19

1H NMR (CDCl₃, 400 MHz) δ (ppm). 8.93 (d, 1H), 8.12 (m, 2H), 7.93-7.82 (m, 4H), 7.49-7.01 (m, 10H), 6.62-6.46 (m, 8H)

### Synthesis Example 58

### Synthesis of Compound 57

Compound 57 was synthesized in the same manner as in Synthesis Example 1, except that Intermediate 1-(11) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₂H₁₈O₁S₁: M+450.11

1H NMR (CDCl₃, 400 MHz) δ (ppm) 8.93 (d, 1H), 8.12 (m, 2H), 7.93-7.74 (m, 7H), 7.53-7.13 (m, 8H)

### Synthesis Example 59

### Synthesis of Compound 58

Compound 58 was synthesized in the same manner as in Synthesis Example 1, except that Intermediate 1-(12) was used instead of 3-iodo-9-phenyl-9H-carbazole in the synthesis process of Intermediate 1-a and Intermediate 2-d was used instead of Intermediate 1-d in the synthesis process of Intermediate 1-e. The obtained compound was confirmed by LC-MS and NMR.

C₃₂H₁₃O₂: M+434.13

1H NMR (CDCl₃, 400 MHz) δ (ppm) 8.93 (d, 1H), 8.12 (m, 2H), 7.93-7.82 (m, 4H), 7.49-7.41 (m, 6H), 7.19-7.13 (m, 5H)

### Synthesis Example 60

### Synthesis of Compound 59

Compound 59 was synthesized in the same manner as in Synthesis Example 1, except that Intermediate 1-(13) was used instead of 3-iodo-9-phenyl-9H-carbazole in the synthesis process of Intermediate 1-a and Intermediate 3-(59) was used instead of Intermediate 1-d in the synthesis process of Intermediate 1-e. The obtained compound was confirmed by LC-MS and NMR.

C₃₉H₂₃N₃P₁: M+549.18

1H NMR (CDCl₃, 400 MHz) δ (ppm) 8.93 (d, 1H), 8.12 (m, 2H), 7.93-7.67 (m, 6H), 7.49-7.22 (m, 14H)

### Synthesis Example 61

### Synthesis of Compound 60

Compound 60 was synthesized in the same manner as in Synthesis Example 2, except that Intermediate 1-(2) was used instead of (4-bromo-phenyl)-diphenyl-amine in the synthesis process of Intermediate 2-a and Intermediate 4-(14) was used instead of Intermediate 2-g in the synthesis process of Compound 66. The obtained compound was confirmed by LC-MS and NMR.

C₃₃H₂₂O₂: M+450.16

1H NMR (CDCl₃, 400 MHz) δ (ppm) 8.93 (d, 1H), 8.12 (m, 2H), 7.88-7.82 (m, 3H), 7.49-7.13 (m, 1H), 6.83 (d, 2H), 3.73 (s, 3H)

### Synthesis Example 62

### Synthesis of Compound 61

Compound 61 was synthesized in the same manner as in Synthesis Example 2, except that Intermediate 1-(2) was used instead of (4-bromo-phenyl)-diphenyl-amine in the synthesis process of Intermediate 2-a and Intermediate 4-(15) was used instead of Intermediate 2-g in the synthesis process of Compound 66. The obtained compound was confirmed by LC-MS and NMR.

C₃₂H₂N₁O₂: M+451.16

1H NMR (CDCl₃, 400 MHz) δ (ppm) 8.93 (d, 1H), 8.81 (s, 1H), 8.55 (d, 1H), 8.12-7.82 (m, 6H), 7.49-7.13 (m, 9H), 3.73 (s, 3H)

### Synthesis Example 63

### Synthesis of Compound 62

Compound 62 was synthesized in the same manner as in Synthesis Example 2, except that Intermediate 1-(2) was used instead of (4-bromo-phenyl)-diphenyl-amine in the synthesis process of Intermediate 2-a and Intermediate 4-(I 6) was used instead of Intermediate 2-g in the synthesis process of Compound 66. The obtained compound was confirmed by LC-MS and NMR.

C₃₇H₂₄O₂: M+500.18

1H NMR (CDCl₃, 400 MHz) δ (ppm) 8.93 (d, 1H), 8.12 (m, 2H), 7.89-7.13 (m, 16H), 6.83 (d, 2H), 3.73 (s, 3H)

### Synthesis Example 64

### Synthesis of Compound 63

Compound 63 was synthesized in the same manner as in Synthesis Example 2, except that Intermediate 1-(4) was used instead of (4-bromo-phenyl)-diphenyl-amine in the synthesis process of Intermediate 2-a and Intermediate 4-(33) was used instead of Intermediate 2-g in the synthesis process of Compound 66. The obtained compound was confirmed by LC-MS and NMR.

C₄₃H₂₈O₁S₁: M+592.19

1H NMR (CDCl₃, 400 MHz) δ (ppm) 8.93 (d, 1H), 8.12 (m, 2H), 7.90-7.13 (m, 19H), 1.67 (s, 6H)

### Synthesis Example 65

### Synthesis of Compound 64

Compound 64 was synthesized in the same manner as in Synthesis Example 2, except that Intermediate 1-(4) was used instead of (4-bromo-phenyl)-diphenyl-amine in the synthesis process of Intermediate 2-a and Intermediate 4-(34) was used instead of Intermediate 2-g in the synthesis process of Compound 66. The obtained compound was confirmed by LC-MS and NMR.

C₄₆H₂₇N₁O₁S₁: M+641.18

1H NMR (CDCl₃, 400 MHz) δ (ppm) 8.93 (d, 1H), 8.12 (m, 2H), 7.90-7.77 (m, 6H), 7.55-7.00 (m, 18H)

### Synthesis Example 66

### Synthesis of Compound 65

Compound 65 was synthesized in the same manner as in Synthesis Example 2, except that Intermediate 4-(15) was used instead of Intermediate 2-g in the synthesis process of Compound 66. The obtained compound was confirmed by LC-MS and NMR.

C₄₃H₂₈N₂O₁: M+588.22

1H NMR (CDCl₃, 400 MHz) δ (ppm) 8.93 (d, 1H), 8.65 (d, 2H), 8.12 (m, 2H), 7.88-7.82 (m, 3H), 7.60-7.42 (m, 4H), 7.23-7.01 (m, 8H), 6.62-6.46 (m, 8H)

### Synthesis Example 67

### Synthesis of Compound 67

Compound 67 was synthesized in the same manner as in Synthesis Example 2, except that Intermediate 1-(11) was used instead of (4-bromo-phenyl)-diphenyl-amine in the synthesis process of Intermediate 2-a and Intermediate 4-(37) was used instead of Intermediate 2-g in the synthesis process of Compound 66. The obtained compound was confirmed by LC-MS and NMR.

C₄₈H₂₆O₁S₁: M+650.17

1H NMR (CDCl₃, 400 MHz) δ (ppm) 8.93 (d, 1H), 8.12-7.13 (m, 25H)

### Synthesis Example 68

### Synthesis of Compound 68

Compound 68 was synthesized in the same manner as in Synthesis Example 2, except that Intermediate 1-(12) was used instead of (4-bromo-phenyl)-diphenyl-amine in the synthesis process of Intermediate 2-a, Intermediate 3-(31) was used instead of Intermediate 2-d in the synthesis process of Intermediate 2-e, and Intermediate 4-(38) was used instead of Intermediate 2-g in the synthesis process of Compound 66. The obtained compound was confirmed by LC-MS and NMR.

C₄₂H₂₂F₂O₂: M+596.16

1H NMR (CDCl₃, 400 MHz) δ (ppm) 8.93 (d, 1H), 8.12 (m, 2H), 7.88-7.67 (m, 5H), 7.49-7.01 (m, 14H)

### Synthesis Example 69

### Synthesis of Compound 69

Compound 69 was synthesized in the same manner as in Synthesis Example 2, except that Intermediate 1-(13) was used instead of (4-bromo-phenyl)-diphenyl-amine in the synthesis process of Intermediate 2-a, Intermediate 3-(59) was used instead of Intermediate 2-d in the synthesis process of Intermediate 2-e, and Intermediate 4-(39) was used instead of Intermediate 2-g in the synthesis process of Compound 66. The obtained compound was confirmed by LC-MS and NMR.

C₄₇H₂₂N₄O₁: M+664.23

1H NMR (CDCl₃, 400 MHz) δ (ppm) 8.90 (d, 1H), 8.10 (m, 2H), 7.90-7.00 (m, 24H), 6.59 (d, 1H)

### Synthesis Example 70

### Synthesis of Compound 70

Compound 70 was synthesized in the same manner as in Synthesis Example 2, except that Intermediate 1-(12) was used instead of (4-bromo-phenyl)-diphenyl-amine in the synthesis process of Intermediate 2-a and Intermediate 4-(40) was used instead of Intermediate 2-g in the synthesis process of Compound 66. The obtained compound was confirmed by LC-MS and NMR.

C₃₉H₂₁N₁O₂: M+535.16

1H NMR (CDCl₃, 400 MHz) δ (ppm) 8.93 (d, 1H), 8.12 (m, 2H), 7.88-7.13 (m, 18H)

### Synthesis Example 71

### Synthesis of Compound 71

Compound 71 was synthesized in the same manner as in Synthesis Example 1, except that Intermediate 1-(2) was used instead of 3-iodo-9-phenyl-9H-carbazole in the synthesis process of Intermediate 1-a, Intermediate 2-(41) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₃₃H₂₂S₂: M+450.16

1H NMR (CDCl₃, 400 MHz) δ (ppm) 8.99 (d, 1H), 8.34 (s, 1H), 8.12-8.10 (m, 2H), 7.93 (s, 1H), 7.82 (d, 1H), 7.49-7.13 (m, 11H), 6.83 (d, 2H), 3.73 (s, 3H)

### Synthesis Example 72

### Synthesis of Compound 72

Compound 72 was synthesized in the same manner as in Synthesis Example 1, except that (4-bromo-phenyl)-diphenyl-amine was used instead of 3-iodo-9-phenyl-9H-carbazole in the synthesis process of Intermediate 1-a, Intermediate 2-(42) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₄₂H₃₃N₁O₁: M+567.26

1H NMR (CDCl₃, 400 MHz) δ (ppm) 8.85 (d, 1H), 8.15-8.12 (m, 2H), 7.93-7.82 (m, 3H), 7.49-7.42 (m, 2H), 7.23-7.01 (m, 8H), 6.62-6.46 (m, 8H), 1.40 (s, 9H)

### Synthesis Example 73

### Synthesis of Compound 73

Compound 73 was synthesized in the same manner as in Synthesis Example 1, except that (4-bromo-phenyl)-diphenyl-amine was used instead of 3-iodo-9-phenyl-9H-carbazole in the synthesis process of Intermediate 1-a, Intermediate 2-(23) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₃₉H₂₄N₂O₁: M+536.19

1H NMR (CDCl₃, 400 MHz) δ (ppm) 9.18 (s, 1H), 8.30-7.82 (m, 5H), 7.49-7.01 (m, 10H), 6.62-6.46 (m, 8H)

### Synthesis Example 74

### Synthesis of Compound 74

Compound 74 was synthesized in the same manner as in Synthesis Example 1, except that Intermediate 1-(2) was used instead of 3-iodo-9-phenyl-9H-carbazole in the synthesis process of Intermediate 1-a, Intermediate 2-(44) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₃₂H₂₁NO₂: M+451.16

1H NMR (CDCl₃, 400 MHz) δ (ppm) 8.81 (s, 1H), 8.55 (d, 1H), 8.12-7.82 (m, 7H), 7.49-7.13 (m, 7H), 6.83 (d, 2H), 3.73 (s, 3H)

### Synthesis Example 75

### Synthesis of Compound 75

Compound 75 was synthesized in the same manner as in Synthesis Example 1, except that (4-bromo-phenyl)-diphenyl-amine was used instead of 3-iodo-9-phenyl-9H-carbazole in the synthesis process of Intermediate 1-a, Intermediate 2-(45) was used instead of 2-bromoiodobenzene in the synthesis process of Intermediate 1-c, and Intermediate 3-(75) was used instead of Intermediate 1-d in the synthesis process of Intermediate 1-e. The obtained compound was confirmed by LC-MS and NMR.

C₅₀H₃₁N₁O₂: M+677.24

1H NMR (CDCl₃, 400 MHz) δ (ppm) 8.89 (d, 1H), 8.12-7.01 (m, 22H), 6.62-6.46 (m, 8H)

### Synthesis Example 76

### Synthesis of Compound 76

Compound 76 was synthesized in the same manner as in Synthesis Example 1, except that Intermediate 1-(2) was used instead of 3-iodo-9-phenyl-9H-carbazole in the synthesis process of Intermediate 1-a, Intermediate 2-(46) was used instead of 2-bromoiodobenzene in the synthesis process of Intermediate 1-c, and Intermediate 2-d was used instead of Intermediate 1-d in the synthesis process of Intermediate 1-e. The obtained compound was confirmed by LC-MS and NMR.

C₃₇H₂₄O₂S₁: M+532.15

1H NMR (CDCl₃, 400 MHz) δ (ppm) 9.21 (s, 1H), 8.40 (s, 1H), 8.12 (d, 1H), 7.93 (s, 1H), 7.82 (d, 1H), 7.49-6.83 (m, 16H), 3.73 (s, 3H)

Intermediates 1-(1) through 1-(6), 1-(8), 1-(9), 1-(11) through 1-(13), 1-(17) through 1-(22), and 1-(30)

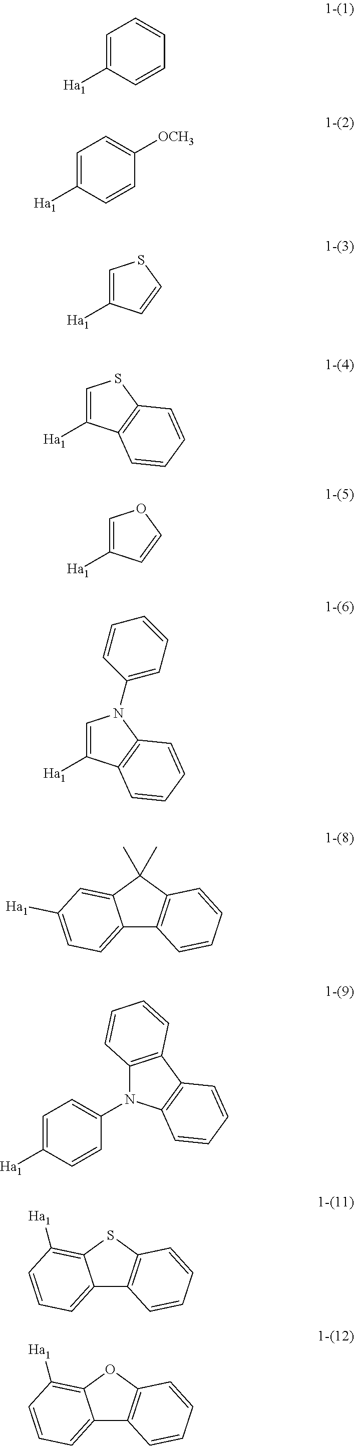

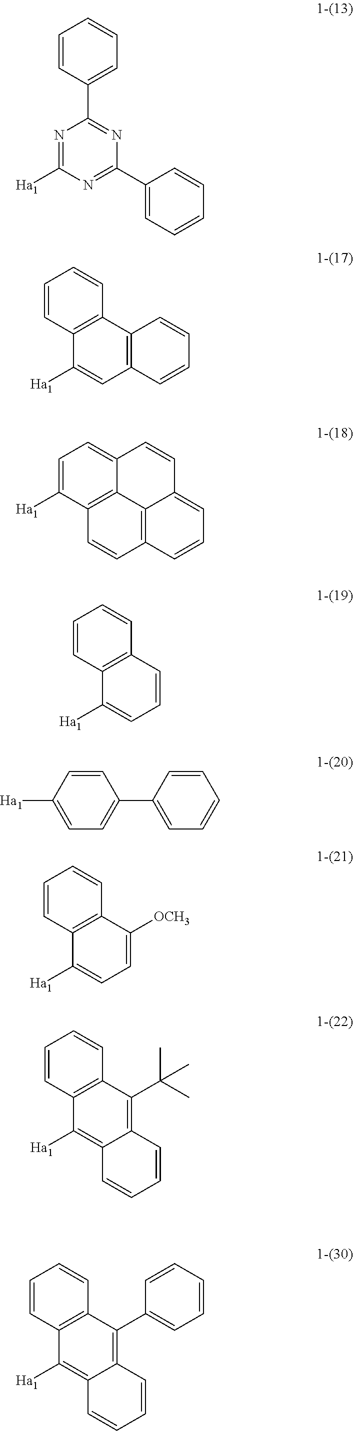

In the Intermediates 1-(1) through 1-(30) above, Ha₁ of Intermediate 1-(21) is I, Ha₁ of Intermediate 1-(13) is Cl, Ha₁ of the other intermediates is Br, and Synthesis Examples of Intermediates 1-(6), 1-(9), 1-(11), 1-(12), and 1-(30) will now be described in detail.

### Synthesis Example of Intermediate 1-(6)

1H-indole

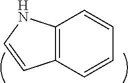

and bromosuccinimide were added to MeCN, the mixture was stirred at 35° C. for a moment, and water was added thereto. Subsequently, the temperature of the mixture was reduced to room temperature, resultant mixture was maintained to induce a reaction therebetween, and the reaction product was then purified.

### Synthesis Example of Intermediate 1-(9)

Carbazole

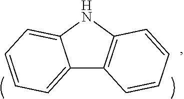

1-bromo-4-iodobenzene, tris(dibenzylideneacetone)dipalladium(0) (Pd₂(dba)₃), sodium tert-butoxide, and tri-tert-butyl phosphine (P(t-Bu)₃) were added to toluene and the mixture was stirred by reflux at 100° C.# for 5 hours. Then, the reaction product was purified to obtain Intermediate 1-(9).

### Synthesis Example of Intermediate 1-(11)

Dibenzothiophen

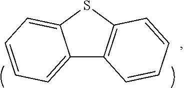

n-BuLi, and 1,2-dibromobenzene were added to THF, the mixture was stirred by reflux, and the reaction product was purified to obtain Intermediate 1-(11).

### Synthesis Example of Intermediate 1-(12)

Dibenzofuran

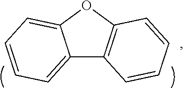

n-BuLi, and 1,2-dibromobenzene were added to THF, the mixture was stirred by reflux, and the reaction product was purified to obtain Intermediate 1-(12).

### Synthesis Example of Intermediate 1-(30)

9,10-dibromoanthracene

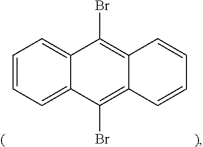

bromobenaene, Pd(PPh₃)₄, and K₂CO₃ were added to THF, the mixture was stirred by reflux, and the reaction product was purified to obtain Intermediate 1-(30).

Intermediates 2-(23), 2-(24), 2-(26), 2-(28), 2-(41), 2-(42), 2-(44), 2-(45), and 2-(46)

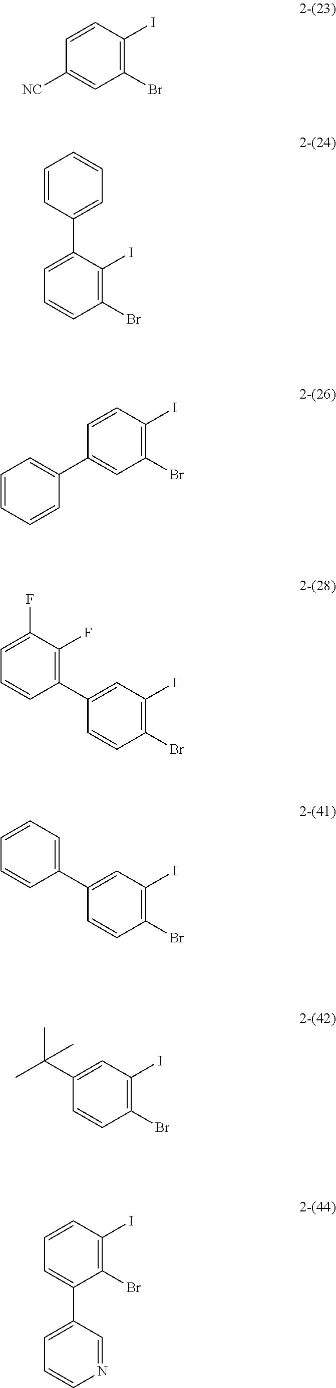

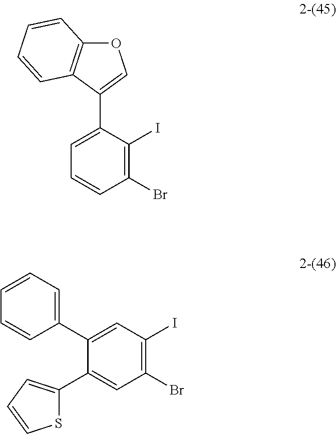 [SMILES: Brc1cccc(-c2coc3ccccc23)c1I ; Brc1cc(-c2cccs2)c(-c2ccccc2)cc1I]

Intermediates 3-(15), 3-(16), 3-(20), 3-(31), 3-(59), and 3-(75)

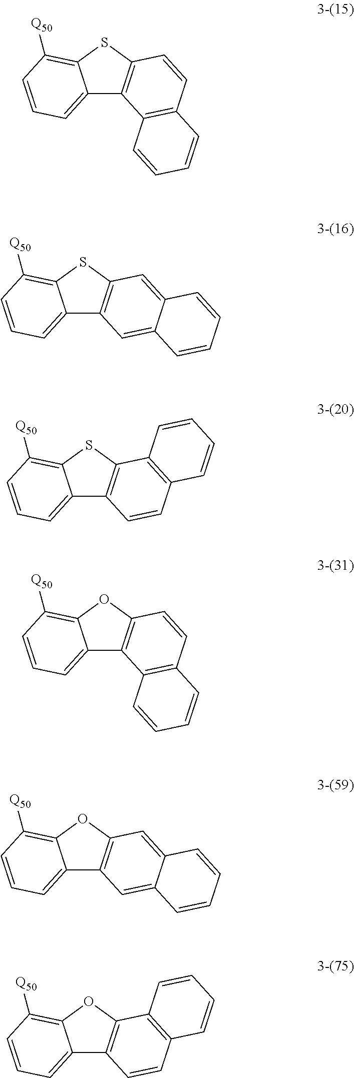

In this regard, Q₅₀ of Intermediates 3-(15), 3-(16), 3-(20), 3-(31), 3-(59), and 3-(75) is a borate group.

Intermediates 4-(14) through 4-(16), 4-(26), 4-(33), 4-(34), 4-(37) through (4-(40)

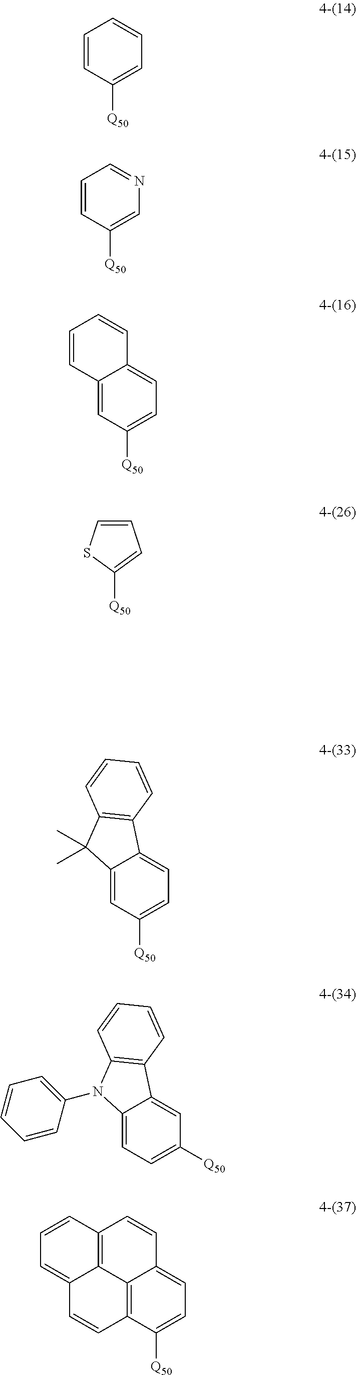

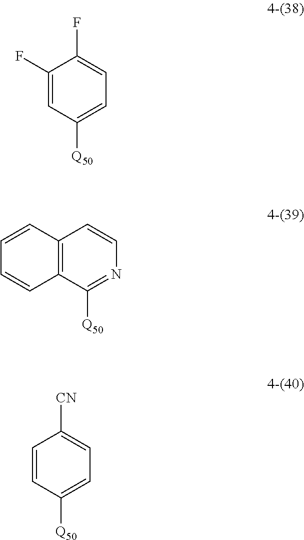

Q₅₀ of each of the Intermediates 4-(14), 4-(26) and 4-(40) is a boric acid group, and Q₅₀ of each of the Intermediates 4-(15), 4-(16), 4-(33), 4-(34), 4-(37), 4-(38), and 4-(39) is a borate group.

### Comparative Synthesis Example A

### Synthesis of Comparative Compound 3-f

Comparative Compound 3-f was synthesized according to Comparative Reaction Scheme A below:

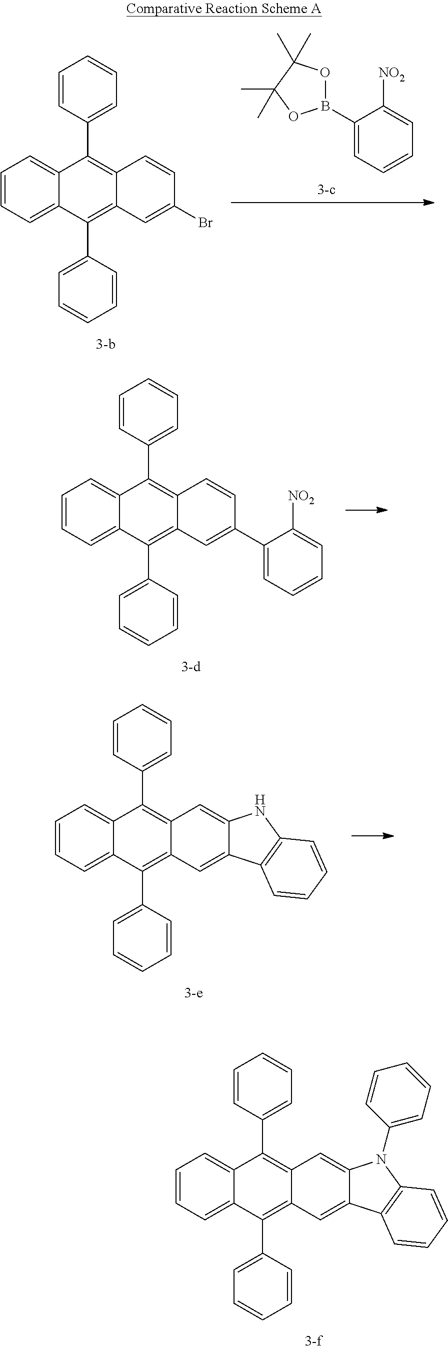 [SMILES: Brc1ccc2c(-c3ccccc3)c3ccccc3c(-c3ccccc3)c2c1 ; CC1(C)OB(c2ccccc2[N+](=O)[O-])OC1(C)C ; O=[N+]([O-])c1ccccc1-c1ccc2c(-c3ccccc3)c3ccccc3c(-c3ccccc3)c2c1 ; c1ccc(-c2c3ccccc3c(-c3ccccc3)c3cc4c(cc23)[nH]c2ccccc24)cc1 ; c1ccc(-c2c3ccccc3c(-c3ccccc3)c3cc4c(cc23)c2ccccc2n4-c2ccccc2)cc1]

### Synthesis of Intermediate 3-d

2.0 g of Intermediate 3-b (available from Tokyo Chemical Industry Col, Ltd.), 1.5 g (1.2 eq) of Intermediate 3-c, 283 mg (0.05 eq) of Pd(PPh₃)₄, and 3.38 g (5.0 eq) of K₂CO₃ were dissolved in 50 ml of THF and 15 ml of distilled water, the temperature of the mixed solution was raised to 120° C., and the mixed solution was then stirred by reflux for 24 hours. The obtained reaction product was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 1.57 g of Intermediate 3-d (yield 71%). The obtained compound was confirmed by LC-MS.

C₃₂H₂₁N₁O₂: M+451.16

### Synthesis of Intermediate 3-e

1.5 g of Intermediate 3-d and 3.57 g (5.0 eq) of PPh₃ were added to 50 ml of o-dichlorobenzene and the mixture was then stirred by reflux at 180 to 200° C.# overnight. The obtained reaction product was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 1.57 g of Intermediate 3-e (yield 71%). The obtained compound was confirmed by LC-MS.

C₃₂H₂₁N₁: M+419.17

### Synthesis of Compound 3-f

1.5 g of Intermediate 3-e, 1.1 g of iodobenzene, 34 mg (0.05 eq) of CuI, 2.47 g (5.0 eq) of K₂CO₃, and 47 mg (0.05 eq) of 18-crown-6 were added to 100 ml of DMF and the mixture was stirred by reflux at 130° C.# overnight. The obtained reaction product was cooled down to room temperature and then extracted three times with 100 ml of water and 100 ml of diethylether. The obtained organic layer was dried with magnesium sulfate and a solvent was evaporated therefrom to obtain a crude product. The crude product was purified with silicagel column chromatography to obtain 1.28 g of Comparative Compound 3-f (yield 72%). The obtained compound was confirmed by LC-MS and NMR.

C₃₈H₂₅N₁: M+495.20

1H NMR (CDCl₃, 400 MHz) δ (ppm) 7.91 (m, 4H), 7.55-7.00 (m, 21H)

### Comparative Synthesis Example B

### Synthesis of Comparative Compound 4-d

Comparative Compound 4-d was synthesized according to Comparative Reaction Scheme B below:

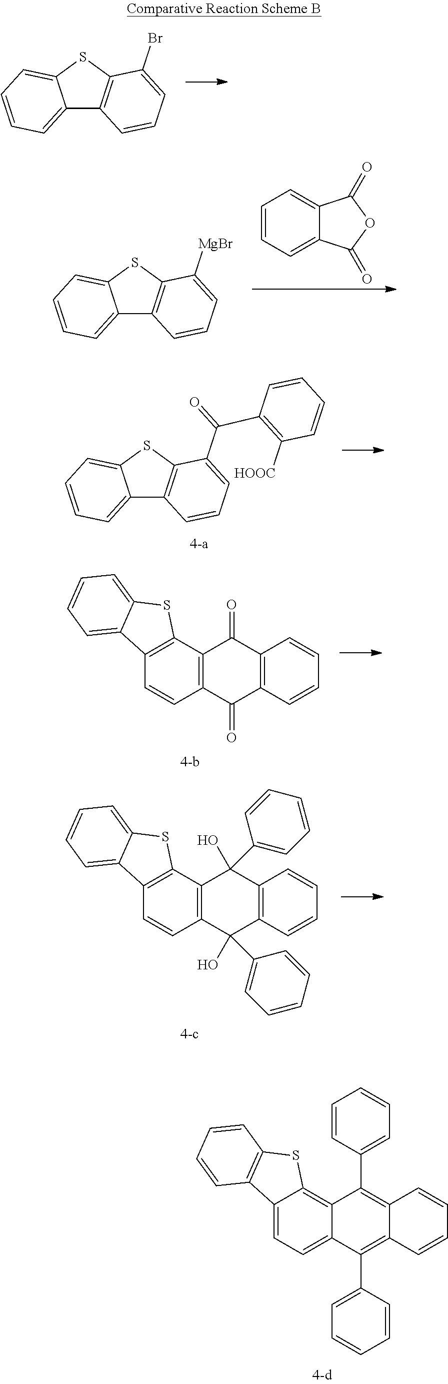

### Synthesis of Intermediate 4-a

1.2 g (1.3 eq) of Mg and 1.93 g (0.2 eq) of I₂ were added to 70 ml of THF and the mixture was stirred by reflux for 1 hour. The reaction solution was cooled down to room temperature, 10 g (1.0 eq) of 4-bromodibenzothiophene dissolved in 500 ml of THF was added to the reaction solution, and the resultant reaction solution was then stirred by reflux for 1 hour. The reaction solution was cooled down to room temperature, 5.63 g (1.0 eq) of phthalic anhydride was added thereto, and the resultant reaction solution was stirred at 50° C.# for 5 hours. The obtained reaction solution was cooled down to room temperature and then extracted three times with 200 ml of water and 200 ml of dichloromethane. The obtained organic layer was dried with magnesium sulfate and a solvent was evaporated therefrom to obtain a crude product. The crude product was purified with silicagel column chromatography to obtain 9.35 g of Intermediate 4-a (yield 74%). The obtained compound was confirmed by LC-MS.

C₂₀H₁₂O₃S₁: M+332.05

### Synthesis of Intermediate 4-b

9.0 g of Intermediate 4-a was mixed with 70 ml of methanesulfonic acid and the mixture was stirred at 100° C.# for 2 hours. The reaction solution was cooled down to room temperature and 100 ml of distilled water was then added thereto to obtain a solid. The solid was filtered and then washed with an aqueous NaOH solution to obtain a crude product.

The crude product was purified with silicagel column chromatography to obtain 3.83 g of Intermediate 4-b (yield 45%). The obtained compound was confirmed by LC-MS.

C₂₀H₁₀O₂S₁: M+314.04

### Synthesis of Intermediate 4-c

5.86 g (3.0 eq) of 2-bromonaphthalene was dissolved in 100 ml of THF and (1.2 eq) of n-BuLi was slowly added to the mixture at −78° C. The resultant mixture was stirred for 1 hour, 3.0 g (1.0 eq) of Intermediate 4-b was added thereto, the temperature of the mixture was raised to room temperature, and the resultant mixture was stirred for 6 hours. The reaction solution was extracted with distilled water and dichloromethane. The obtained organic layer was dried with magnesium sulfate and a solvent was evaporated therefrom to obtain a crude product. The crude product was purified with silicagel column chromatography to obtain 4.38 g of Intermediate 4-c (yield 98%). The obtained compound was confirmed by LC-MS.

C₃₂H₂₂O₂S₁: M+470.13

### Synthesis of Comparative Compound 4-d

4.0 g of Intermediate 4-c, 4.23 g (3.0 eq) of Kl, and 5.4 g (6.0 eq) of NaH₂PO₂H₂O were mixed with 100 ml of acetic acid and the mixture was stirred by reflux for 6 hours. The reaction solution was cooled down to room temperature and distilled water was added thereto. The obtained solid was then filtered. The filtered solid was washed with an aqueous NaOH solution to make it neutral. The resultant product was purified with silicagel column chromatography to obtain 2.15 g of Intermediate 4-d (yield 57%). The obtained compound was confirmed by LC-MS and N MR.

C₃₂H₂₀S₁: M+436.13

1H NMR (CDCl₃, 400 MHz) δ (ppm) 7.91-7.78 (m, 5H), 7.48-7.22 (m, 15H)

### Example 1

As an anode, a substrate formed of ITO/Ag/ITO (70 Å/1000 Å/70 Å) layers was cut to a size of 50 mm×50 mm×0.7 mm, washed with ultrasonic waves in isopropyl alcohol and pure water for 5 minutes each, and then cleaned with UV and ozone for 30 minutes. Subsequently, the glass substrate was mounted on a vacuum deposition device.

2-TNATA was deposited on the ITO layer to form a HIL having a thickness of 600 Å and 4,4′-bis[N-(1-naphthyl)-N-phenylamino]biphenyl (NPB) was then deposited on the HIL to form a HTL having a thickness of 1,000 Å.

Next, Compound 10 (acting as a green phosphorescent host) and Ir(ppy)₃ (acting as a green phosphorescent dopant) were co-deposited on the HTL at a weight ratio of 91:9 to form an EML having a thickness of 250 Å.

Thereafter, BCP was deposited on the EML to form a HBL having a thickness of 50 Å, Alq₃ was deposited on the HBL to form an ETL having a thickness of 350 Å, LiF was deposited on the ETL to form an EIL having a thickness of 10 Å, and Mg and Ag were co-deposited on the EIL at a weight ratio of 90:10 to form a second electrode (cathode) having a thickness of 120 Å, thereby completing the manufacture of an OLED (green light emission).

### Example 2

An OLED was manufactured in the same manner as in Example 1, except that Compound 12 was used instead of Compound 10 in the formation of the EML.

### Example 3

As an anode, a substrate formed of ITO/Ag/ITO (70 Å/1000 Å/70 Å) layers was cut to a size of 50 mm×50 mm×0.7 mm, washed with ultrasonic waves in isopropyl alcohol and pure water for 5 minutes each, and then cleaned with UV and ozone for 30 minutes. Subsequently, the glass substrate was mounted on a vacuum deposition device.

2-TNATA was deposited on the ITO layer to form a HIL having a thickness of 600 Å and NPB was then deposited on the HIL to form a HTL having a thickness of 1,350 Å.

Next, Compound 7 (acting as a red phosphorescent host) and PtOEP (acting as a red phosphorescent dopant) were co-deposited on the HTL at a weight ratio of 94:6 to form an EML having a thickness of 400 Å.

Thereafter, BCP was deposited on the EML to form a HBL having a thickness of 50 Å, Alq₃ was deposited on the HBL to form an ETL having a thickness of 350 Å, LiF was deposited on the ETL to form an EIL having a thickness of 10 Å, and Mg and Ag were co-deposited on the EIL at a weight ratio of 90:10 to form a second electrode (cathode) having a thickness of 120 Å, thereby completing the manufacture of an OLED (red light emission).

### Example 4

An OLED was manufactured in the same manner as in Example 3, except that Compound 64 was used instead of Compound 7 in the formation of the EML.

### Example 5

An OLED was manufactured in the same manner as in Example 3, except that Compound 66 was used instead of Compound 7 in the formation of the EML.

### Example 6

An OLED was manufactured in the same manner as in Example 1, except that CBP was used instead of Compound 10 in the formation of the EML and Compound 36 was used instead of Alq₃ in the formation of the ETL.

### Example 7

An OLED was manufactured in the same manner as in Example 6, except that Compound 39 was used instead of Compound 36 in the formation of the ETL.

### Example 8

An OLED was manufactured in the same manner as in Example 3, except that Compound 36 was used instead of Alq₃ in the formation of the ETL.

### Example 9

An OLED was manufactured in the same manner as in Example 8, except that Compound 64 was used instead of Compound 7 in the formation of the EML and Compound 39 was used instead of Compound 36 in the formation of the ETL.

### Comparative Example 1

An OLED was manufactured in the same manner as in Example 1, except that CBP was used instead of Compound 10 in the formation of the EML.

### Comparative Example 2

An OLED was manufactured in the same manner as in Example 3, except that CBP was used instead of Compound 7 in the formation of the EML.

### Comparative Example 3

An OLED was manufactured in the same manner as in Example 1, except that Comparative Compound 3-f was used instead of Compound 10 in the formation of the EML.

### Comparative Example 4

An OLED was manufactured in the same manner as in Example 1, except that Comparative Compound 4-d was used instead of Compound 10 in the formation of the EML.

### Evaluation Example 1

Driving voltage, current density, brightness, emission color, efficiency, and half lifetime (@100 mA/cm²) of each of the OLEDs of Examples 1 through 9 and Comparative Examples 1 through 4 were evaluated using PR650 Spectroscan Source Measurement Unit (available from PhotoResearch), and the results are shown in Table 1 below.

TABLE 1

| | | | | Driving | Current | | | | Color | Life |
|---|---|---|---|---|---|---|---|---|---|---|
| | EML | | | voltage | density | Brightenss | Efficiency | Emission | coordiante | time |
| | host | dopant | ETL | (V) | (mA/cm²) | (cd/m²) | (cd/A) | color | (X, Y) | (hr) |
| Example 1 | Compound 10 | Ir(ppy)₃ | Alq₃ | 5.7 | 10 | 6,521 | 65.2 | green | 0.21 | 77 |
| | | | | | | | | | 0.72 | |
| Example 2 | Compound 12 | Ir(ppy)₃ | Alq₃ | 5.2 | 10 | 6,180 | 61.8 | green | 0.30 | 84 |
| | | | | | | | | | 0.66 | |
| Example 3 | Compound 7 | PtOEP | Alq₃ | 5.9 | 10 | 3,042 | 30.4 | red | 0.65 | 121 |
| | | | | | | | | | 0.34 | |
| Example 4 | Compound 64 | PtOEP | Alq₃ | 6.3 | 10 | 3,407 | 34.0 | red | 0.64 | 125 |
| | | | | | | | | | 0.35 | |
| Example 5 | Compound 66 | PtOEP | Alq₃ | 6.4 | 10 | 3,211 | 32.1 | red | 0.64 | 120 |
| | | | | | | | | | 0.36 | |
| Example 6 | CBP | Ir(ppy)₃ | Compound 36 | 5.3 | 10 | 5,967 | 59.7 | green | 0.24 | 104 |
| | | | | | | | | | 0.70 | |
| Example 7 | CBP | Ir(ppy)₃ | Compound 39 | 5.5 | 10 | 6,429 | 64.3 | green | 0.24 | 98 |
| | | | | | | | | | 0.72 | |
| Example 8 | Compound 7 | PtOEP | Compound 36 | 5.2 | 10 | 5,107 | 41.1 | red | 0.64 | 134 |
| | | | | | | | | | 0.32 | |
| Example 9 | Compound 64 | PtOEP | Compound 39 | 5.8 | 10 | 4,852 | 38.5 | red | 0.67 | 129 |
| | | | | | | | | | 0.36 | |
| Comparative | CBP | Ir(ppy)₃ | Alq₃ | 6.8 | 10 | 4,766 | 47.7 | green | 0.25 | 61 |
| Example 1 | | | | | | | | | 0.70 | |
| Comparative | CBP | PtOEP | Alq₃ | 7.3 | 10 | 2,212 | 22.1 | red | 0.67 | 89 |
| Example 2 | | | | | | | | | 0.32 | |
| Comparative | Comparative | Ir(ppy)₃ | Alq₃ | 7.4 | 10 | 3,109 | 31.1 | green | 0.26 | 43 |
| Example 3 | Compound 3-f | | | | | | | | 0.70 | |
| Comparative | Comparative | Ir(ppy)₃ | Alq₃ | 7.7 | 10 | 3,005 | 30.1 | green | 0.27 | 40 |
| Example 4 | Compound 4-d | | | | | | | | 0.70 | |

In Table 1, the lifetime data were obtained by measuring time at which the luminance of each of the OLEDs is decreased to 97% of the initial luminance (luminance measurement conditions: constant current of 10 mA/cm²).

From the results shown in Table 1, it is confirmed that each of the OLEDs of Examples 1 through 9 has excellent driving voltage, higher luminance, higher efficiency, higher color purity, and longer lifetime, as compared to the OLEDs of Comparative Examples 1 through 4.

As described above, according to the one or more embodiments of the present invention, an OLED including the condensed-cyclic compound of Formula 1 may have low driving voltage, high luminance, high efficiency, and long lifetime.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A condensed-cyclic compound represented by Formula 1 below: 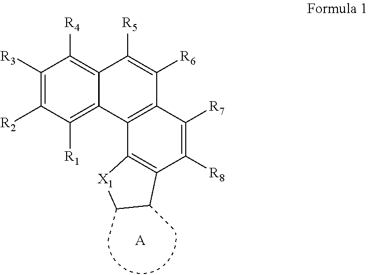
wherein X₁ is N(R₁₀), S, or O;
a ring A is a substituted or unsubstituted aromatic ring;
R₁ through R₈ and R₁₀ are each independently hydrogen, deuterium, a halogen atom, a hydroxyl group, a cyano group, a nitro group, an amino, group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —Si(R₂₁)(R₂₂)(R₂₃), or —N(R₂₄)(R₂₅); and
R₂₁ through R₂₅ are each independently a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

2. The condensed-cyclic compound of claim 1, wherein X₁ is S or O.

3. The condensed-cyclic compound of claim 1, wherein the ring A is a substituted or unsubstituted benzene ring or a substituted or unsubstituted naphthalene ring.

4. The condensed-cyclic compound of claim 1, wherein the condensed-cyclic compound is represented by one of Formulae 1A, 1B, 1C, or 1D below: 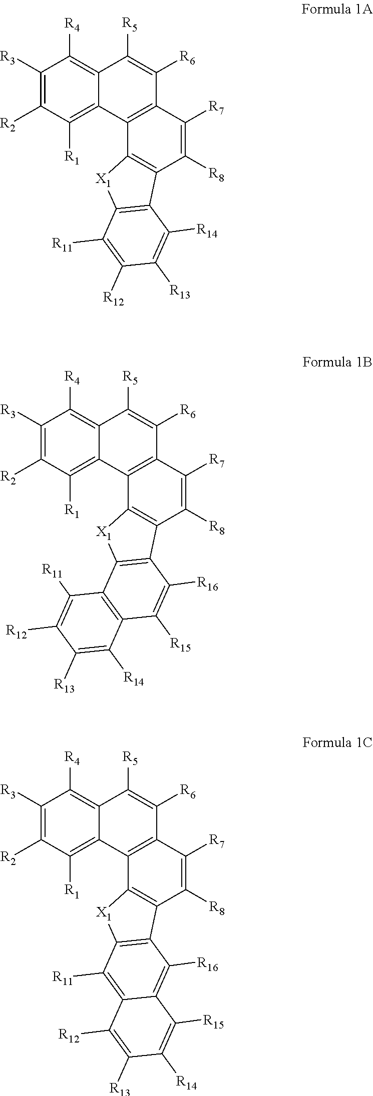 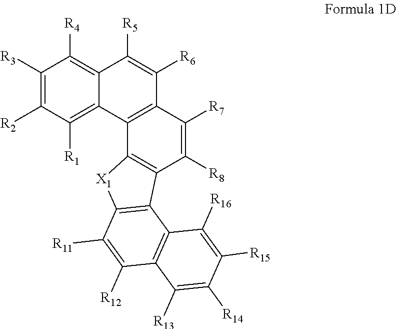
wherein X₁ and R₁ through R₈ are the same as defined in claim 1; and
R₁₁ through R₁₆ are each independently the same as defined in claim 1 for R₁.

5. The condensed-cyclic compound of claim 4, wherein R₁ through. R₈ and R₁₀ through R₁₆ are each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₁₀ alkyl group, a substituted or unsubstituted, C₁-C₁₀ alkoxy group, —N(R₂₄)(R₂₅) (wherein R₂₄ and R₂₅ are each independently one selected from a phenyl group; a naphthyl group; an anthryl group; and a phenyl group; a naphthyl group; and an anthryl group that is substituted with at least one of deuterium, a cyano group, a halogen atom, CH₂F, CHF₂, and CF₃), and Formulae 2A through 2P below: 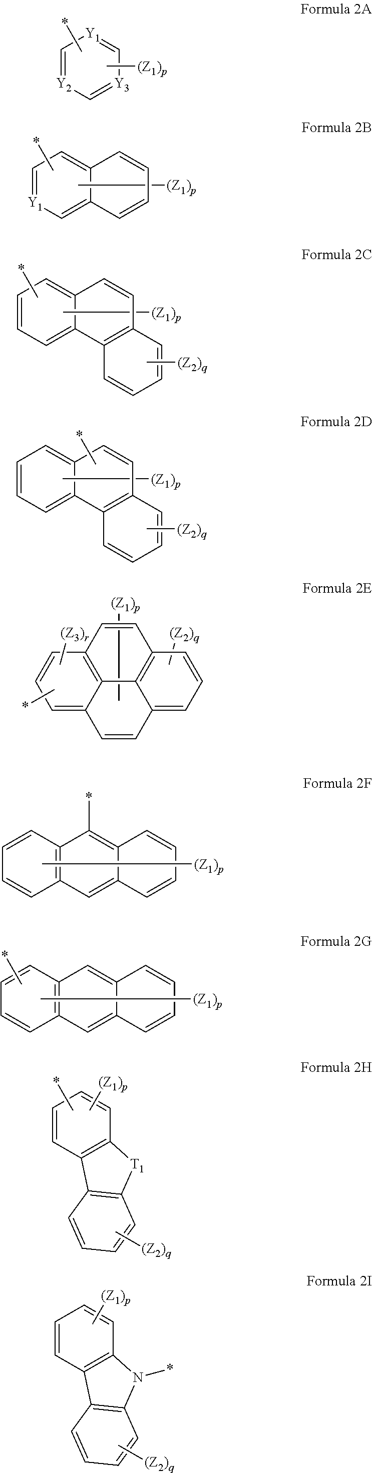 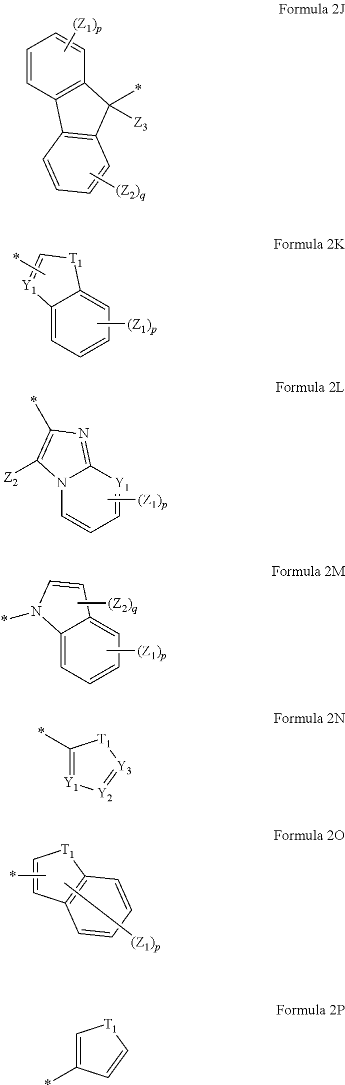
wherein Y₁ through Y₃ are each independently ═N or ═C(Z₁₁)—;
T₁ is —S—, —O—, —N(Z₁₂)—, or —C(Z₁₃)(Z₁₄)—;
Z₁ through Z₃ and Z₁₁ through Z₁₄ are each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a C₁-C₆₀ alkyl group; a C₂-C₆₀ alkenyl group; a C₂-C₆₀ alkynyl group; a C₁-C₆₀ alkoxy group; a C₃-C₆₀ cycloalkyl group; a C₅-C₆₀ aryl group; a C₅-C₆₀ aryloxy group; a C₅-C₆₀ arylthio group; a C₂-C₆₀ heteroaryl group; a C₁-C₆₀ alkyl group, a C₂-C₆₀ alkenyl group, a C₂-C₆₀ alkynyl group, a C₁-C₆₀ alkoxy group, a C₃-C₆₀ cycloalkyl group, a C₅-C₆₀ aryl group, a C₅-C₆₀ aryloxy group, a C₅-C₆₀ arylthio group, and a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group; —N(Q₁)(Q₂); or —Si(Q₃)(Q₄)(Q₅);
Q₁ through Q₅ are each independently one of a C₃-C₆₀ cycloalkyl group; a C₅-C₆₀ aryl group; a C₅-C₆₀ aryloxy group; a C₅-C₆₀ arylthio group; a C₂-C₆₀ heteroaryl group; and a C₁-C₆₀ alkyl group, a C₂-C₆₀ alkenyl group, a C₂-C₆₀ alkynyl group, a C₁-C₆₀ alkoxy group, a C₃-C₆₀ cycloalkyl group, a C₅-C₆₀ aryl group, a C₅-C₆₀ aryloxy group, a C₅-C₆₀ arylthio group, and a C₂-C₆₀ heteroaryl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
p is an integer of 1 to 9;
q is an integer of 1 to 4; and
r is an integer of 1 to 3.

6. The condensed-cyclic compound of claim 5, wherein Z₁ through Z₃ and Z₁₁ through Z₁₄ are each independently one of hydrogen; deuterium; a halogen atom; a hydroxyl group; a cyano group; a nitro group; an amino group; an amidino group; hydrazine; hydrazone; a carboxyl group or a salt thereof; a sulfonic acid group or a salt thereof; a phosphoric acid or a salt thereof; a methyl group; an ethyl group; a propyl group; a butyl group; a pentyl group; a methoxy group; an ethoxy group; a propoxy group; a butoxy group; a pentoxy group; a methyl group, an ethyl group, a propyl group, a butyl group, a pentyl group, a methoxy group, an ethoxy group, a propoxy group, butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof; a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group; a carbazolyl group; an imidazolyl group; an imidazolinyl group; an imidazopyridinyl group; an imidazopyrimidinyl group; a pyridinyl group; a pyrimidinyl group; a triazinyl group; a quinolinyl group; a benzoimidazolyl group; a phenyl-benzoimidazolyl group; a carbazolyl group, an imidazolyl group, an imidazolinyl group, an imidazopyridinyl group, an imidazopyrimidinyl group, a pyridinyl group, a pyrimidinyl group, and a triazinyl group, a quinolinyl group, a benzoimidazolyl group, and a phenyl-benzoimidazolyl group that is substituted, with at least one of deuterium, a halogen atom, a hydroxyl group, a cyano group, a nitro, group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group; and —N(Q₁)(Q₂) wherein Q₁ and Q₂ are each independently one of a phenyl group; a naphthyl group; a fluorenyl group; a phenanthrenyl group; an anthryl group; a pyrenyl group; a chrysenyl group; a phenyl group, a naphthyl group, a fluorenyl group, a phenanthrenyl group, an anthryl group, a pyrenyl group, and a chrysenyl group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid, group or a salt thereof, a phosphoric acid or a salt thereof, a methyl group, an ethyl group, a propyl group, a butyl group, a pentyl group, a methoxy group, an ethoxy group, a propoxy group, a butoxy group, and a pentoxy group.

7. The condensed-cyclic compound: of claim 4, wherein R₁ through R₈ and R₁₀ through R₁₆ are each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, a substituted or unsubstituted phenyl group, a substituted or unsubstituted naphthyl group, a substituted or unsubstituted fluorenyl group, a substituted or unsubstituted phenanthrenyl group, a substituted or unsubstituted anthryl group, a substituted or unsubstituted pyrenyl group, a substituted or unsubstituted pyridinyl group, a substituted or unsubstituted indolyl group, a substituted or unsubstituted carbazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted dibenzothiophenyl group, a substituted or unsubstituted dibenzofuranyl group, or a substituted or unsubstituted triazinyl group.

8. The condensed-cyclic compound of claim 4, wherein R₁ through R₈ and R₁₀ through R₁₆ are each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, and Formulae 3-1 through 3-40 below: 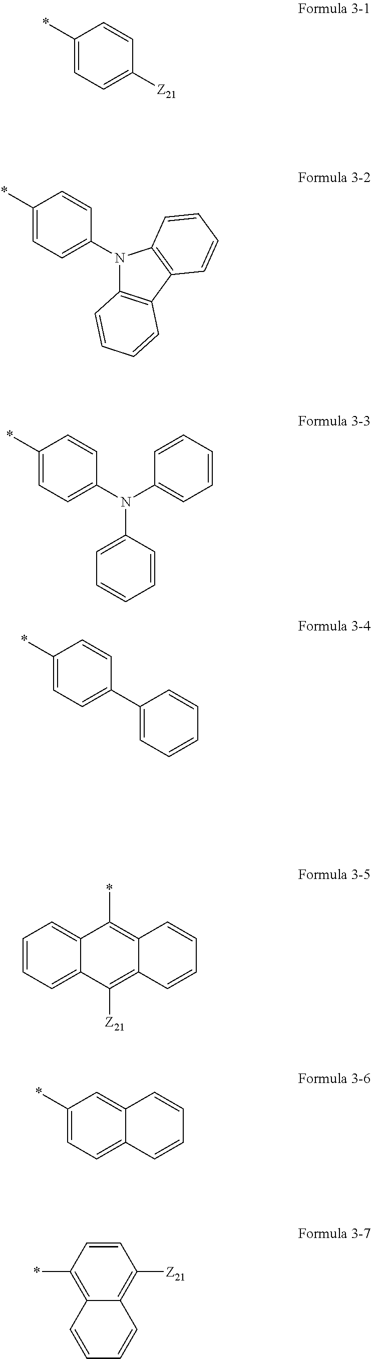 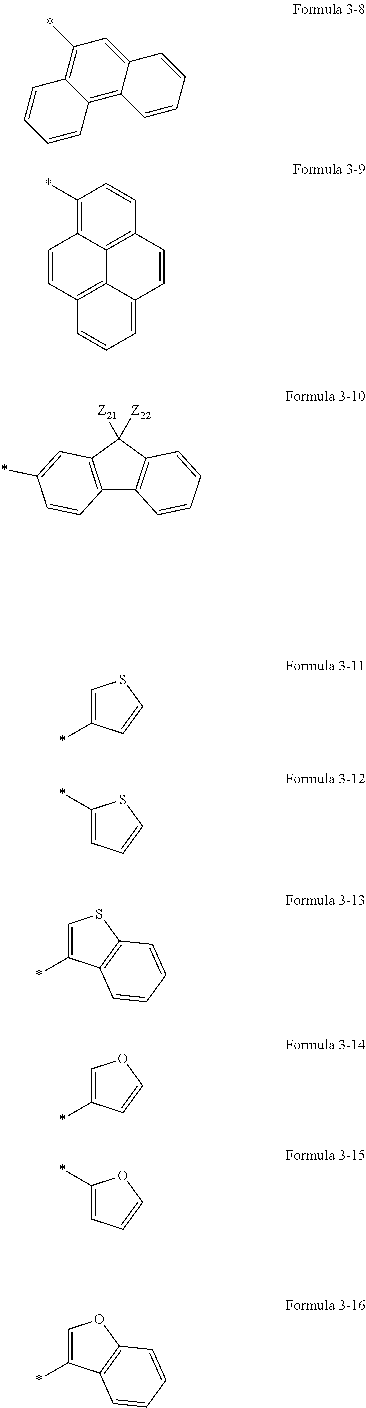 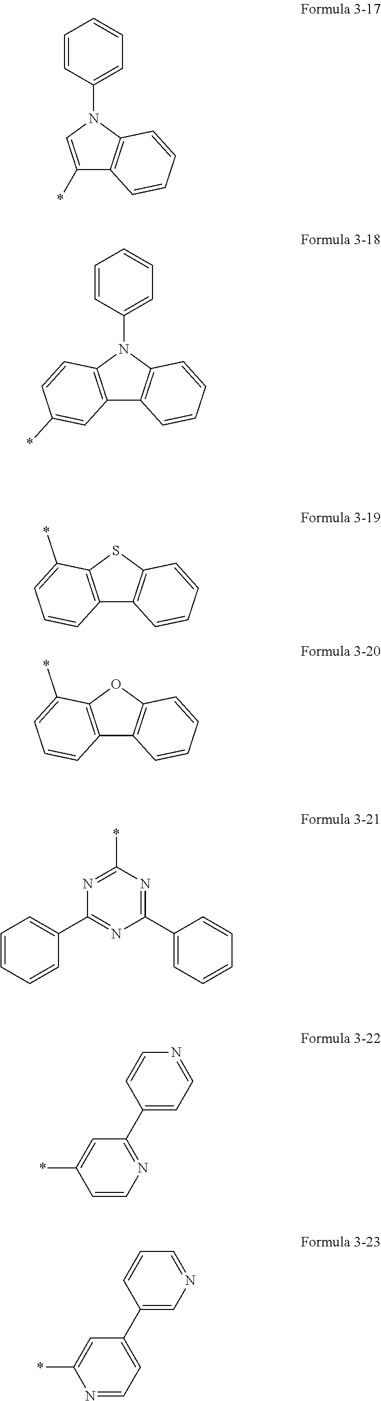 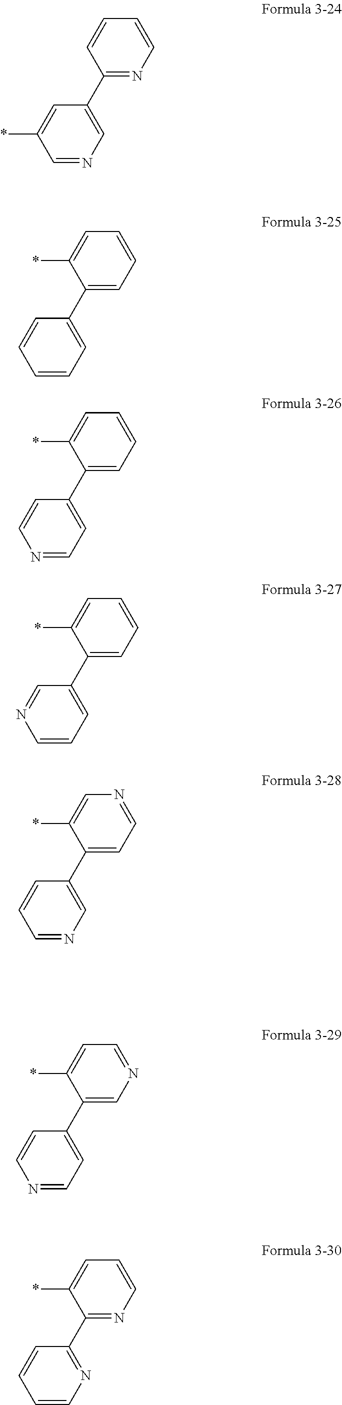 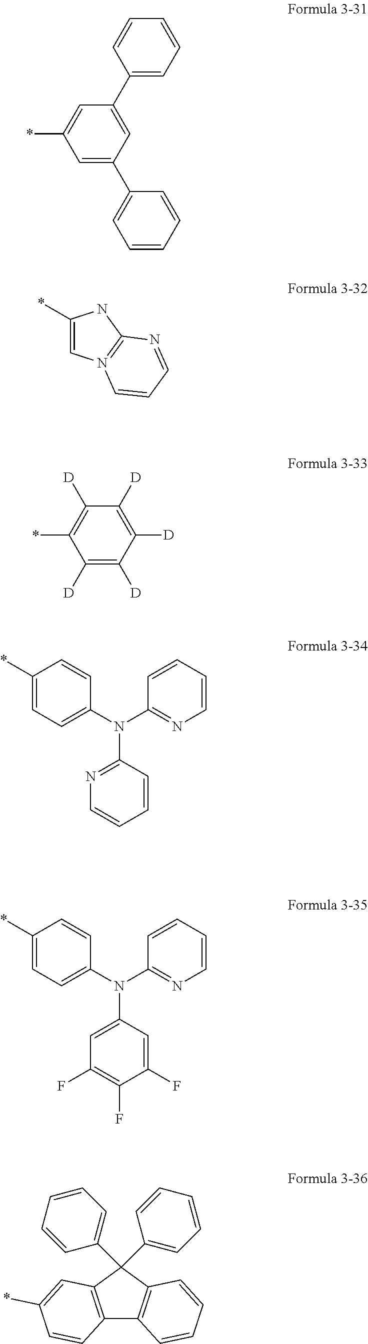 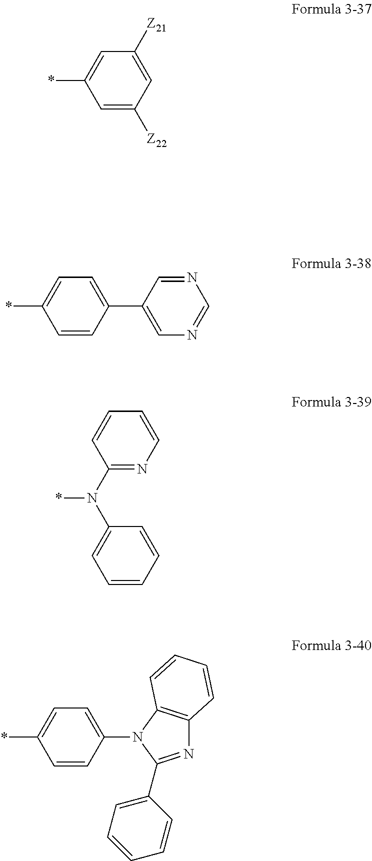
wherein Z₂₁ and Z₂₂ are each independently one selected from hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, a pentoxy group; and a methyl group, an ethyl group, a propyl group, a butyl group, a pentyl group, a methoxy group, an ethoxy group, a propoxy group, a butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof.

9. The condensed-cyclic compound of claim 1, wherein the condensed-cyclic compound is represented by one of Formulae 1A-1 through 1D-3 below: 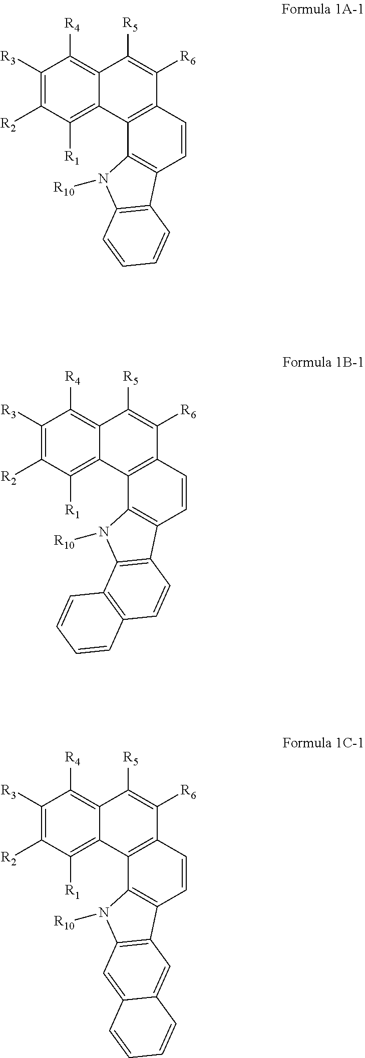 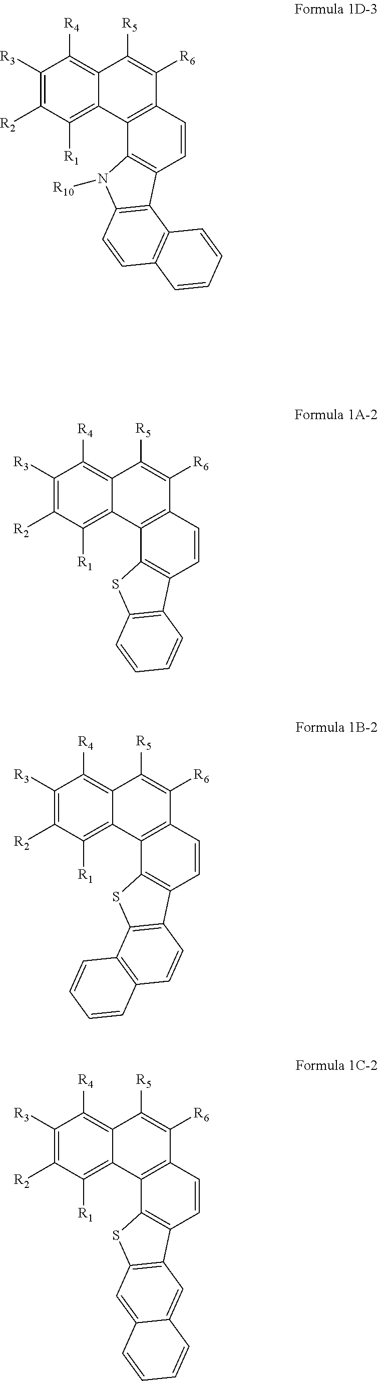 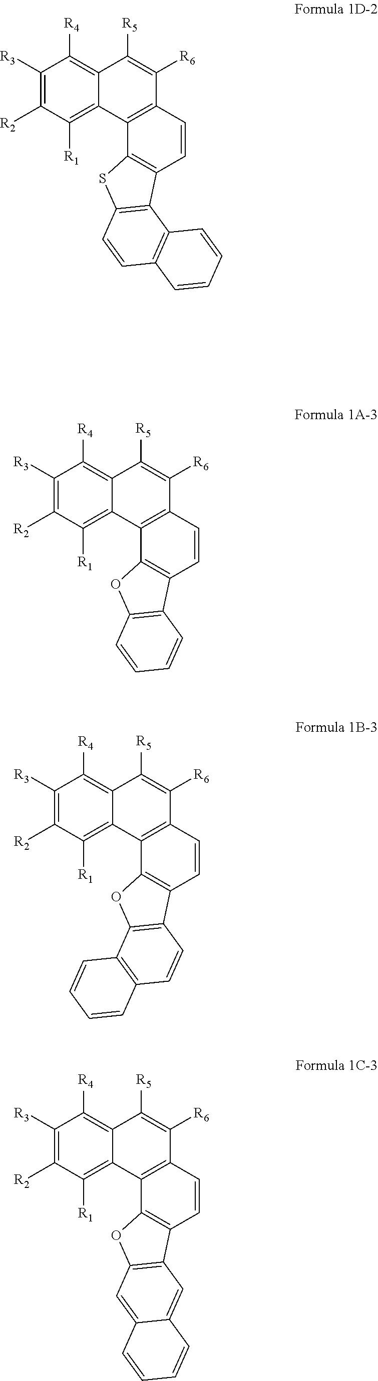 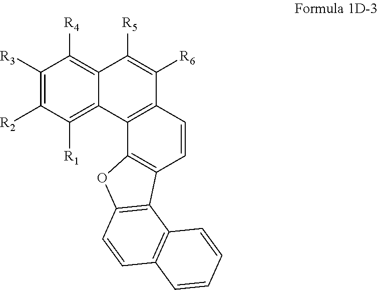
wherein R₁ through R₆ and R₁₀ are the same as defined in claim 1.

10. The condensed-cyclic compound of claim 9, wherein R₁ through R₆ and R₁₀ are each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted phenyl group, a substituted or unsubstituted naphthyl group, a substituted or unsubstituted fluorenyl group, a substituted or unsubstituted phenanthrenyl group, a substituted or unsubstituted anthryl group, a substituted or unsubstituted pyrenyl group, a substituted or unsubstituted pyridinyl group, a substituted or unsubstituted indolyl group, a substituted or unsubstituted carbazolyl group, a substituted or unsubstituted furanyl group, a substituted or unsubstituted benzofuranyl group, a substituted or unsubstituted thiophenyl group, a substituted or unsubstituted benzothiophenyl group, a substituted or unsubstituted dibenzothiophenyl group, a substituted or unsubstituted, dibenzofuranyl group, or a substituted or unsubstituted triazinyl group.

11. The condensed-cyclic compound of claim 9, wherein R₁ through R₆ and R₁₀ are each independently one of hydrogen, deuterium, a halogen atom, a hydroxyl group, a cyano group, a nitro group, an amino group, an amidino group, hydrazine, hydrazone, a carboxyl group or a salt thereof a sulfonic acid group or a salt thereof, a phosphoric acid or a salt thereof, a C₁-C₁₀ alkyl group, a C₁-C₁₀ alkoxy group, and Formulae 3-1 through 3-40 below: 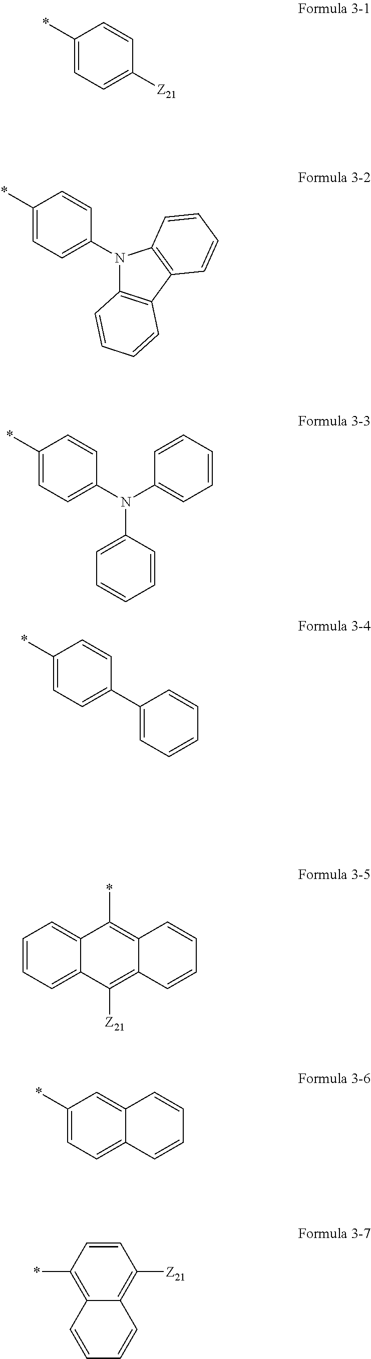 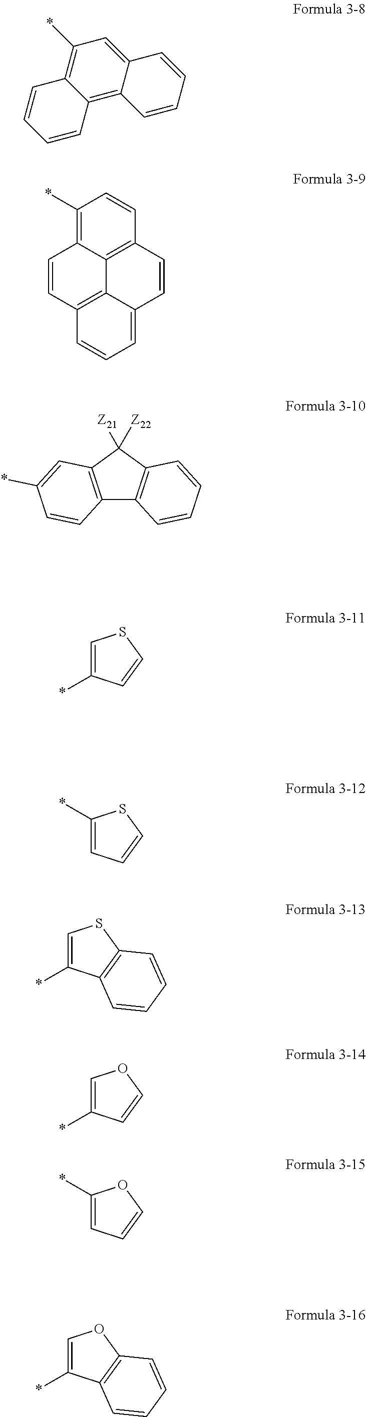 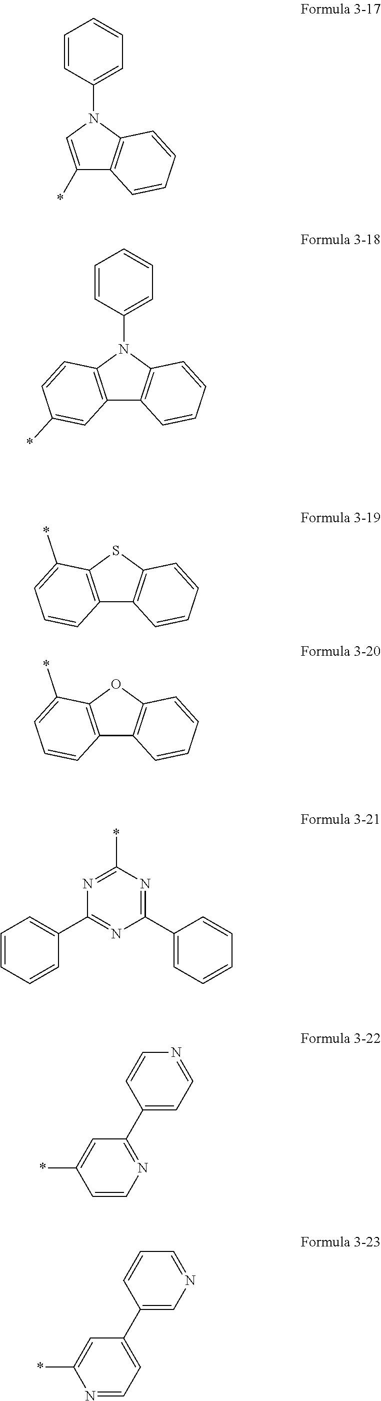 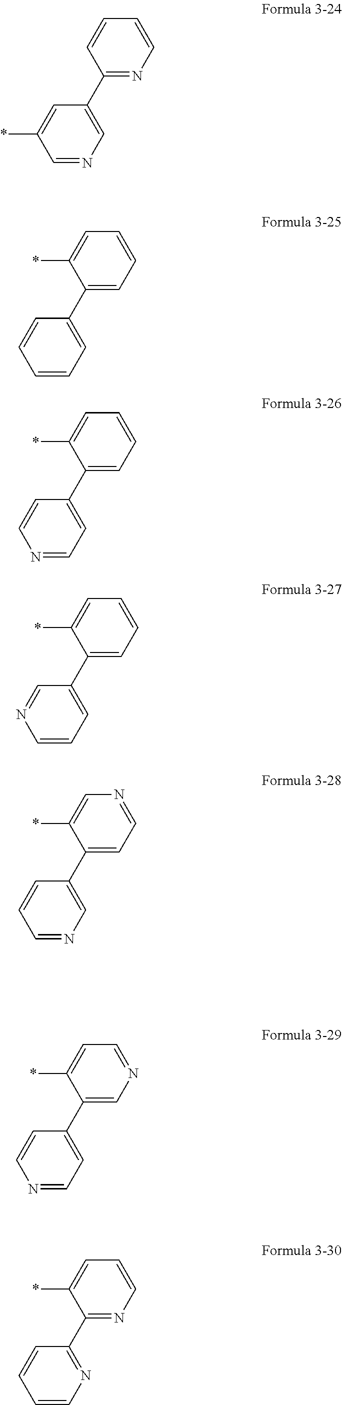 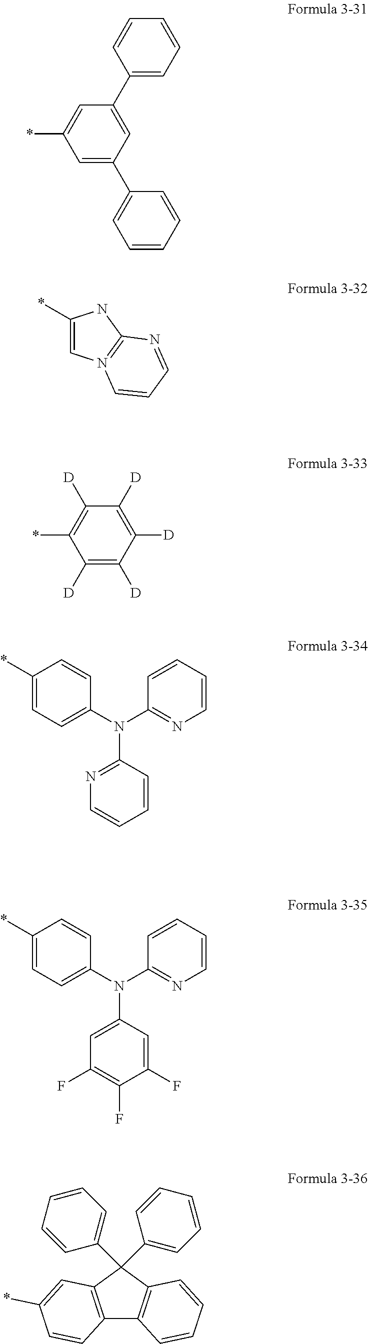 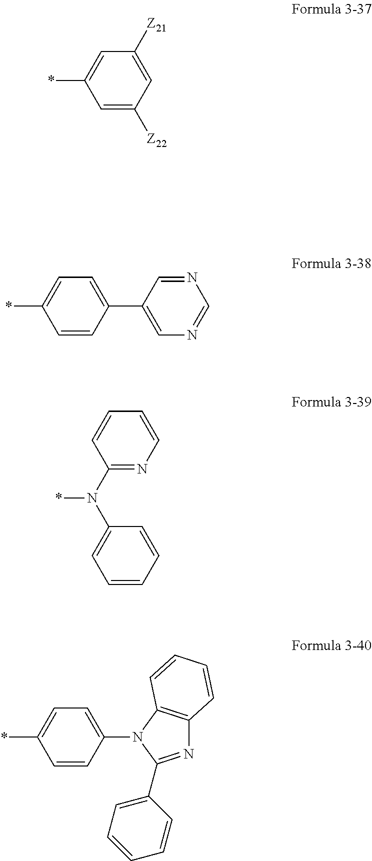
wherein Z₂₁ and Z₂₂ are each independently one selected from hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, a pentoxy group; and a methyl group, an ethyl group, a propyl group, a butyl group, a pentyl group, a methoxy group, an ethoxy group, a propoxy group, a butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof.

12. The condensed-cyclic compound of claim 1, wherein the condensed-cyclic compound is one of Compounds 1 through 76 below:

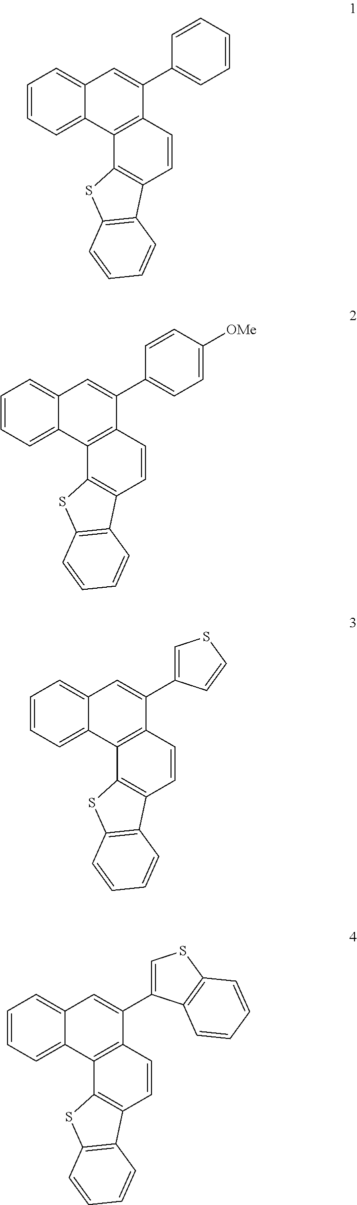 [SMILES: c1ccc(-c2cc3ccccc3c3c2ccc2c4ccccc4sc23)cc1 ; COc1ccc(-c2cc3ccccc3c3c2ccc2c4ccccc4sc23)cc1 ; c1ccc2c(c1)cc(-c1ccsc1)c1ccc3c4ccccc4sc3c12 ; c1ccc2c(c1)cc(-c1csc3ccccc13)c1ccc3c4ccccc4sc3c12]

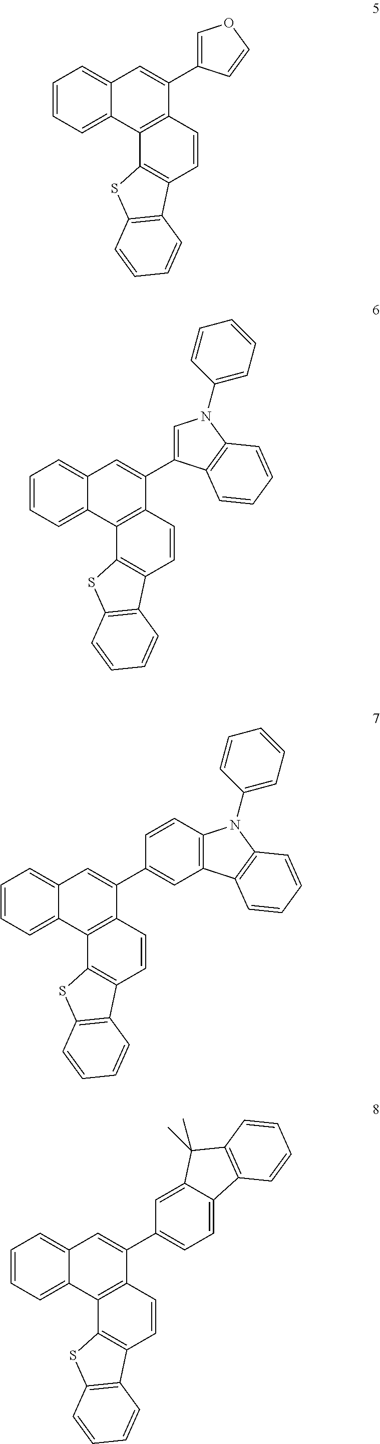 [SMILES: c1ccc2c(c1)cc(-c1ccoc1)c1ccc3c4ccccc4sc3c12 ; c1ccc(-n2cc(-c3cc4ccccc4c4c3ccc3c5ccccc5sc34)c3ccccc32)cc1 ; c1ccc(-n2c3ccccc3c3cc(-c4cc5ccccc5c5c4ccc4c6ccccc6sc45)ccc32)cc1 ; CC1(C)c2ccccc2-c2ccc(-c3cc4ccccc4c4c3ccc3c5ccccc5sc34)cc21]

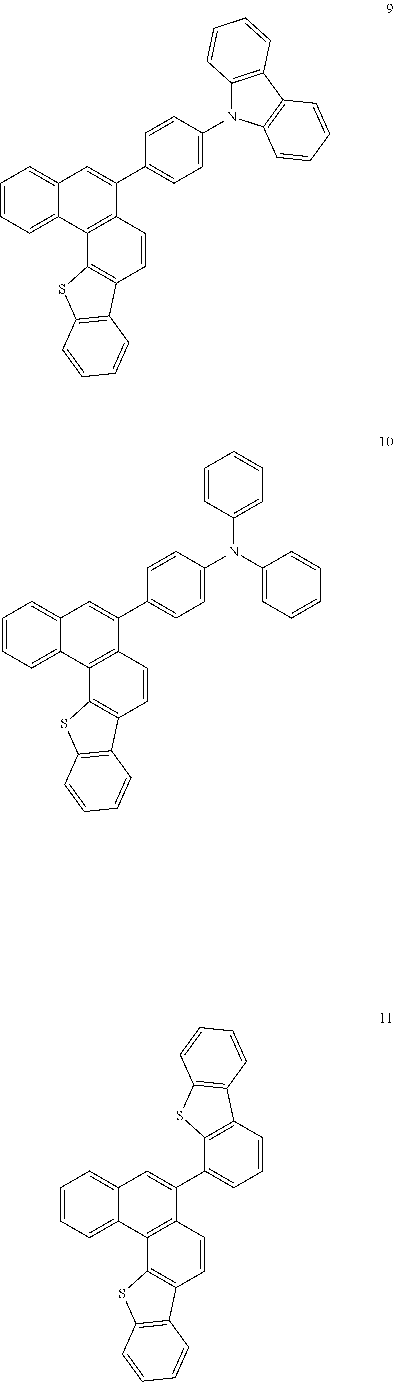 [SMILES: c1ccc2c(c1)cc(-c1ccc(-n3c4ccccc4c4ccccc43)cc1)c1ccc3c4ccccc4sc3c12 ; c1ccc(N(c2ccccc2)c2ccc(-c3cc4ccccc4c4c3ccc3c5ccccc5sc34)cc2)cc1 ; c1ccc2c(c1)cc(-c1cccc3c1sc1ccccc13)c1ccc3c4ccccc4sc3c12]

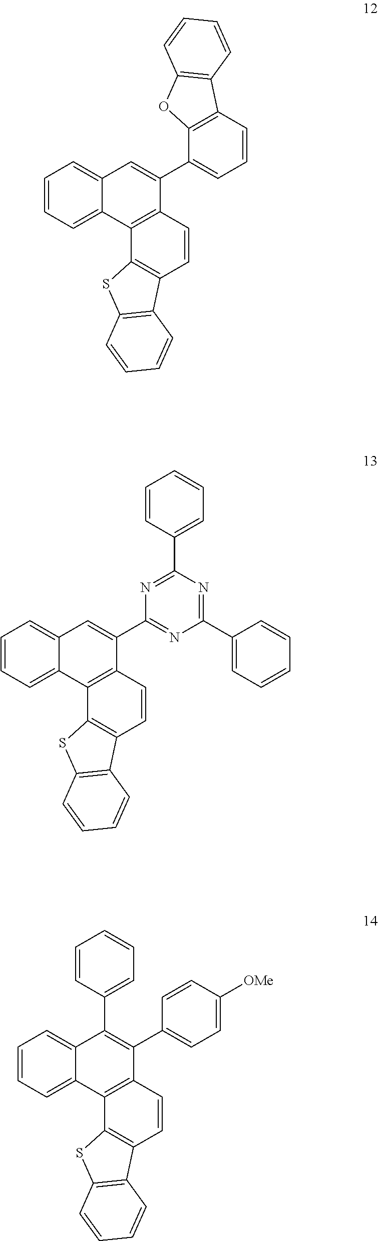 [SMILES: c1ccc2c(c1)cc(-c1cccc3c1oc1ccccc13)c1ccc3c4ccccc4sc3c12 ; c1ccc(-c2nc(-c3ccccc3)nc(-c3cc4ccccc4c4c3ccc3c5ccccc5sc34)n2)cc1 ; COc1ccc(-c2c(-c3ccccc3)c3ccccc3c3c2ccc2c4ccccc4sc23)cc1]

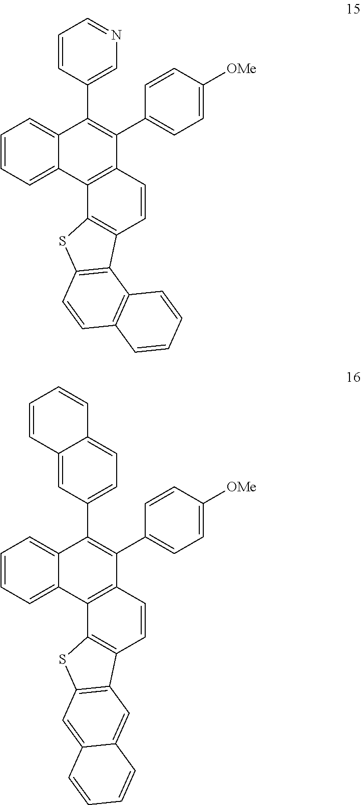 [SMILES: COc1ccc(-c2c(-c3cccnc3)c3ccccc3c3c2ccc2c3sc3ccc4ccccc4c32)cc1 ; COc1ccc(-c2c(-c3ccc4ccccc4c3)c3ccccc3c3c2ccc2c4cc5ccccc5cc4sc23)cc1]

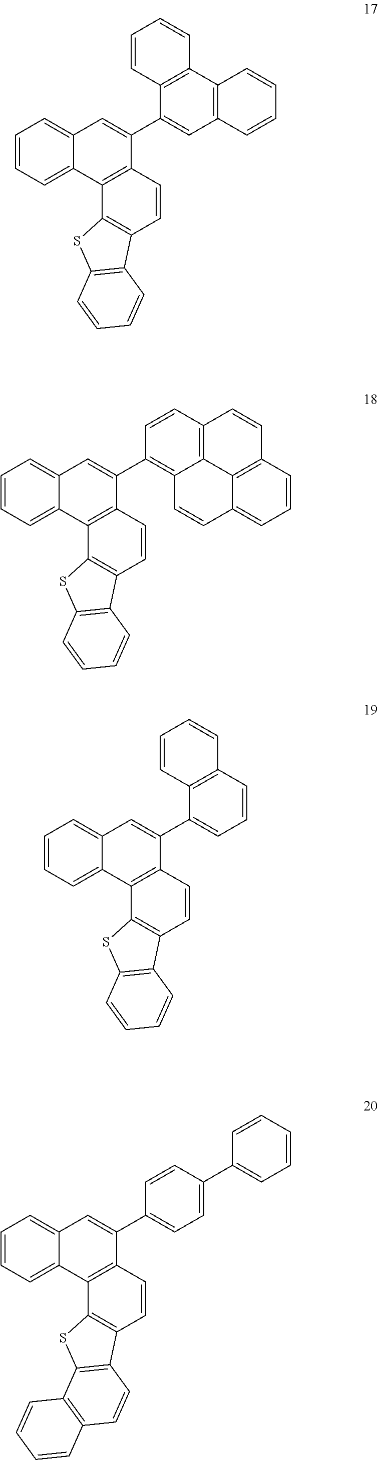 [SMILES: c1ccc2c(c1)cc(-c1cc3ccccc3c3c1ccc1c4ccccc4sc13)c1ccccc12 ; c1ccc2c(c1)cc(-c1ccc3ccc4cccc5ccc1c3c45)c1ccc3c4ccccc4sc3c12 ; c1ccc2c(-c3cc4ccccc4c4c3ccc3c5ccccc5sc34)cccc2c1 ; c1ccc(-c2ccc(-c3cc4ccccc4c4c3ccc3c5ccc6ccccc6c5sc34)cc2)cc1]

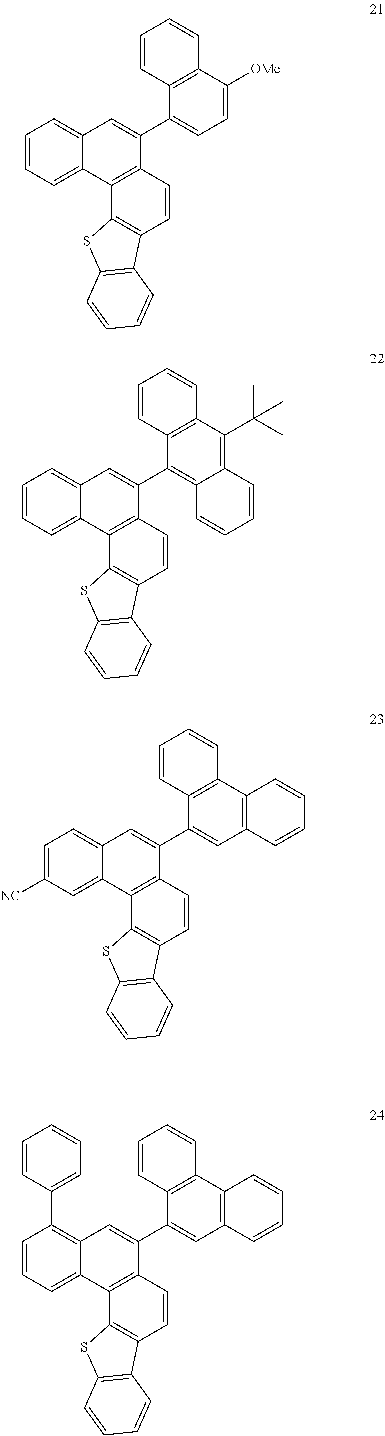 [SMILES: COc1ccc(-c2cc3ccccc3c3c2ccc2c4ccccc4sc23)c2ccccc12 ; CC(C)(C)c1c2ccccc2c(-c2cc3ccccc3c3c2ccc2c4ccccc4sc23)c2ccccc12 ; N#Cc1ccc2cc(-c3cc4ccccc4c4ccccc34)c3ccc4c5ccccc5sc4c3c2c1 ; c1ccc(-c2cccc3c2cc(-c2cc4ccccc4c4ccccc24)c2ccc4c5ccccc5sc4c23)cc1]

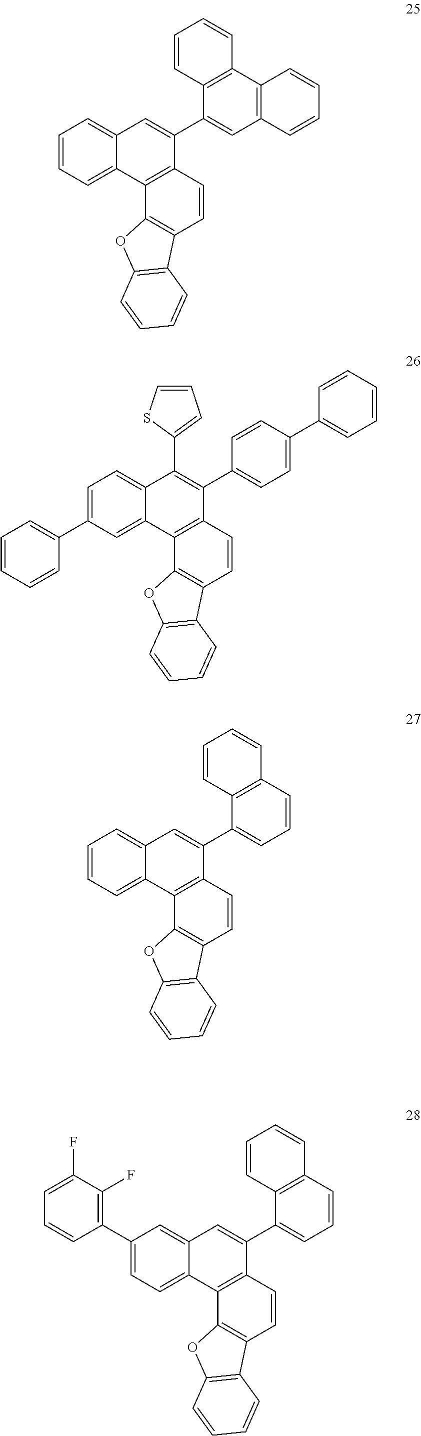 [SMILES: c1ccc2c(c1)cc(-c1cc3ccccc3c3c1ccc1c4ccccc4oc13)c1ccccc12 ; c1ccc(-c2ccc(-c3c(-c4cccs4)c4ccc(-c5ccccc5)cc4c4c3ccc3c5ccccc5oc34)cc2)cc1 ; c1ccc2c(-c3cc4ccccc4c4c3ccc3c5ccccc5oc34)cccc2c1 ; Fc1cccc(-c2ccc3c(c2)cc(-c2cccc4ccccc24)c2ccc4c5ccccc5oc4c23)c1F]

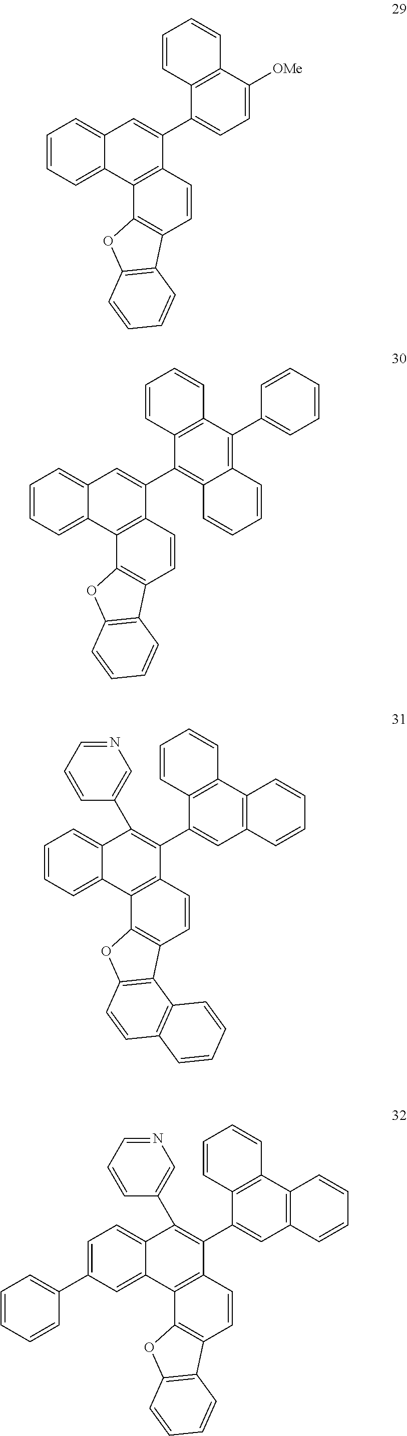

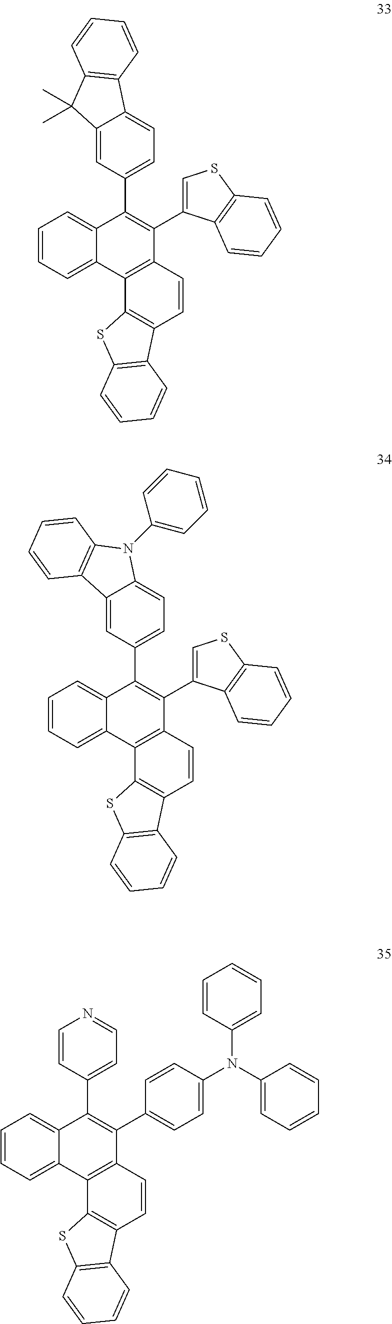 [SMILES: CC1(C)c2ccccc2-c2ccc(-c3c(-c4csc5ccccc45)c4ccc5c6ccccc6sc5c4c4ccccc34)cc21 ; c1ccc(-n2c3ccccc3c3cc(-c4c(-c5csc6ccccc56)c5ccc6c7ccccc7sc6c5c5ccccc45)ccc32)cc1 ; c1ccc(N(c2ccccc2)c2ccc(-c3c(-c4ccncc4)c4ccccc4c4c3ccc3c5ccccc5sc34)cc2)cc1]

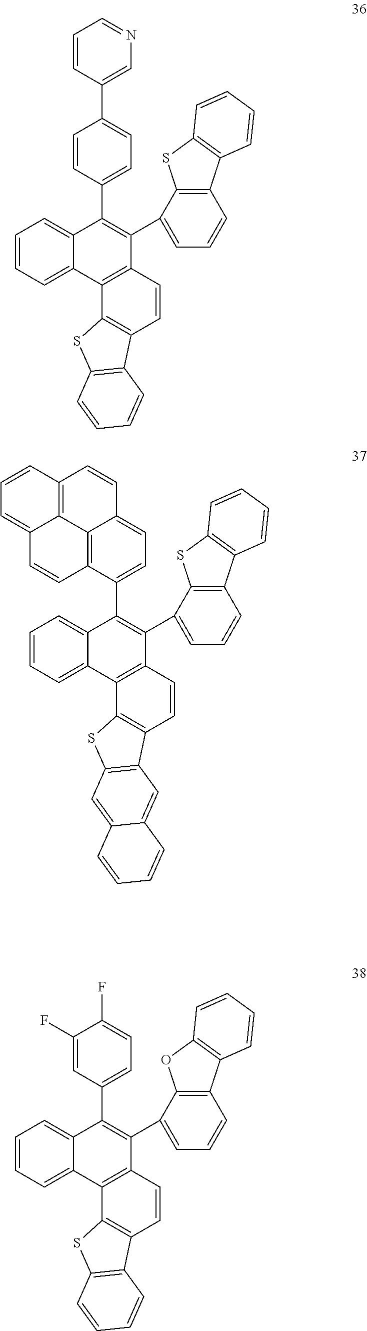

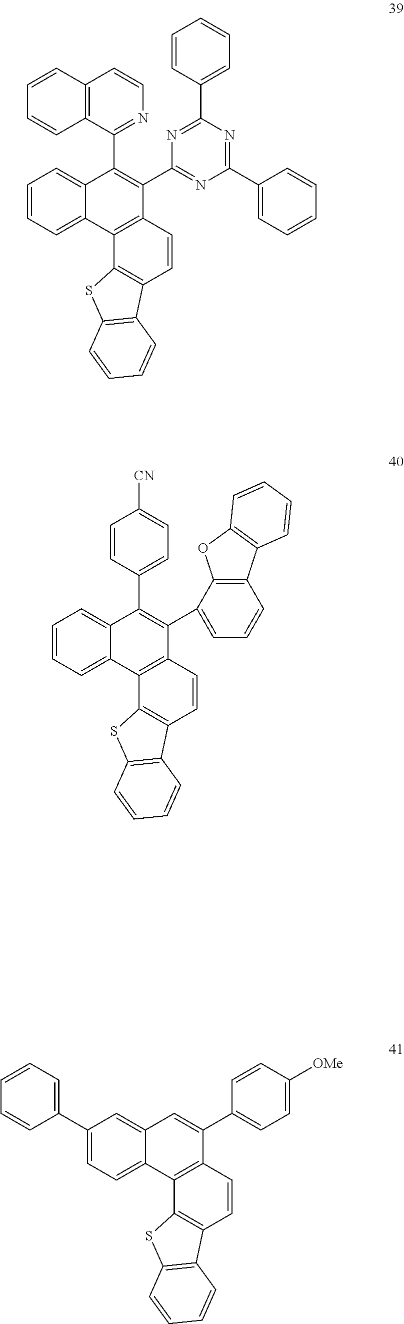 [SMILES: c1ccc(-c2nc(-c3ccccc3)nc(-c3c(-c4nccc5ccccc45)c4ccccc4c4c3ccc3c5ccccc5sc34)n2)cc1 ; N#Cc1ccc(-c2c(-c3cccc4c3oc3ccccc34)c3ccc4c5ccccc5sc4c3c3ccccc23)cc1 ; COc1ccc(-c2cc3cc(-c4ccccc4)ccc3c3c2ccc2c4ccccc4sc23)cc1]

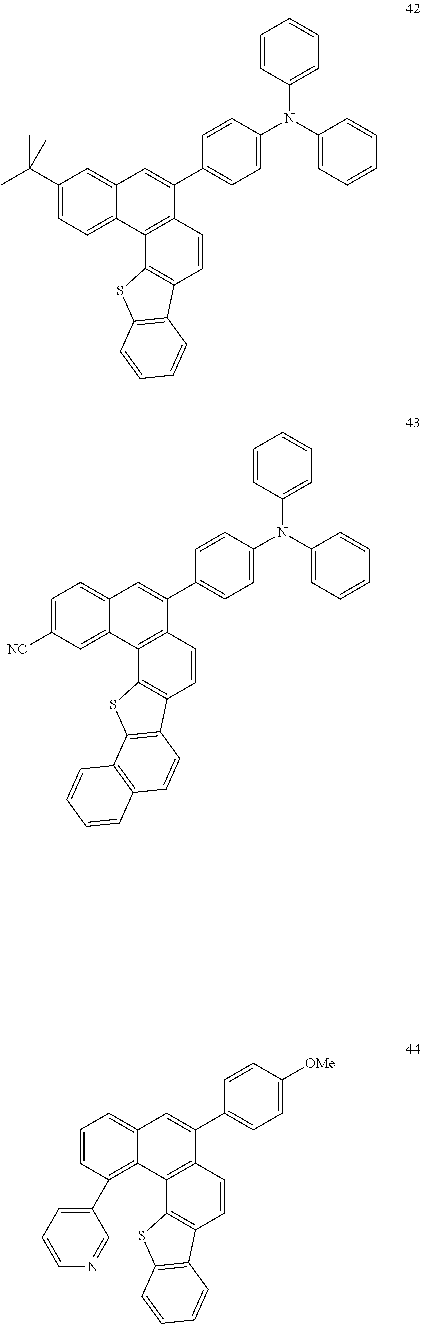 [SMILES: CC(C)(C)c1ccc2c(c1)cc(-c1ccc(N(c3ccccc3)c3ccccc3)cc1)c1ccc3c4ccccc4sc3c12 ; N#Cc1ccc2cc(-c3ccc(N(c4ccccc4)c4ccccc4)cc3)c3ccc4c5ccc6ccccc6c5sc4c3c2c1 ; COc1ccc(-c2cc3cccc(-c4cccnc4)c3c3c2ccc2c4ccccc4sc23)cc1]

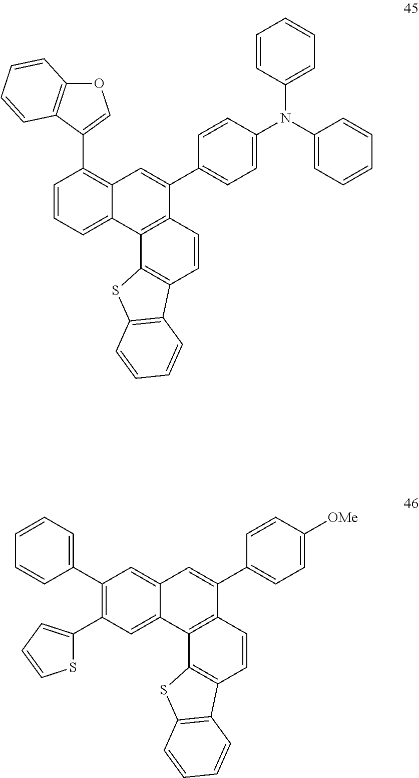 [SMILES: c1ccc(N(c2ccccc2)c2ccc(-c3cc4c(-c5coc6ccccc56)cccc4c4c3ccc3c5ccccc5sc34)cc2)cc1 ; COc1ccc(-c2cc3cc(-c4ccccc4)c(-c4cccs4)cc3c3c2ccc2c4ccccc4sc23)cc1]

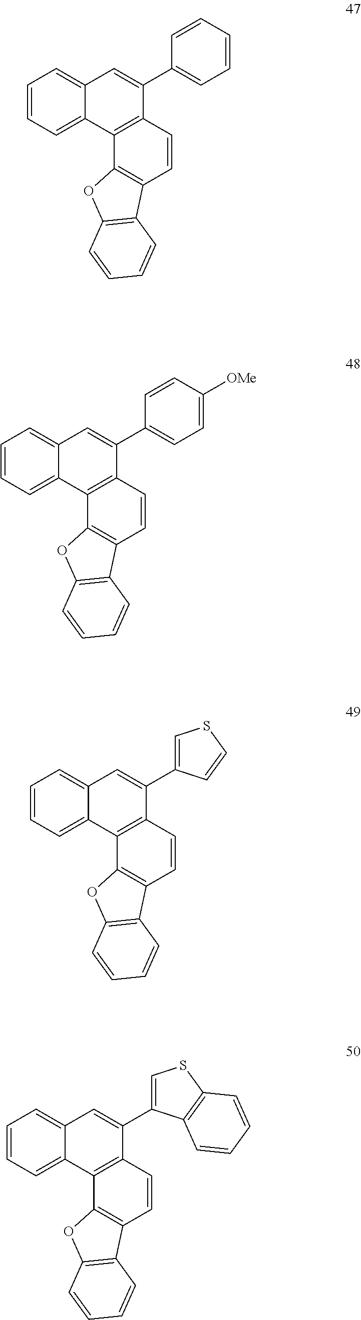 [SMILES: c1ccc(-c2cc3ccccc3c3c2ccc2c4ccccc4oc23)cc1 ; COc1ccc(-c2cc3ccccc3c3c2ccc2c4ccccc4oc23)cc1 ; c1ccc2c(c1)cc(-c1ccsc1)c1ccc3c4ccccc4oc3c12 ; c1ccc2c(c1)cc(-c1csc3ccccc13)c1ccc3c4ccccc4oc3c12]

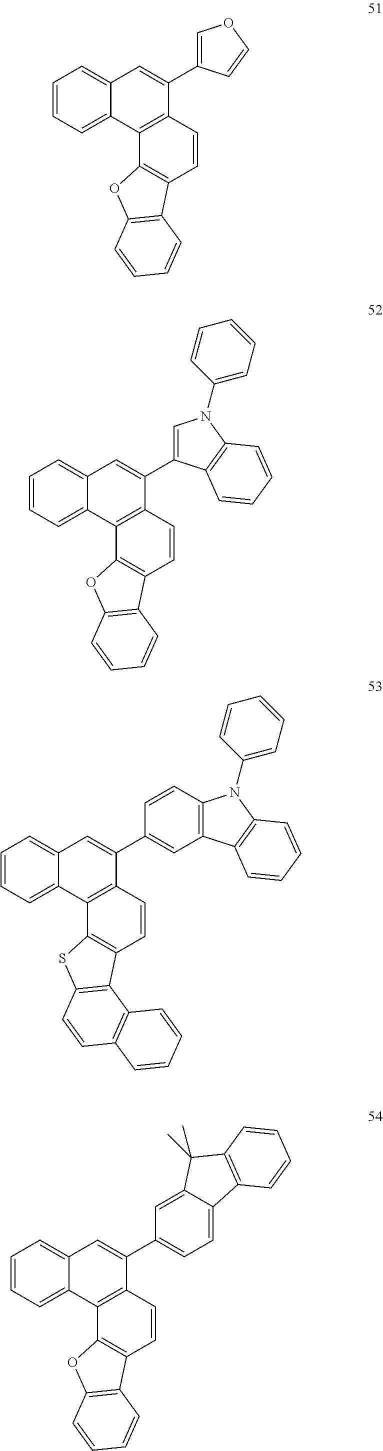 [SMILES: c1ccc2c(c1)cc(-c1ccoc1)c1ccc3c4ccccc4oc3c12 ; c1ccc(-n2cc(-c3cc4ccccc4c4c3ccc3c5ccccc5oc34)c3ccccc32)cc1 ; c1ccc(-n2c3ccccc3c3cc(-c4cc5ccccc5c5c4ccc4c5sc5ccc6ccccc6c54)ccc32)cc1 ; CC1(C)c2ccccc2-c2ccc(-c3cc4ccccc4c4c3ccc3c5ccccc5oc34)cc21]

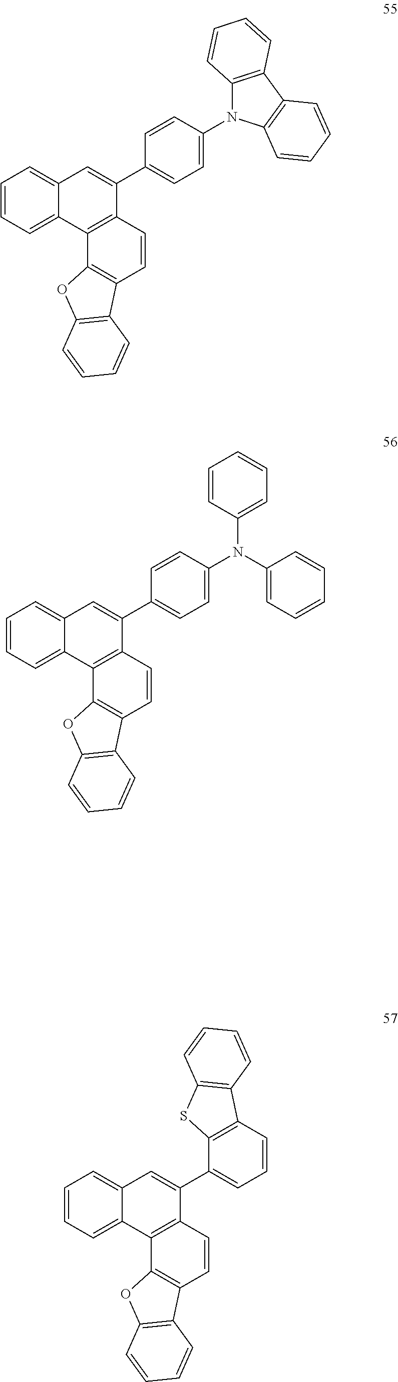 [SMILES: c1ccc2c(c1)cc(-c1ccc(-n3c4ccccc4c4ccccc43)cc1)c1ccc3c4ccccc4oc3c12 ; c1ccc(N(c2ccccc2)c2ccc(-c3cc4ccccc4c4c3ccc3c5ccccc5oc34)cc2)cc1 ; c1ccc2c(c1)cc(-c1cccc3c1sc1ccccc13)c1ccc3c4ccccc4oc3c12]

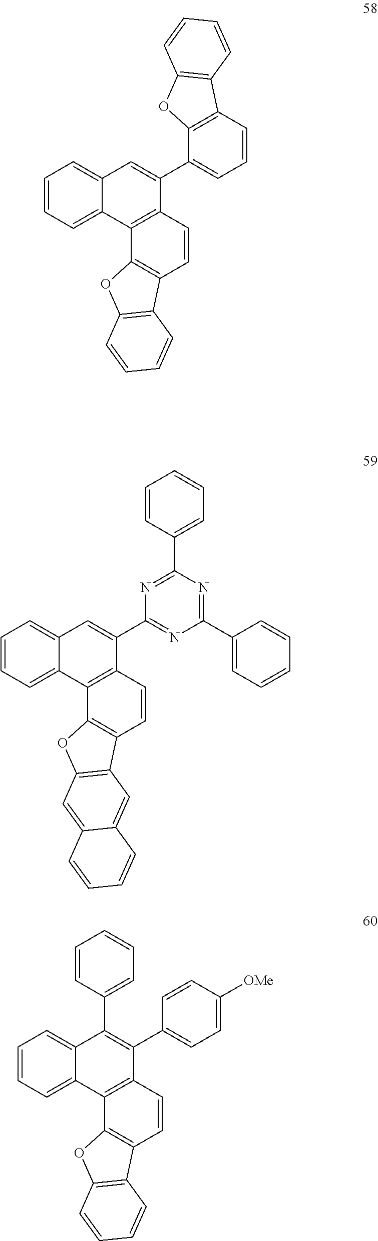

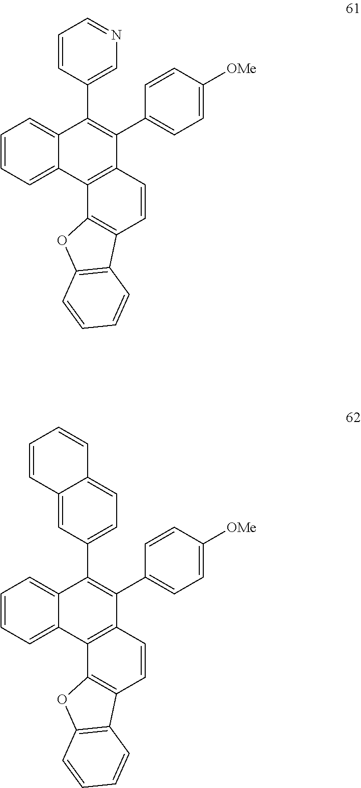 [SMILES: COc1ccc(-c2c(-c3cccnc3)c3ccccc3c3c2ccc2c4ccccc4oc23)cc1 ; COc1ccc(-c2c(-c3ccc4ccccc4c3)c3ccccc3c3c2ccc2c4ccccc4oc23)cc1]

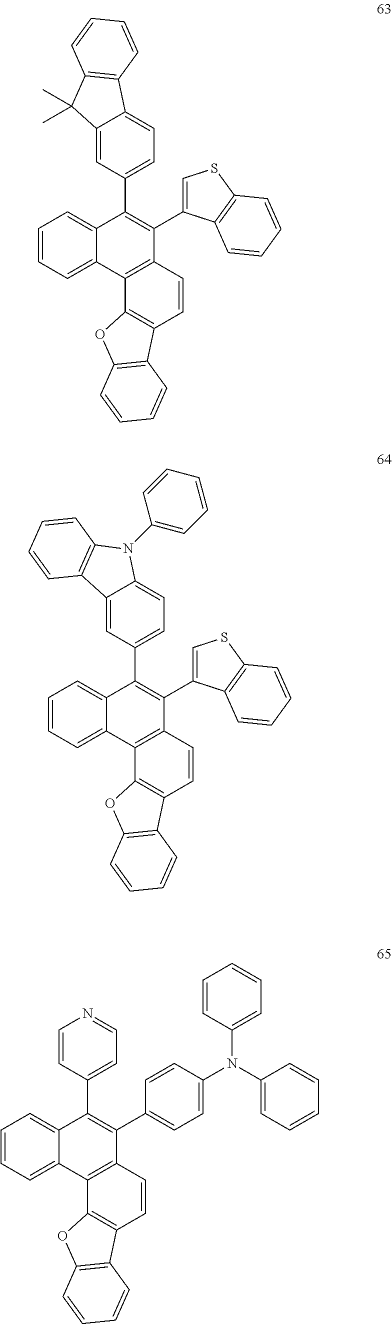 [SMILES: CC1(C)c2ccccc2-c2ccc(-c3c(-c4csc5ccccc45)c4ccc5c6ccccc6oc5c4c4ccccc34)cc21 ; c1ccc(-n2c3ccccc3c3cc(-c4c(-c5csc6ccccc56)c5ccc6c7ccccc7oc6c5c5ccccc45)ccc32)cc1 ; c1ccc(N(c2ccccc2)c2ccc(-c3c(-c4ccncc4)c4ccccc4c4c3ccc3c5ccccc5oc34)cc2)cc1]

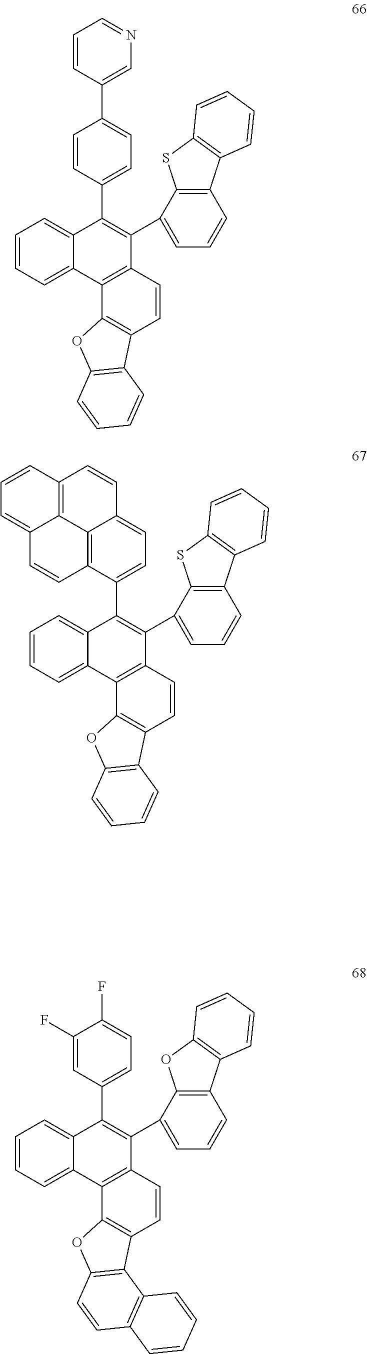

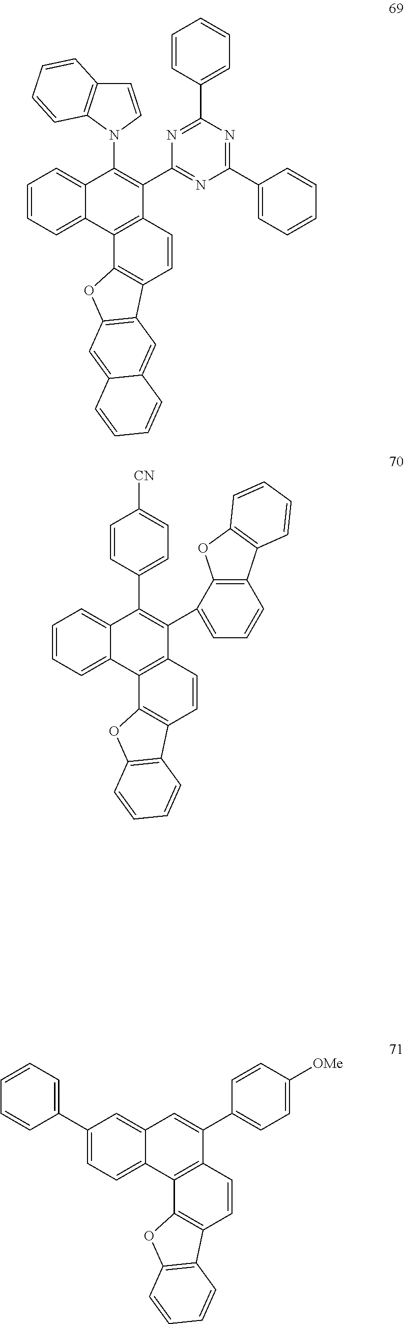

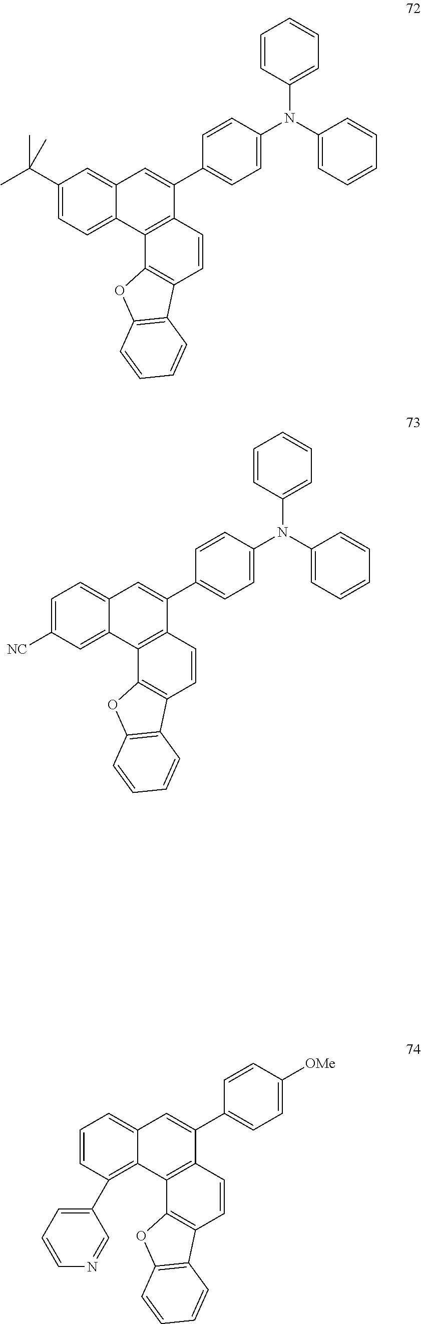 [SMILES: CC(C)(C)c1ccc2c(c1)cc(-c1ccc(N(c3ccccc3)c3ccccc3)cc1)c1ccc3c4ccccc4oc3c12 ; N#Cc1ccc2cc(-c3ccc(N(c4ccccc4)c4ccccc4)cc3)c3ccc4c5ccccc5oc4c3c2c1 ; COc1ccc(-c2cc3cccc(-c4cccnc4)c3c3c2ccc2c4ccccc4oc23)cc1]

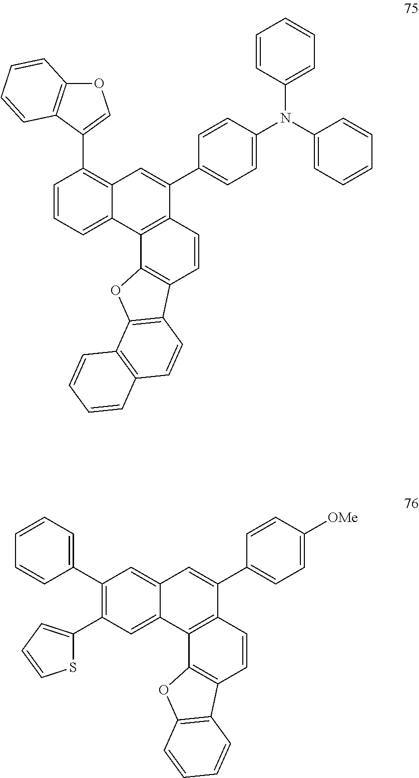 [SMILES: c1ccc(N(c2ccccc2)c2ccc(-c3cc4c(-c5coc6ccccc56)cccc4c4c3ccc3c5ccc6ccccc6c5oc34)cc2)cc1 ; COc1ccc(-c2cc3cc(-c4ccccc4)c(-c4cccs4)cc3c3c2ccc2c4ccccc4oc23)cc1]

13. A method of preparing a condensed-cyclic compound represented by Formula 1 below, the method performed according to Reaction Scheme 1 below and comprising:
reacting Intermediate c-(1) below with 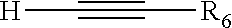
to obtain Intermediate c;
reacting the Intermediate c with Intermediate d′ or d″ to obtain Intermediate e; and
cyclizing the Intermediate e to obtain the condensed-cyclic compound of Formula 1: 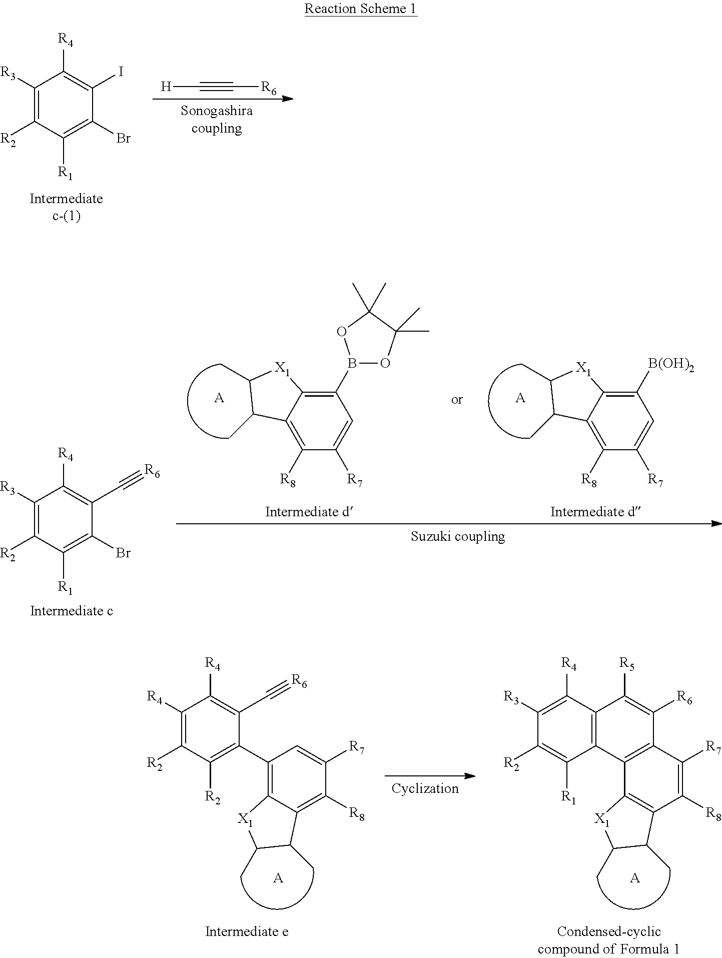 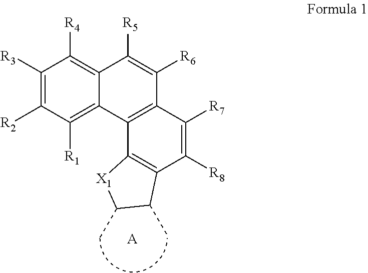
wherein X₁ is N(R₁₀), S, or O;
a ring A is a substituted or unsubstituted aromatic ring;
R₁ through R₈ and R₁₀ are each independently hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, a substituted or unsubstituted C₂-C₆₀ heteroaryl group, —Si(R₂₁)(R₂₂)(R₂₃), or N(R₂₄)(R₂₅); and
R₂₁ through R₂₅ are each independently a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ aryl group, a substituted or unsubstituted C₅-C₆₀ aryloxy group, a substituted or unsubstituted C₅-C₆₀ arylthio group, or a substituted or unsubstituted C₂-C₆₀ heteroaryl group.

14. The method of claim 13, wherein the condensed-cyclic compound is represented by one of Formulae 1A-1 through 1D-3 below: 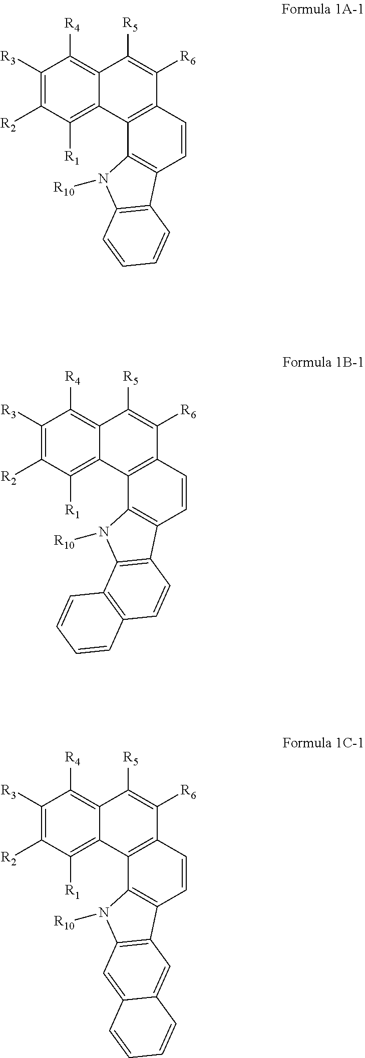 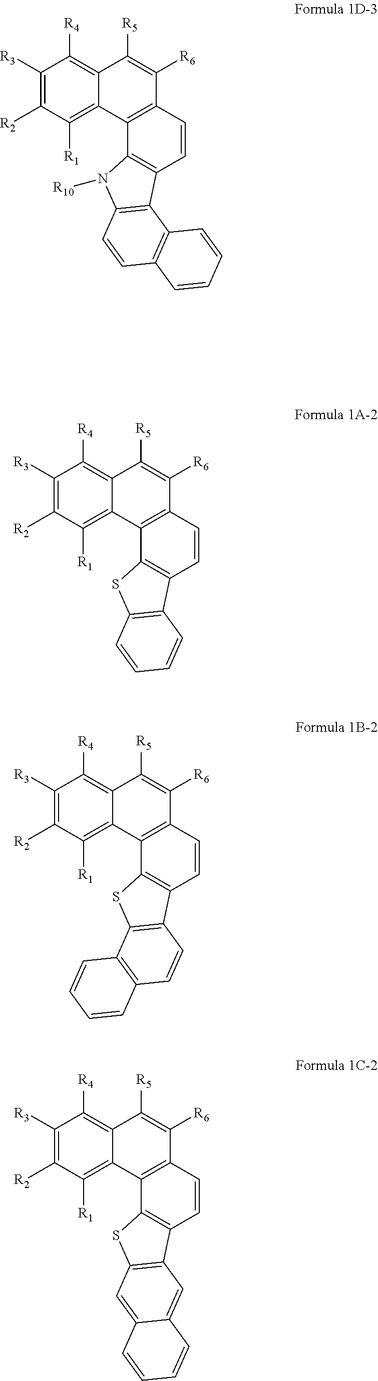 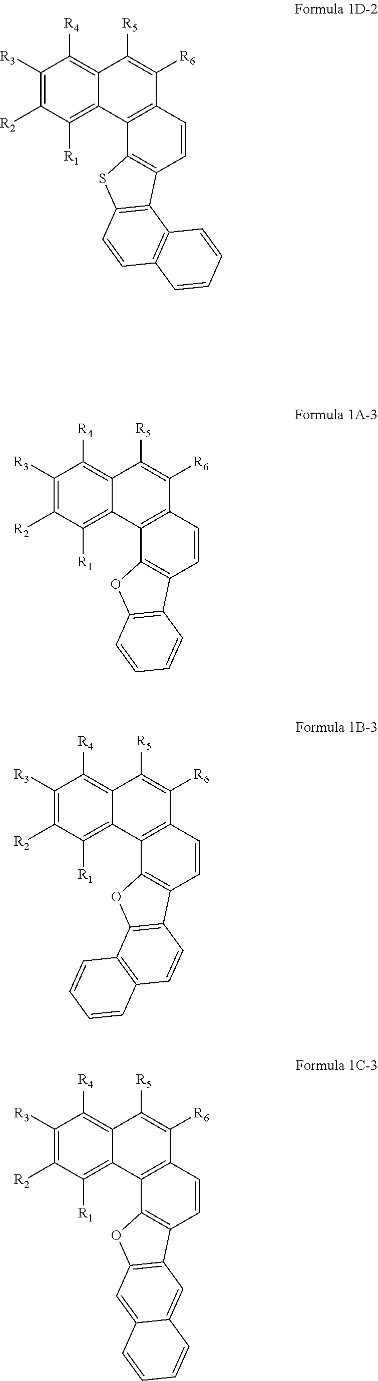 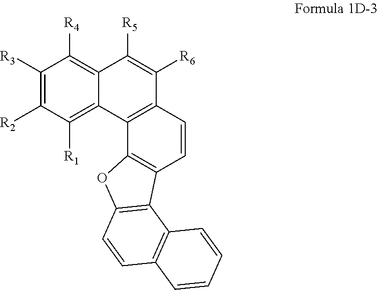
wherein R₁ through R₆ and R₁₀ are each independently one of hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a C₁-C₁₀ alkyl group, a C₁-C₁₀ alkoxy group, and Formulae 3-1 through 3-40 below: 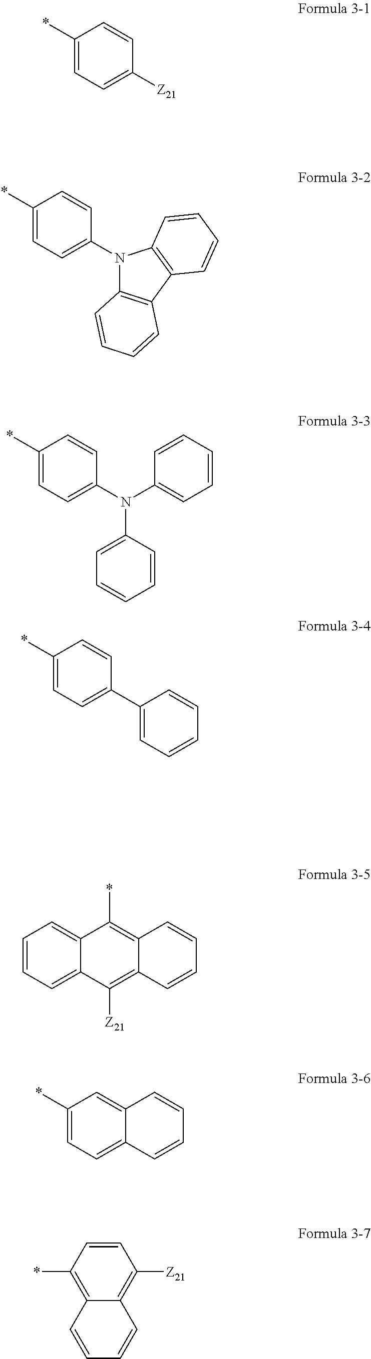 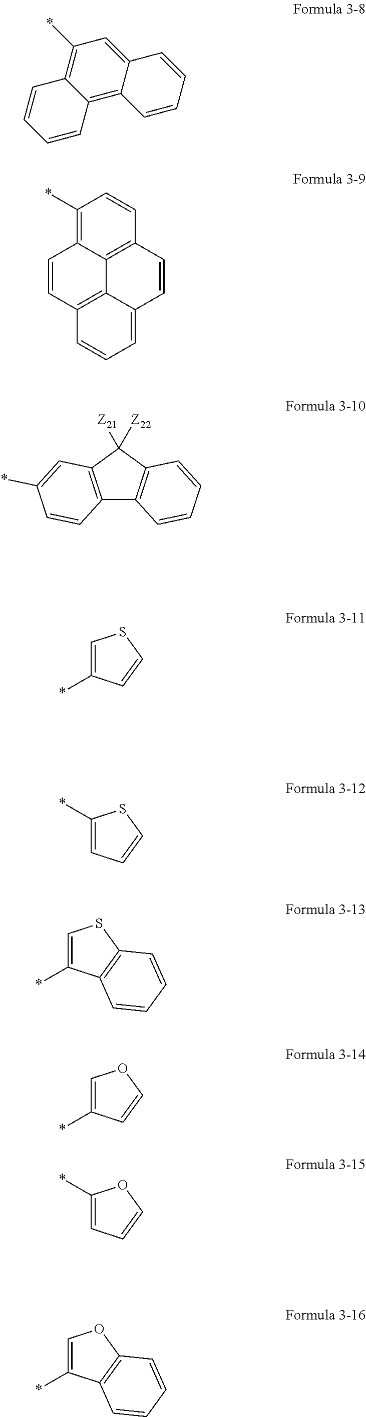 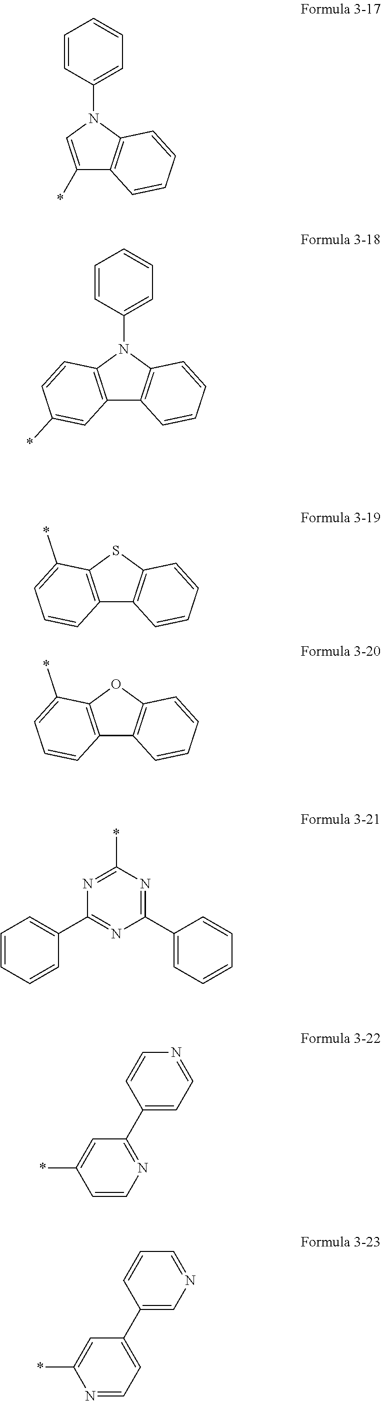 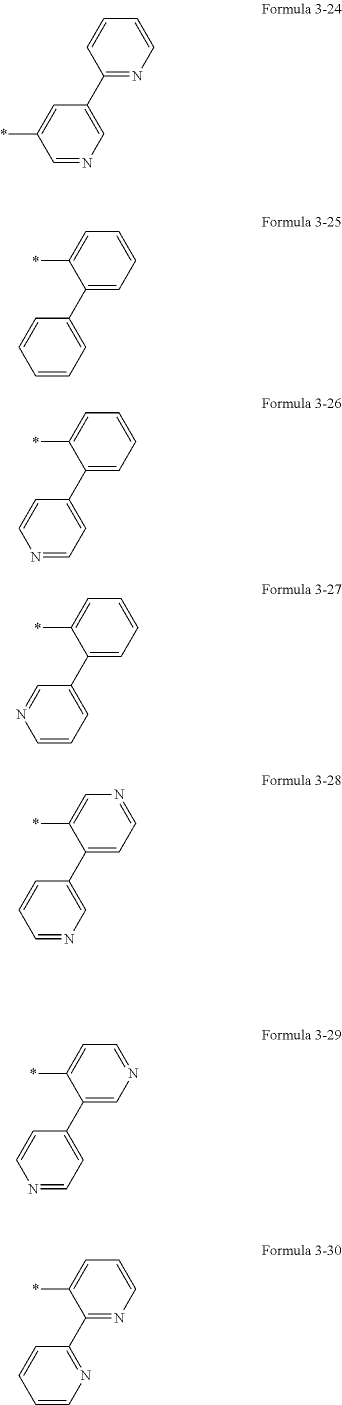 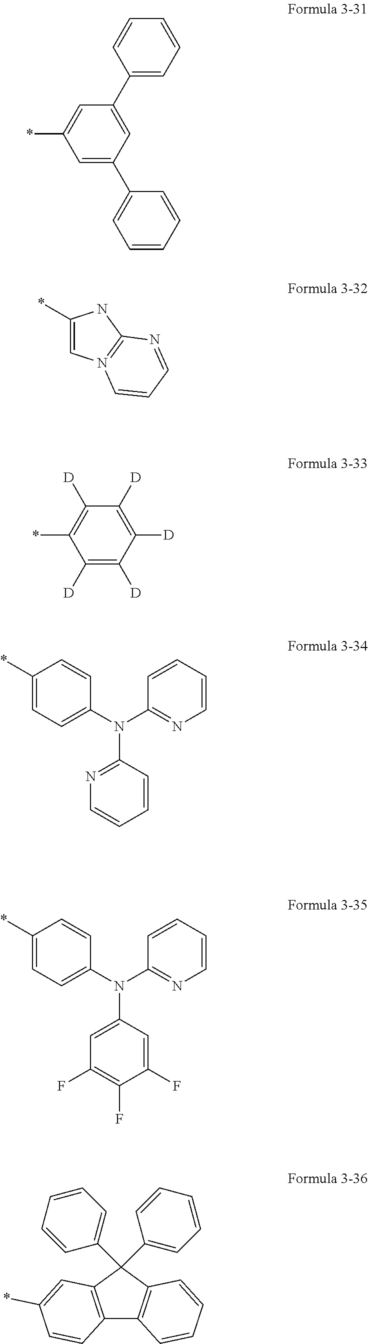 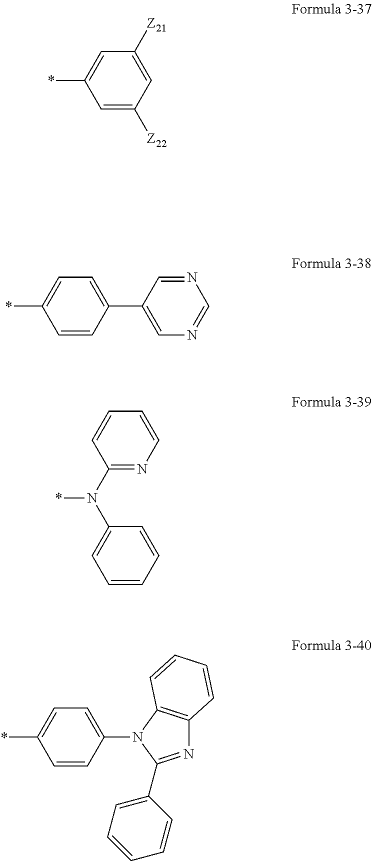
wherein Z₂₁ and Z₂₂ are each independently one selected from hydrogen, deuterium, a halogen atom, a hydroxyl group, a cyano group, a nitro group, an amino group, an amidino group, hydrazine, hydrazone, a carboxyl group or a salt thereof, a sulfonic acid group or a salt thereof, a phosphoric acid or a salt thereof, a methyl group, an ethyl group, a propyl group, a butyl group, a pentyl group, a methoxy group, an ethoxy group, a propoxy group, a butoxy group, a pentoxy group; and a methyl group, an ethyl group, a propyl group, a butyl group, a pentyl group, a methoxy group, an ethoxy group, a propoxy group, a butoxy group, and a pentoxy group that is substituted with at least one of deuterium, a halogen atom, a hydroxyl group, a cyano group, a nitro group, an amino group, an amidino group, hydrazine, hydrazone, a carboxyl group or a salt thereof, a sulfonic acid group or a salt thereof, and a phosphoric acid or a salt thereof.

15. An organic light-emitting device comprising
a first electrode;
a second electrode facing the first electrode; and
a first layer interposed between the first electrode and the second electrode, wherein the first layer comprises at least one of the condensed-cyclic compounds according to claim 1.

16. The organic light-emitting device of claim 15, wherein the first layer comprises at least one of a hole injection layer, a hole transport layer, a functional layer having hole injection and hole, transport abilities, an emission layer, an electron transport layer, and an electron injection layer.

17. The organic light-emitting device of claim 16, wherein the first layer comprises an emission layer and the emission layer comprises the condensed-cyclic compound.

18. The organic light-emitting device of claim 17, wherein the emission layer further comprises a phosphorescent dopant and the condensed-cyclic compound in the emission layer acts as a phosphorescent host.

19. The organic light-emitting device of claim 16, wherein the first layer comprises an emission layer and an electron transport layer, wherein each of the emission layer and the electron transport layer comprises the condensed-cyclic compound, wherein the condensed-cyclic compound in the emission layer is different from the condensed-cyclic compound in the electron transport layer.

20. The organic light-emitting device of claim 19, wherein the emission layer further comprises a phosphorescent dopant and the condensed-cyclic compound in the emission layer acts as a phosphorescent host.
